SEQUENCE LISTING (36 sequences)

SEQ ID NO: 1 - DNA, 3861 bp, Artificial Sequence
  CU-CH2
atgacccagttcgaaggtttcaccaacctgtaccaggtttctaaaaccctgcgtttcgaactgatcccgcagggtaaaaccctgaaacacatccaggaacagggtttcatcgaagaagacaaagcgcgtaacgaccactacaaagaactgaaaccgatcatcgaccgtatctacaaaacctacgcggaccagtgcctgcagctggttcagctggactgggaaaacctgtctgcggcgatcgactcttaccgtaaagaaaaaaccgaagaaacccgtaacgcgctgatcgaagaacaggcgacctaccgtaacgcgatccacgactacttcatcggtcgtaccgacaacctgaccgacgcgatcaacaaacgtcacgcggaaatctacaaaggtctgttcaaagcggaactgttcaacggtaaagttctgaaacagctgggtaccgttaccaccaccgaacacgaaaacgcgctgctgcgttctttcgacaaattcaccacctacttctctggtttctacgaaaaccgtaaaaacgttttctctgcggaagacatctctaccgcgatcccgcaccgtatcgttcaggacaacttcccgaaattcaaagaaaactgccacatcttcacccgtctgatcaccgcggttccgtctctgcgtgaacacttcgaaaacgttaaaaaagcgatcggtatcttcgtttctacctctatcgaagaagttttctctttcccgttctacaaccagctgctgacccagacccagatcgacctgtacaaccagctgctgggtggtatctctcgtgaagcgggtaccgaaaaaatcaaaggtctgaacgaagttctgaacctggcgatccagaaaaacgacgaaaccgcgcacatcatcgcgtctctgccgcaccgtttcatcccgcttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 2 - DNA, 3876 bp, Artificial Sequence
  CU-CH1
atggatagtttgaaagatttcaccaatctgtaccctgtcagtaagacattgagatttgaattaaagcccgttggaaagactttagaaaatatcgagaaagcaggtattttgaaagaggatgagcatcgtgcagaaagttatcggagggtgaagaaaataattgatacttatcataaggtatttatcgattcttctcttgaaaatatggctaaaatgggtattgagaatgaaataaaagcaatgctccaaagtttctgcgaattgtataaaaaagatcatcgcactgagggtgaagacaaggcattagataaaattcgagcagtacttcgtggcctgattgttggggctttcactggtgtttgcggaagacgggaaaatacagtccaaaacgagaagtacgagagtttgttcaaagaaaagttgataaaagaaattttacctgattttgtgctctctactgaggctgaaagcttgcctttctctgttgaagaagctacgaggtcactgaaggagtttgatagctttacatcctactttgctggtttttacgagaatagaaagaatatatactcgacgaaacctcaatccactgccattgcttatcgtcttattcatgagaacttgccgaagttcattgataatattcttgtttttcagaagatcaaagagcctatagccaaagagctggaacatattcgtgcggacttttctgccggggggtacataaaaaaggatgagagattggaggatattttttcgttgaactattatatccacgtgttatctcaggctgggatcgaaaaatataacgcattgattgggaagattgtgacagaaggagatggagagatgaaagggctcaatgaacacatcaacctttacaaccaacaaagaggcagagaggatcggctccctctttttaggcctcttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 3 - DNA, 3849 bp, Artificial Sequence
  CU-CH5 (M21)
atgactaaaacatttgattcagagttttttaatttgtactcgctgcaaaaaacggtacgctttgagttaaaacccgtgggagaaaccgcgtcatttgtggaagactttaaaaacgagggcttgaaacgtgttgtgagcgaagatgaaaggcgagccgtcgattaccagaaagttaaggaaataattgacgattaccatcgggatttcattgaagaaagtttaaattattttccggaacaggtgagtaaagatgctcttgagcaggcgtttcatctttatcagaaactgaaggcagcaaaagttgaggaaagggaaaaagcgctgaaagaatgggaagcgctgcagaaaaagctacgtgaaaaagtggtgaaatgcttctcggactcgaataaagcccgcttctcaaggattgataaaaaggaactgattaaggaagacctgataaattggttggtcgcccagaatcgcgaggatgatatccctacggtcgaaacgtttaacaacttcaccacatattttaccggcttccatgagaatcgtaaaaatatttactccaaagatgatcacgccaccgctattagctttcgccttattcatgaaaatcttccaaagttttttgacaacgtgattagcttcaataagttgaaagagggtttccctgaattaaaatttgataaagtgaaagaggatttagaagtagattatgatctgaagcatgcgtttgaaatagaatatttcgttaacttcgtgacccaagcgggcatagatcagtataattatctgttaggagggaaaaccctggaggacgggacgaaaaaacaagggatgaatgagcaaattaatctgttcaaacaacagcaaacgcgagataaagcgcgtcagattcccaaactgatcccccttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 4 - DNA, 3819 bp, Artificial Sequence
  CU-CH4
atgactaaaacatttgattcagagttttttaatttgtactcgctgcaaaaaacggtacgctttgagttaaaacccgtgggagaaaccgcgtcatttgtggaagactttaaaaacgagggcttgaaacgtgttgtgagcgaagatgaaaggcgagccgtcgattaccagaaagttaaggaaataattgacgattaccatcgggatttcattgaagaaagtttaaattattttccggaacaggtgagtaaagatgctcttgagcaggcgtttcatctttatcagaaactgaaggcagcaaaagttgaggaaagggaaaaagcgctgaaagaatgggaagcgctgcagaaaaagctacgtgaaaaagtggtgaaatgcttctcggactcgaataaagcccgcttctcaaggattgataaaaaggaactgattaaggaagacctgataaattggttggtcgcccagaatcgcgaggatgatatccctacggtcgaaacgtttaacaactttgcgactagctttaaagattacttcaagaaccgtgcaaattgcttttcagcggacgatatttcatcaagcagctgccatcgcatcgtcaacgacaatgcagagatattcttttcaaatgcgctggtctaccgccggatcgtaaaatcgctgagcaatgacgatatcaacaaaatttcgggcgatatgaaagattcattaaaagaaatgagtctggaagaaatatattcttacgagaagtatggggaatttattacccaggaaggcattagcttctataatgatatctgtgggaaagtgaattcttttatgaacctgtattgtcagaaaaataaagaaaacaaaaatttatacaaacttcagaaacttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 5 - DNA, 3810 bp, Artificial Sequence
  CU-CH3
atgaccaataaattcactaaccagtattctctctctaagaccctgcgctttgaactgattccgcaggggaaaaccttggagttcattcaagaaaaaggcctcttgtctcaggataaacagagggctgaatcttaccaagaaatgaagaaaactattgataagtttcataaatatttcattgatttagccttgtctaacgccaaattaactcacttggaaacgtatctggagttatacaacaaatctgccgaaactaagaaagaacagaaatttaaagacgatttgaaaaaagtacaggacaatctgcgtaaagaaattgtcaaatccttcagtgacggcgatgctaaaagcatttttgccattctggacaaaaaagagttgattactgtggaattagaaaagtggtttgaaaacaatgagcagaaagacatctacttcgatgagaaattcaaaactttcaccacctattttacaggatttcatcaaaaccggaagaacatgtactcagtagaaccgaactccacggccattgcgtatcgtttgatccatgagaatctgcctaaatttctggagaatgcgaaagcctttgaaaagattaagcaggtcgaatcgctgcaagtgaattttcgtgaactcatgggcgaatttggtgacgaaggtctaatcttcgttaacgaactggaagaaatgtttcagattaattactacaatgacgtgctatcgcagaacggtatcacaatctacaatagtattatctcagggttcacaaaaaacgatataaaatacaaaggcctgaacgagtatatcaataactacaaccaaacaaaggacaaaaaggataggcttccgaaactgaagcagcttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 6 - DNA, 3861 bp, Artificial Sequence
  CU-CH6
atgactaaaacatttgattcagagttttttaatttgtactcgctgcaaaaaacggtacgctttgagttaaaacccgtgggagaaaccgcgtcatttgtggaagactttaaaaacgagggcttgaaacgtgttgtgagcgaagatgaaaggcgagccgtcgattaccagaaagttaaggaaataattgacgattaccatcgggatttcattgaagaaagtttaaattattttccggaacaggtgagtaaagatgctcttgagcaggcgtttcatctttatcagaaactgaaggcagcaaaagttgaggaaagggaaaaagcgctgaaagaatgggaagcgctgcagaaaaagctacgtgaaaaagtggtgaaatgcttctcggactcgaataaagcccgcttctcaaggattgataaaaaggaactgattaaggaagacctgataaattggttggtcgcccagaatcgcgaggatgatatccctacggtcgaaacgtttaacaacttcaccacatattttaccggcttccatgagaatcgtaaaaatatttactccaaagatgatcacgccaccgctattagctttcgccttattcatgaaaatcttccaaagttttttgacaacgtgattagcttcaataagttgaaagagggtttccctgaattaaaatttgataaagtgaaagaggatttagaagtagattatgatctgaagcatgcgtttgaaatagaatatttcgttaacttcgtgacccaagcgggcatagatcagtataattatctgttaggagggaaaaccctggaggacgggacgaaaaaacaagggatgaatgagcaaattaatctgttcaaacaacagcaaacgcgagataaagcgcgtcagattcccaaactgatcccccttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaagattgatccgaccacgggcttcgccaatgttctgaatctgtcgaaggtacgcaatgttgatgcgatcaaaagctttttttctaacttcaacgaaattagttatagcaagaaagaagcccttttcaaattctcattcgatctggattcactgagtaagaaaggctttagtagctttgtgaaatttagtaagagtaaatggaacgtctacacctttggagaacgtatcataaagccaaagaataagcaaggttatcgggaggacaaaagaatcaacttgaccttcgagatgaagaagttacttaacgagtataaggtttcttttgatcttgaaaataacttgattccgaatctcacgagtgccaacctgaaggatactttttggaaagagctattctttatcttcaagactacgctgcagctccgtaacagcgttactaacggtaaagaagatgtgctcatctctccggtcaaaaatgcgaagggtgaattcttcgtttcgggaacgcataacaagactcttccgcaagattgcgatgcgaacggtgcataccatattgcgttgaaaggtctgatgatactcgaacgtaacaaccttgtacgtgaggagaaagatacgaaaaagattatggcgatttcaaacgtggattggttcgagtacgtgcagaaacgtagaggcgttctgtaa

SEQ ID NO: 7 - DNA, 3789 bp, Artificial Sequence
  CU-CH7
atgaacaactacgacgaattcaccaaactgtacccgatccagaaaaccatccgtttcgaactgaaaccgcagggtcgtaccatggaacacctggaaaccttcaacttcttcgaagaagaccgtgaccgtgcggaaaaatacaaaatcctgaaagaagcgatcgacgaataccacaaaaaattcatcgacgaacacctgaccaacatgtctctggactggaactctctgaaacagatctctgaaaaatactacaaatctcgtgaagaaaaagacaaaaaagttttcctgtctgaacagaaacgtatgcgtcaggaaatcgtttctgaattcaaaaaagacgaccgtttcaaagacctgttctctaaaaaactgttctctgaactgctgaaagaagaaatctacaaaaaaggtaaccaccaggaaatcgacgcgctgaaatctttcgacaaattctctggttacttcatcggtctgcacgaaaaccgtaaaaacatgtactctgacggtgacgaaatcaccgcgatctctaaccgtatcgttaacgaaaacttcccgaaattcctggacaacctgcagaaataccaggaagcgcgtaaaaaatacccggaatggatcatcaaagcggaatctgcgctggttgcgcacaacatcaaaatggacgaagttttctctctggaatacttcaacaaagttctgaaccaggaaggtatccagcgttacaacctggcgctgggtggttacgttaccaaatctggtgaaaaaatgatgggtctgaacgacgcgctgaacctggcgcaccagtctgaaaaatcttctaaaggtcgtatccacatgaccccgcttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 8 - DNA, 3795 bp, Artificial Sequence
  CU-CH8
atgcatacaggcggtcttcttagtatggacgcgaaagagttcacaggtcagtatccgttgtcgaaaacattacgattcgaacttcggcccatcggccgcacgtgggataacctggaggcctcaggctacttagcggaagaccgccatcgtgccgaatgttatcctcgtgcgaaagagttattggatgacaaccatcgtgccttcctgaatcgtgtgttgccacaaatcgatatggattggcacccgattgcggaggccttttgtaaggtacataaaaaccctggtaataaagaacttgcccaggattacaaccttcagttgtcaaagcgccgtaaggagatcagcgcatatcttcaggatgcagatggctataaaggcctgttcgcgaagcccgccttagacgaagctatgaaaattgcgaaagaaaacgggaacgaaagtgatattgaggttctcgaagcgtttaacggttttagcgtatacttcaccggttatcatgagtcacgcgagaacatttatagcgatgaggatatggtgagcgtagcctaccgaattactgaggataatttcccgcgctttgtctcaaacgctttgatctttgataaattaaacgaaagccatccggatattatctctgaagtatcgggcaatcttggagttgatgacattggtaagtactttgacgtgtcgaactataacaattttctttcccaggccggtatagatgactacaatcacattattggcggccatacaaccgaagacggactgatacaagcgtttaatgtcgtattgaacttacgtcaccaaaaagaccctggctttgaaaaaattcagttcaaacagcttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 9 - DNA, 3849 bp, Artificial Sequence; identical to SEQ ID NO: 3
  CU-CH9 (M44)

SEQ ID NO: 10 - DNA, 1033 bp, Artificial Sequence
  SD_Cpf1_1
aagcattggccgtaagtgcgattccggaaaggagatatacatgtcatcgctcacgaaattcactaacaaatactctaaacagctcaccattaagaatgaactcatcccagttggcaaaacactggagaacatcaaagagaatggtctgatagatggcgacgaacagctgaatgagaattatcagaaggcgaaaattattgtggatgattttctgcgggacttcattaataaagcactgaataatacgcagatcgggaactggcgcgaactggcggatgcccttaataaagaggatgaagataacatcgagaaattgcaggataaaattcggggaatcattgtatccaaatttgaaacgtttgatctgtttagcagctattctattaagaaagatgaaaagattattgacgacgacaatgatgttgaagaagaggaactggatctgggcaagaagaccagctcatttaaatacatatttaaaaaaaacctgtttaagttagtgttgccatcctacctgaaaaccacaaaccaggacaagctgaagattattagctcgtttgataatttttcaacgtacttccgcgggttctttgaaaaccggaaaaacatttttaccaagaaaccgatctccacaagtattgcgtatcgcattgttcatgataacttcccgaaattccttgataacattcgttgttttaatgtgtggcagacggaatgcccgcaactaatcgtgaaagcagataactatctgaaaagcaaaaatgttatagcgaaagataaaagtttggcaaactattttaccgtgggcgcgtatgactatttcctgtctcagaatggtatagatttttacaacaatattataggtggactgccagcgttcgccggccatgagaaaatccaaggtctcaatgaattcatcaatcaagagtgccaaaaagacagcgagctgaaaagtaagctgaaaaaccgtcacgcgttcaaaatggcggtacttcacaaacagattctatgcattgcggacactagctatgag

SEQ ID NO: 11 - DNA, 233 bp, Artificial Sequence
  lacZ-1
tcctctggcggaaagcctacacgaagcgattttctttatggcagggtgaaacgcaggtcgccagcggcaccgcgcctttctaataagaaattatcgatgagcgtggtggttatgccgatcgcgtcacactacgtctttgacagctagctcagtcctaggtataatactagtggaatttctactcttgtagatggcggtgaaattatcgatgaatcccagaaaagacccgtccg

SEQ ID NO: 12 - DNA, 236 bp, Artificial Sequence
  lacZ-2
tcctctggcggaaagcctacacgaagcgatgtggtacacgctgtgcgaccgctacggcctgtatgtggtggatgaagcctaataagagacccacggcatggtgccaatgaatcgtctgaccgatgatccgcgctggctattgacagctagctcagtcctaggtataatactagtggaatttctactcttgtagataatattggcttcatccaccaatcccagaaaagacccgtccg

SEQ ID NO: 13 - DNA, 221 bp, Artificial Sequence
  lacZ-3
tcctctggcggaaagcctacacgaagcgatgcgaattccacgatgctgatgcgcagaactctcacagctattgccgcgaaattctggagcggcggtaattttgtatagaatttacggctagcgcttgacagctagctcagtcctaggtataatactagtggaatttctactcttgtagatatcaacattaaatgtgagcgatcccagaaaagacccgtccg

SEQ ID NO: 14 - DNA, 300 bp, Artificial Sequence
  Galk-1
tcctctggcggaaagcctgtgaacgagcatccaaaggtgtggctgtcgtcatcatcaacagtaacttcaaacgtaccctggttggcagcgaatacaacacctagggtgaacagtgcgagaccggtgcgcgtttcttccagcagccagccctgcgtgatgtcattgacagctagctcagtcctaggtataatactagtggaatttctactcttgtagatgcactgttcacgacgggtgtatcccagaaaagacccgtccgccatgccgtagcactgtgacccggccaactcccaccatttg

SEQ ID NO: 15 - DNA, 300 bp, Artificial Sequence
  Galk-2
ttccagctcgaaggcgatcgtgcacaatatgctggctgctggaagaaacgcgcaccggtttcgcactgttcaccctaggtgttgtattcgctgccaaccagggtacgcttaaagttactgttgatgatgacgacagccacacctttgggcttgacagctagctcagtcctaggtataatactagtggaatttctactcttgtagataaacgtaccctggttggcagatcccagaaaagacccgtccgccatgccgtaccgaccccaatacccgattccgacaccgtagcactgtgacccg

SEQ ID NO: 16 - DNA, 372 bp, Artificial Sequence
  Galk-3
ttccagctcgaaggcgatctaatgctcactatcgcgtggtgcacaactgatcacggtttgataatcaatcgcgcagggcagaacgaaaccgtcgttgtagtcggtgtgttcaccaatcaaattcacgcggccaggttattaaatggtgtgagtggcagggtaaccgaatgcgttggcaaacagagattgtgttttttctttcagactcatttcttacactccggattcgcgaaaatggatatcgctgactgcgcgcaaacgttgacagctagctcagtcctaggtataatactagtgtcaaaagacctttttaatttctactcttgtagatgctaccctgccactcacaccatcccagaaaagacccgtccg

SEQ ID NO: 17 - DNA, 288 bp, Artificial Sequence
  CAN1
gttcgaaacttctccgcagtgaaagataaatgatcacgttctctatggaggatggcataggtgatgaagatgaaggagaagtacagaacgctgaagtgaagagagagcttgacatattggtatgattgcccttggtggtactattggtacaggtcttttcattggtttatccacacctctgaccaacgccggatcgtcaaaagacctttttaatttctactcttgtagatcttaagctctctcttcacttctgcccgctttccaccggtggtctctagagctatgctg

SEQ ID NO: 18 - DNA, 20 bp, Artificial Sequence
  spacer for DNMT1
ctgatggtccatgtctgtta

SEQ ID NO: 19 - DNA, 1033 bp, Artificial Sequence; identical to SEQ ID NO: 10
  SD_Cpf1_1

SEQ ID NO: 20 - DNA, 3717 bp, Artificial Sequence
  CT_Cpf1
atgaacaactacgacgaattcaccaaactgtacccgatccagaaaaccatccgtttcgaactgaaaccgcagggtcgtaccatggaacacctggaaaccttcaacttcttcgaagaagaccgtgaccgtgcggaaaaatacaaaatcctgaaagaagcgatcgacgaataccacaaaaaattcatcgacgaacacctgaccaacatgtctctggactggaactctctgaaacagatctctgaaaaatactacaaatctcgtgaagaaaaagacaaaaaagttttcctgtctgaacagaaacgtatgcgtcaggaaatcgtttctgaattcaaaaaagacgaccgtttcaaagacctgttctctaaaaaactgttctctgaactgctgaaagaagaaatctacaaaaaaggtaaccaccaggaaatcgacgcgctgaaatctttcgacaaattctctggttacttcatcggtctgcacgaaaaccgtaaaaacatgtactctgacggtgacgaaatcaccgcgatctctaaccgtatcgttaacgaaaacttcccgaaattcctggacaacctgcagaaataccaggaagcgcgtaaaaaatacccggaatggatcatcaaagcggaatctgcgctggttgcgcacaacatcaaaatggacgaagttttctctctggaatacttcaacaaagttctgaaccaggaaggtatccagcgttacaacctggcgctgggtggttacgttaccaaatctggtgaaaaaatgatgggtctgaacgacgcgctgaacctggcgcaccagtctgaaaaatcttctaaaggtcgtatccacatgaccccgctgttcaaacagatcctgtctgaaaaagaatctttctcttacatcccggacgttttcaccgaagactctcagctgctgccgtctatcggtggtttcttcgcgcagatcgaaaacgacaaagacggtaacatcttcgaccgtgcgctggaactgatctcttcttacgcggaatacgacaccgaacgtatctacatccgtcaggcggacatcaaccgtgtttctaacgttatcttcggtgaatggggtaccctgggtggtctgatgcgtgaatacaaagcggactctatcaacgacatcaacctggaacgtacctgcaaaaaagttgacaaatggctggactctaaagaattcgcgctgtctgacgttctggaagcgatcaaacgtaccggtaacaacgacgcgttcaacgaatacatctctaaaatgcgtaccgcgcgtgaaaaaatcgacgcggcgcgtaaagaaatgaaattcatctctgaaaaaatctctggtgacgaagaatctatccacatcatcaaaaccctgctggactctgttcagcagttcctgcacttcttcaacctgttcaaagcgcgtcaggacatcccgctggacggtgcgttctacgcggaattcgacgaagttcactctaaactgttcgcgatcgttccgctgtacaacaaagttcgtaactacctgaccaaaaacaacctgaacaccaaaaaaatcaaactgaacttcaaaaacccgaccctggcgaacggttgggaccagaacaaagtttacgactacgcgtctctgatcttcctgcgtgacggtaactactacctgggtatcatcaacccgaaacgtaaaaaaaacatcaaattcgaacagggttctggtaacggtccgttctaccgtaaaatggtttacaaacagatcccgggtccgaacaaaaacctgccgcgtgttttcctgacctctaccaaaggtaaaaaagaatacaaaccgtctaaagaaatcatcgaaggttacgaagcggacaaacacatccgtggtgacaaattcgacctggacttctgccacaaactgatcgacttcttcaaagaatctatcgaaaaacacaaagactggtctaaattcaacttctacttctctccgaccgaatcttacggtgacatctctgaattctacctggacgttgaaaaacagggttaccgtatgcacttcgaaaacatctctgcggaaaccatcgacgaatacgttgaaaaaggtgacctgttcctgttccagatctacaacaaagacttcgttaaagcggcgaccggtaaaaaagacatgcacaccatctactggaacgcggcgttctctccggaaaacctgcaggacgttgttgttaaactgaacggtgaagcggaactgttctaccgtgacaaatctgacatcaaagaaatcgttcaccgtgaaggtgaaatcctggttaaccgtacctacaacggtcgtaccccggttccggacaaaatccacaaaaaactgaccgactaccacaacggtcgtaccaaagacctgggtgaagcgaaagaatacctggacaaagttcgttacttcaaagcgcactacgacatcaccaaagaccgtcgttacctgaacgacaaaatctacttccacgttccgctgaccctgaacttcaaagcgaacggtaaaaaaaacctgaacaaaatggttatcgaaaaattcctgtctgacgaaaaagcgcacatcatcggtatcgaccgtggtgaacgtaacctgctgtactactctatcatcgaccgttctggtaaaatcatcgaccagcagtctctgaacgttatcgacggtttcgactaccgtgaaaaactgaaccagcgtgaaatcgaaatgaaagacgcgcgtcagtcttggaacgcgatcggtaaaatcaaagacctgaaagaaggttacctgtctaaagcggttcacgaaatcaccaaaatggcgatccagtacaacgcgatcgttgttatggaagaactgaactacggtttcaaacgtggtcgtttcaaagttgaaaaacagatctaccagaaattcgaaaacatgctgatcgacaaaatgaactacctggttttcaaagacgcgccggacgaatctccgggtggtgttctgaacgcgtaccagctgaccaacccgctggaatctttcgcgaaactgggtaaacagaccggtatcctgttctacgttccggcggcgtacacctctaaaatcgacccgaccaccggtttcgttaacctgttcaacacctcttctaaaaccaacgcgcaggaacgtaaagaattcctgcagaaattcgaatctatctcttactctgcgaaagacggtggtatcttcgcgttcgcgttcgactaccgtaaattcggtacctctaaaaccgaccacaaaaacgtttggaccgcgtacaccaacggtgaacgtatgcgttacatcaaagaaaaaaaacgtaacgaactgttcgacccgtctaaagaaatcaaagaagcgctgacctcttctggtatcaaatacgacggtggtcagaacatcctgccggacatcctgcgttctaacaacaacggtctgatctacaccatgtactcttctttcatcgcggcgatccagatgcgtgtttacgacggtaaagaagactacatcatctctccgatcaaaaactctaaaggtgaattcttccgtaccgacccgaaacgtcgtgaactgccgatcgacgcggacgcgaacggtgcgtacaacatcgcgctgcgtggtgaactgaccatgcgtgcgatcgcggaaaaattcgacccggactctgaaaaaatggcgaaactggaactgaaacacaaagactggttcgaattcatgcagacccgtggtgactaa

SEQ ID NO: 21 - DNA, 3897 bp, Artificial Sequence
  TX_Cpfl
atgactaaaacatttgattcagagttttttaatttgtactcgctgcaaaaaacggtacgctttgagttaaaacccgtgggagaaaccgcgtcatttgtggaagactttaaaaacgagggcttgaaacgtgttgtgagcgaagatgaaaggcgagccgtcgattaccagaaagttaaggaaataattgacgattaccatcgggatttcattgaagaaagtttaaattattttccggaacaggtgagtaaagatgctcttgagcaggcgtttcatctttatcagaaactgaaggcagcaaaagttgaggaaagggaaaaagcgctgaaagaatgggaagcgctgcagaaaaagctacgtgaaaaagtggtgaaatgcttctcggactcgaataaagcccgcttctcaaggattgataaaaaggaactgattaaggaagacctgataaattggttggtcgcccagaatcgcgaggatgatatccctacggtcgaaacgtttaacaacttcaccacatattttaccggcttccatgagaatcgtaaaaatatttactccaaagatgatcacgccaccgctattagctttcgccttattcatgaaaatcttccaaagttttttgacaacgtgattagcttcaataagttgaaagagggtttccctgaattaaaatttgataaagtgaaagaggatttagaagtagattatgatctgaagcatgcgtttgaaatagaatatttcgttaacttcgtgacccaagcgggcatagatcagtataattatctgttaggagggaaaaccctggaggacgggacgaaaaaacaagggatgaatgagcaaattaatctgttcaaacaacagcaaacgcgagataaagcgcgtcagattcccaaactgatccccctgttcaaacagattcttagcgaaaggactgaaagccagtcctttattcctaaacaatttgaaagtgatcaggagttgttcgattcactgcagaagttacataataactgccaggataaattcaccgtgctgcaacaagccattctcggtctggcagaggcggatcttaagaaggtcttcatcaaaacctctgatttaaatgccttatctaacaccattttcgggaattacagcgtcttttccgatgcactgaacctgtataaagaaagcctgaaaacgaaaaaagcgcaggaggcttttgagaaactaccggcccattctattcacgacctcattcaatacttggaacagttcaattccagcctggacgcggaaaaacaacagagcaccgacaccgtcctgaactacttcatcaagaccgatgaattatattctcgcttcattaaatccactagcgaggctttcactcaggtgcagcctttgttcgaactggaagccctgtcatctaagcgccgcccaccggaatcggaagatgaaggggcaaaagggcaggaaggcttcgagcagatcaagcgtattaaagcttacctggatacgcttatggaagcggtacactttgcaaagccgttgtatcttgttaagggtcgtaaaatgatcgaagggctcgataaagaccagtccttttatgaagcgtttgaaatggcgtaccaagaacttgaatcgttaatcattcctatctataacaaagcgcggagctatctgtcgcggaaacctttcaaggccgataaattcaagattaattttgacaacaacacgctactgagcggatgggatgcgaacaaggaaactgctaacgcgtccattctgtttaagaaagacgggttatattaccttggaattatgccgaaaggtaagacctttctctttgactactttgtatcgagcgaggattcagagaaactgaaacagcgtcgccagaagaccgccgaagaagctctggcgcaggatggtgaaagttacttcgaaaaaattcgttataaactgttaccaggggcttcaaagatgttaccgaaagtcttttttagcaacaaaaatattggcttttacaacccgtcggatgacattttacgcattcgcaacacagcctctcacaccaaaaacgggacccctcagaaaggccactcaaaagttgagtttaacctgaatgattgtcataagatgattgatttcttcaaatcatcaattcagaaacacccggaatgggggtcttttggctttacgttttctgataccagtgattttgaagacatgagtgccttctaccgggaagtagaaaaccagggttacgtaattagctttgacaaaatcaaagagacctatatacagagccaggtggaacagggtaatctctacttattccagatttataacaaggatttctcgccctacagcaaaggcaaaccaaacctgcatactctgtactggaaagccctgtttgaagaagcgaacctgaataacgtagtggcgaagttgaacggtgaagcggaaatcttcttccgtcgtcactccattaaggcctctgataaagttgtccatccggcaaatcaggccattgataataagaatccacacacggaaaaaacgcagtcaacctttgaatatgacctcgttaaagacaaacgctacacgcaagataagttctttttccacgtcccaatcagcctcaactttaaagcacaaggggtttcaaagtttaatgataaagtcaatgggttcctcaagggcaacccggatgtcaacattataggtatagacaggggcgaacgccatctgctttactttaccgtagtgaatcagaaaggtgaaatactggttcaggaatcattaaataccttgatgtcggacaaagggcacgttaatgattaccagcagaaactggataaaaaagaacaggaacgtgatgctgcgcgtaaatcgtggaccacggttgagaacattaaagagctgaaagaggggtatctaagccatgtggtacacaaactggcgcacctcatcattaaatataacgcaatagtctgcctagaagacttgaattttggctttaaacgcggccgcttcaaagtggaaaaacaagtttatcaaaaatttgaaaaggcgcttatagataaactgaattatctggtttttaaagaaaaggaacttggtgaggtagggcactacttgacagcttatcaactgacggccccgttcgaatcattcaaaaaactgggcaaacagtctggcattctgttttacgtgccggcagattatacttcaaaaatcgatccaacaactggctttgtgaacttcctggacctgagatatcagtctgtagaaaaagctaaacaacttcttagcgattttaatgccattcgttttaacagcgttcagaattactttgaattcgaaattgactataaaaaacttactccgaaacgtaaagtcggaacccaaagtaaatgggtaatttgtacgtatggcgatgtcaggtatcagaaccgtcggaatcaaaaaggtcattgggagaccgaagaagtgaacgtgaccgaaaagctgaaggctctgttcgccagcgattcaaaaactacaactgtgatcgattacgcaaatgatgataacctgatagatgtgattttagagcaggataaagccagcttttttaaagaactgttgtggctcctgaaacttacgatgaccttacgacattccaagatcaaatcggaagatgattttattctgtcaccggtcaagaatgagcagggtgaattctatgatagtaggaaagccggcgaagtgtggccgaaagacgccgacgccaatggcgcctatcatatcgcgctcaaagggctttggaatttgcagcagattaaccagtgggaaaaaggtaaaaccctgaatctggctatcaaaaaccaggattggtttagctttatccaagagaaaccgtatcaggaatga

SEQ ID NO: 22 - DNA, 3708 bp, Artificial Sequence
  CA_Cpfl
atgcatacaggcggtcttcttagtatggacgcgaaagagttcacaggtcagtatccgttgtcgaaaacattacgattcgaacttcggcccatcggccgcacgtgggataacctggaggcctcaggctacttagcggaagaccgccatcgtgccgaatgttatcctcgtgcgaaagagttattggatgacaaccatcgtgccttcctgaatcgtgtgttgccacaaatcgatatggattggcacccgattgcggaggccttttgtaaggtacataaaaaccctggtaataaagaacttgcccaggattacaaccttcagttgtcaaagcgccgtaaggagatcagcgcatatcttcaggatgcagatggctataaaggcctgttcgcgaagcccgccttagacgaagctatgaaaattgcgaaagaaaacgggaacgaaagtgatattgaggttctcgaagcgtttaacggttttagcgtatacttcaccggttatcatgagtcacgcgagaacatttatagcgatgaggatatggtgagcgtagcctaccgaattactgaggataatttcccgcgctttgtctcaaacgctttgatctttgataaattaaacgaaagccatccggatattatctctgaagtatcgggcaatcttggagttgatgacattggtaagtactttgacgtgtcgaactataacaattttctttcccaggccggtatagatgactacaatcacattattggcggccatacaaccgaagacggactgatacaagcgtttaatgtcgtattgaacttacgtcaccaaaaagaccctggctttgaaaaaattcagttcaaacagctctacaaacaaatcctgagcgtgcgtaccagcaaaagctacatcccgaaacagtttgacaactctaaggagatggttgactgcatttgcgattatgtcagcaaaatagagaaatccgaaacagtagaacgggccctgaaactagtccgtaatatcagttctttcgacttgcgcgggatctttgtcaataaaaagaacttgcgcatactgagcaacaaactgataggagattgggacgcgatcgaaaccgcattgatgcatagttcttcatcagaaaacgataagaaaagcgtatatgatagcgcggaggcttttacgttggatgacatcttttcaagcgtgaaaaaattttctgatgcctctgccgaagatattggcaacagggcggaagacatctgtagagtgataagtgagacggccccttttatcaacgatctgcgagcggtggacctggatagcctgaacgacgatggttatgaagcggccgtctcaaaaattcgggagtcgctggagccttatatggatcttttccatgaactggaaattttctcggttggcgatgagttcccaaaatgcgcagcattttacagcgaactggaggaagtcagcgaacagctgatcgaaattattccgttattcaacaaggcgcgttcgttctgcacccggaaacgctatagcaccgataagattaaagtgaacttaaaattcccgaccttggcggacgggtgggacctgaacaaagagagagacaacaaagccgcgattctgcggaaagacggtaagtattatctggcaattctggatatgaagaaagatctgtcaagcattaggaccagcgacgaagatgaatccagcttcgaaaagatggagtataaactgttaccgagtccagtaaaaatgctgccaaagatattcgtaaaatcgaaagccgctaaggaaaaatatggcctgacagatcgtatgcttgaatgctacgataaaggtatgcataagtcgggtagtgcgtttgatcttggcttttgccatgaactcattgattattacaagcgttgtatcgcggagtacccaggctgggatgtgttcgatttcaagtttcgcgaaacttccgattatgggtccatgaaagagttcaatgaagatgtggccggagccggttactatatgagtctgagaaaaattccgtgcagcgaagtgtaccgtctgttagacgagaaatcgatttatctatttcaaatttataacaaagattactctgaaaatgcacatggtaataagaacatgcataccatgtactgggagggtctcttttccccgcaaaacctggagtcgcccgttttcaagttgtcgggtggggcagaacttttctttcgaaaatcctcaatccctaacgatgccaaaacagtacacccgaaaggctcagtgctggttccacgtaatgatgttaacggtcggcgtattccagattcaatctaccgcgaactgacacgctattttaaccgtggcgattgccgaatcagtgacgaagccaaaagttatcttgacaaggttaagactaaaaaagcggaccatgacattgtgaaagatcgccgctttaccgtggataaaatgatgttccacgtcccgattgcgatgaactttaaggcgatcagtaaaccgaacttaaacaaaaaagtcattgatggcatcattgatgatcaggatctgaaaatcattggtattgatcgtggcgagcggaacttaatttacgtcacgatggttgacagaaaagggaatatcttatatcaggattctcttaacatcctcaatggctacgactatcgtaaagctctggatgtgcgcgaatatgacaacaaggaagcgcgtcgtaactggactaaagtggagggcattcgcaaaatgaaggaaggctatctgtcattagcggtctcgaaattagcggatatgattatcgaaaataacgccatcatcgttatggaggacctgaaccacggattcaaagcgggccgctcaaagattgaaaaacaagtttatcagaaatttgagagtatgctgattaacaaactgggctatatggtgttaaaagacaagtcaattgaccaatcaggtggcgcgctgcatggataccagctggcgaaccatgttaccaccttagcatcagttggaaagcagtgtggggttatcttttatataccggcagcgttcactagtaaaatagatccgaccactggtttcgccgatctctttgccctgagtaacgttaaaaacgtagcgagcatgcgtgaattcttttccaaaatgaaatctgtcatttatgataaagctgaaggcaaattcgcattcacctttgattacttggattacaacgtgaagagcgaatgtggtcgtacgctgtggaccgtttacaccgttggtgagcgcttcacctattcccgtgtgaaccgcgaatatgtacgtaaagtccccaccgatattatctatgatgccctccagaaagcaggcattagcgtcgaaggagacttaagggacagaattgccgaaagcgatggcgatacgctgaagtctattttttacgcattcaaatacgcgctagatatgcgcgttgagaatcgcgaggaagactacattcaatcacctgtgaaaaatgcctctggggaatttttttgttcaaaaaatgctggtaaaagcctcccacaagatagcgatgcaaacggtgcatataacattgccctgaaaggtattcttcaattacgcatgctgtctgagcagtacgaccccaacgcggaatctattagacttccgctgataaccaataaagcctggctgacattcatgcagtctggcatgaagacctggaaaaattag

SEQ ID NO: 23 - DNA, 3783 bp, Artificial Sequence
  PC_Cpfl
atggatagtttgaaagatttcaccaatctgtaccctgtcagtaagacattgagatttgaattaaagcccgttggaaagactttagaaaatatcgagaaagcaggtattttgaaagaggatgagcatcgtgcagaaagttatcggagggtgaagaaaataattgatacttatcataaggtatttatcgattcttctcttgaaaatatggctaaaatgggtattgagaatgaaataaaagcaatgctccaaagtttctgcgaattgtataaaaaagatcatcgcactgagggtgaagacaaggcattagataaaattcgagcagtacttcgtggcctgattgttggggctttcactggtgtttgcggaagacgggaaaatacagtccaaaacgagaagtacgagagtttgttcaaagaaaagttgataaaagaaattttacctgattttgtgctctctactgaggctgaaagcttgcctttctctgttgaagaagctacgaggtcactgaaggagtttgatagctttacatcctactttgctggtttttacgagaatagaaagaatatatactcgacgaaacctcaatccactgccattgcttatcgtcttattcatgagaacttgccgaagttcattgataatattcttgtttttcagaagatcaaagagcctatagccaaagagctggaacatattcgtgcggacttttctgccggggggtacataaaaaaggatgagagattggaggatattttttcgttgaactattatatccacgtgttatctcaggctgggatcgaaaaatataacgcattgattgggaagattgtgacagaaggagatggagagatgaaagggctcaatgaacacatcaacctttacaaccaacaaagaggcagagaggatcggctccctctttttaggcctctttataaacagatattgagtgacagagagcaattatcatacttgcctgagagttttgaaaaagatgaggagctcctcagggctctaaaagagttctatgatcatatcgcagaagacattctcggacgtactcaacagttgatgacttctatttcagaatatgatttatctcggatatacgtaaggaacgatagccaattgactgatatatcaaaaaaaatgttgggagattggaatgctatctacatggctagagaacgagcatatgaccacgagcaggctcccaaaagaatcacggcgaaatacgagagggacaggattaaagctcttaaaggagaagagagtataagtctggcaaatcttaatagttgtattgcctttctggacaatgttagagattgccgtgtagatacttatctttccacactgggccagaaggaaggaccacatggtctatctaatctcgttgagaacgtttttgcctcataccatgaagcagagcaattgttgagctttccataccccgaagagaataatctgattcaggacaaggacaatgtggtgttaattaagaatcttctcgacaatatcagtgatctgcagaggttcttgaaacctctttggggtatgggagacgaacccgataaagatgaaagattttatggagagtataattatatccgaggagctctagatcaggtgatccctctgtacaataaggtaaggaactacctcactcggaagccttattcgaccagaaaagtaaaactcaattttgggaattctcaattgcttagtggttgggatagaaataaggaaaaggataatagctgtgtgattttgcgtaaggggcagaacttctatttggctattatgaacaataggcacaaaagaagtttcgaaaacaaggtgttgcccgagtataaggagggagaaccttacttcgaaaagatggattataaatttttgcctgatcctaataaaatgcttcctaaggtttttctttcgaaaaaaggaatagagatatacaaaccaagtccgaagcttttagaacaatatggacatggaactcacaaaaagggagatacctttagtatggatgatttgcacgaactgatcgatttcttcaaacactcaatcgaggctcatgaagattggaagcaattcggattcaaattttctgatacggctacttatgagaatgtatctagtttctatagagaagttgaggatcaggggtataagctctctttccgaaaagtttcggaatcttatgtctattcattaatagatcaaggcaagttgtatttatttcagatatacaacaaggacttttctccctgcagcaaagggacacctaatctgcataccttgtattggagaatgctttttgacgagcgcaatttggcagatgtcatatacaaactggatgggaaggctgaaatctttttccgagagaagagtttgaaaaatgatcatcccacgcatccggctggtaagcctatcaaaaagaaaagtcgacaaaaaaaaggagaggagagtctgtttgagtatgatttagtcaaggataggcactatacgatggataagttccagtttcatgtgcctattactatgaattttaaatgttctgcaggaagcaaagtcaatgatatggttaatgctcatattcgagaggcaaaggatatgcatgtcattggaattgatcgtggagaacgcaatctgctgtatatatgcgtgatagatagtcgagggacgattttggatcaaatttctctgaatacgattaacgatatagactatcatgatttattggagagtcgagacaaagaccgtcagcaggagcgccgaaactggcaaactatcgaagggatcaaggagctaaaacaaggctaccttagtcaggcggttcatcggatagccgaactgatggtggcttataaggctgtagttgctttggaggatttgaatatggggttcaaacgtgggcggcagaaagtagaaagttctgtttatcagcagtttgagaaacagctgatagataagctcaactatcttgtggacaagaagaaaaggcctgaagatattggaggattgttgagagcctatcaatttacggccccatttaagagttttaaggaaatgggaaagcaaaacggcttcttgttttatatcccggcttggaacacgagcaacatagatccgactactggatttgttaatttatttcatgcccagtatgaaaatgtagataaagcgaagagcttctttcaaaagtttgattcaattagttacaacccgaagaaagactggtttgagtttgcattcgattataaaaactttactaaaaaggctgaaggaagtcgttctatgtggatattatgcacacatggttcccgaataaagaattttagaaattcccagaagaatggtcaatgggattccgaagaattcgccttgacggaggcttttaagtctctttttgtgcgatatgagatagattataccgctgatttgaaaacagctattgtggacgaaaagcaaaaagacttcttcgtggatcttctgaagctattcaaattgacagtacagatgcgcaacagctggaaagagaaggatttggattatctaatctctcctgtagcaggggctgatggccgtttcttcgatacaagagagggaaataaaagtctgcctaaggatgcagatgccaatggagcttataatattgccctaaaaggactttgggctctacgccagattcggcaaacttcagaaggcggtaaactcaaattggcgatttccaataaggaatggctacagtttgtgcaagagagatcttacgagaaagactga

SEQ ID NO: 24 - DNA, 3792 bp, Artificial Sequence
  Control
atgaacaacggcacaaataattttcagaacttcatcgggatctcaagtttgcagaaaacgctgcgcaatgctctgatccccacggaaaccacgcaacagttcatcgtcaagaacggaataattaaagaagatgagttacgtggcgagaaccgccagattctgaaagatatcatggatgactactaccgcggattcatctctgagactctgagttctattgatgacatagattggactagcctgttcgaaaaaatggaaattcagctgaaaaatggtgataataaagataccttaattaaggaacagacagagtatcggaaagcaatccataaaaaatttgcgaacgacgatcggtttaagaacatgtttagcgccaaactgattagtgacatattacctgaatttgtcatccacaacaataattattcggcatcagagaaagaggaaaaaacccaggtgataaaattgttttcgcgctttgcgactagctttaaagattacttcaagaaccgtgcaaattgcttttcagcggacgatatttcatcaagcagctgccatcgcatcgtcaacgacaatgcagagatattcttttcaaatgcgctggtctaccgccggatcgtaaaatcgctgagcaatgacgatatcaacaaaatttcgggcgatatgaaagattcattaaaagaaatgagtctggaagaaatatattcttacgagaagtatggggaatttattacccaggaaggcattagcttctataatgatatctgtgggaaagtgaattcttttatgaacctgtattgtcagaaaaataaagaaaacaaaaatttatacaaacttcagaaacttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 25 - DNA, 3957 bp, Artificial Sequence
  FB_Cpfl
atgaccaataaattcactaaccagtattctctctctaagaccctgcgctttgaactgattccgcaggggaaaaccttggagttcattcaagaaaaaggcctcttgtctcaggataaacagagggctgaatcttaccaagaaatgaagaaaactattgataagtttcataaatatttcattgatttagccttgtctaacgccaaattaactcacttggaaacgtatctggagttatacaacaaatctgccgaaactaagaaagaacagaaatttaaagacgatttgaaaaaagtacaggacaatctgcgtaaagaaattgtcaaatccttcagtgacggcgatgctaaaagcatttttgccattctggacaaaaaagagttgattactgtggaattagaaaagtggtttgaaaacaatgagcagaaagacatctacttcgatgagaaattcaaaactttcaccacctattttacaggatttcatcaaaaccggaagaacatgtactcagtagaaccgaactccacggccattgcgtatcgtttgatccatgagaatctgcctaaatttctggagaatgcgaaagcctttgaaaagattaagcaggtcgaatcgctgcaagtgaattttcgtgaactcatgggcgaatttggtgacgaaggtctaatcttcgttaacgaactggaagaaatgtttcagattaattactacaatgacgtgctatcgcagaacggtatcacaatctacaatagtattatctcagggttcacaaaaaacgatataaaatacaaaggcctgaacgagtatatcaataactacaaccaaacaaaggacaaaaaggataggcttccgaaactgaagcagttatacaaacagattttatctgacagaatctccctgagctttctgccggatgctttcactgatgggaagcaggttctgaaagcgattttcgatttttataagattaacttactgagctacacgattgaaggtcaagaagaatctcaaaacttactgctcttgatccgtcaaaccattgaaaatctatcatcgttcgatacgcagaaaatctacctcaaaaacgatactcacctgactacgatctctcagcaggttttcggggattttagtgtattttcaacagctctgaactactggtatgaaaccaaagtcaatccgaaattcgagacggaatattctaaggccaacgaaaaaaaacgtgagattcttgataaagctaaagccgtatttactaaacaggattacttttctattgctttcctgcaggaagttttatcggagtatatcctgaccctggatcatacatctgatatcgttaaaaaacacagcagcaattgcatcgctgactatttcaaaaaccactttgtcgccaaaaaagaaaacgaaacagacaagactttcgatttcattgctaacatcaccgcaaaataccagtgtattcagggtatcttggaaaacgccgaccaatacgaagacgaactgaaacaagatcagaagctgatcgataatttaaaattcttcttagatgcaatcctggagctgctgcacttcatcaaaccgcttcatttaaagagcgagtccattaccgaaaaggacaccgccttctatgacgtttttgaaaattattatgaagccctctccttgctgactccgctgtataatatggtacgcaattacgtaacccagaaaccatattctaccgaaaaaattaaactgaactttgaaaacgcacagctgctcaacggttgggacgcgaataaagaaggtgactacctcaccaccatcctgaaaaaagatggtaactattttctggcaattatggataagaaacataataaagcattccagaaatttcctgaagggaaagaaaattacgaaaagatggtgtacaaactcttacctggagttaacaaaatgttgccgaaagtattttttagtaataagaacatcgcgtactttaacccgtccaaagaactgctggaaaattataaaaaggagacgcataagaaaggggatacctttaacctggaacattgccataccttaatagacttcttcaaggattccctgaataaacacgaggattggaaatatttcgattttcagtttagtgagaccaagtcataccaggatcttagcggcttttatcgcgaagtagaacaccaaggctataaaattaacttcaaaaacatcgacagcgaatacatcgacggtttagttaacgagggcaaactgtttctgttccagatctattcaaaggattttagcccgttctctaaaggcaaaccaaatatgcatacgttgtactggaaagcactgtttgaagagcaaaacctgcagaatgtgatttataaactgaacggccaagctgagatttttttccgtaaagcctcgattaaaccgaaaaatatcatccttcataagaagaaaataaagatcgctaaaaaacacttcatagataaaaaaaccaaaacctccgaaatagtgcctgttcaaacaattaagaacttgaatatgtactaccagggcaagatatcggaaaaggagttgactcaagacgatcttcgctatatcgataacttttcgatttttaacgaaaaaaacaagacgatcgacatcatcaaagataaacgcttcactgtagataagttccagtttcatgtgccgattactatgaacttcaaagctaccgggggtagctatatcaaccaaacggtgttggaatacctgcagaataacccggaagtcaaaatcattgggctggaccgcggagaacgtcaccttgtgtacttgaccttaatcgatcagcaaggcaacatcttaaaacaagaatcgctgaataccattacggattcaaagattagcaccccgtatcataagctgctcgataacaaggagaatgagcgcgacctggcccgtaaaaactggggcacggtggaaaacattaaggagttaaaggagggttatatttcccaggtagtgcataagatcgccactctcatgctcgaggaaaatgcgatcgttgtcatggaagacttaaacttcggatttaaacgtgggcgatttaaagtagagaaacaaatctaccagaagttagaaaaaatgctgattgacaaattaaattacttggtcctaaaagacaaacagccgcaagaattgggtggattatacaacgccctccaacttaccaataaattcgaaagttttcagaaaatgggtaaacagtcaggctttcttttttatgttcctgcgtggaacacatccaaaatcgaccctacaaccggcttcgtcaattacttctatactaaatatgaaaacgtcgacaaagcaaaagcattctttgaaaagttcgaagcaatacgttttaacgctgagaaaaaatatttcgagttcgaagtcaagaaatactcagactttaaccccaaagctgagggcacacagcaagcgtggacaatctgcacctacggcgagcgcatcgaaacgaagcgtcaaaaagatcagaataacaaatttgtttcaacacctatcaacctgaccgagaagattgaagacttcttaggtaaaaatcagattgtttatggcgacggtaactgtataaaatctcaaatagcctcaaaggatgataaagcatttttcgaaacattattatattggttcaaaatgacactgcagatgcgcaatagtgagacgcgtacagatattgattatcttatcagcccggtcatgaacgacaacggtactttttacaactccagagactatgaaaaacttgagaatccaactctccccaaagatgctgatgcgaacggtgcttatcacatcgcgaaaaaaggtctgatgctgctgaacaaaatcgaccaagccgatctgactaagaaagttgacctaagcatttcaaatcgggactggttacagtttgttcaaaagaacaaatga

SEQ ID NO: 26 - DNA, 3831 bp, Artificial Sequence
  CR_Cpfl
atgtctttcgactctttcaccaacctgtactctctgtctaaaaccctgaaattcgaaatgcgtccggttggtaacacccagaaaatgctggacaacgcgggtgttttcgaaaaagacaaactgatccagaaaaaatacggtaaaaccaaaccgtacttcgaccgtctgcaccgtgaattcatcgaagaagcgctgaccggtgttgaactgatcggtctggacgaaaacttccgtaccctggttgactggcagaaagacaaaaaaaacaacgttgcgatgaaagcgtacgaaaactctctgcagcgtctgcgtaccgaaatcggtaaaatcttcaacctgaaagcggaagactgggttaaaaacaaatacccgatcctgggtctgaaaaacaaaaacaccgacatcctgttcgaagaagcggttttcggtatcctgaaagcgcgttacggtgaagaaaaagacaccttcatcgaagttgaagaaatcgacaaaaccggtaaatctaaaatcaaccagatctctatcttcgactcttggaaaggtttcaccggttacttcaaaaaattcttcgaaacccgtaaaaacttctacaaaaacgacggtacctctaccgcgatcgcgacccgtatcatcgaccagaacctgaaacgtttcatcgacaacctgtctatcgttgaatctgttcgtcagaaagttgacctggcggaaaccgaaaaatctttctctatctctctgtctcagttcttctctatcgacttctacaacaaatgcctgctgcaggacggtatcgactactacaacaaaatcatcggtggtgaaaccctgaaaaacggtgaaaaactgatcggtctgaacgaactgatcaaccagtaccgtcagaacaacaaagaccagaaaatcccgttcttcaaactgctggacaaacagatcctgtctgaaaaaatcctgttcctggacgaaatcaaaaacgacaccgaactgatcgaagcgctgtctcagttcgcgaaaaccgcggaagaaaaaaccaaaatcgttaaaaaactgttcgcggacttcgttgaaaacaactctaaatacgacctggcgcagatctacatctctcaggaagcgttcaacaccatctctaacaaatggacctctgaaaccgaaaccttcgcgaaatacctgttcgaagcgatgaaatctggtaaactggcgaaatacgaaaaaaaagacaactcttacaaattcccggacttcatcgcgctgtctcagatgaaatctgcgctgctgtctatctctctggaaggtcacttctggaaagaaaaatactacaaaatctctaaattccaggaaaaaaccaactgggaacagttcctggcgatcttcctgtacgaattcaactctctgttctctgacaaaatcaacaccaaagacggtgaaaccaaacaggttggttactacctgttcgcgaaagacctgcacaacctgatcctgtctgaacagatcgacatcccgaaagactctaaagttaccatcaaagacttcgcggactctgttctgaccatctaccagatggcgaaatacttcgcggttgaaaaaaaacgtgcgtggctggcggaatacgaactggactctttctacacccagccggacaccggttacctgcagttctacgacaacgcgtacgaagacatcgttcaggtttacaacaaactgcgtaactacctgaccaaaaaaccgtactctgaagaaaaatggaaactgaacttcgaaaactctaccctggcgaacggttgggacaaaaacaaagaatctgacaactctgcggttatcctgcagaaaggtggtaaatactacctgggtctgatcaccaaaggtcacaacaaaatcttcgacgaccgtttccaggaaaaattcatcgttggtatcgaaggtggtaaatacgaaaaaatcgtttacaaattcttcccggaccaggcgaaaatgttcccgaaagtttgcttctctgcgaaaggtctggaattcttccgtccgtctgaagaaatcctgcgtatctacaacaacgcggaattcaaaaaaggtgaaacctactctatcgactctatgcagaaactgatcgacttctacaaagactgcctgaccaaatacgaaggttgggcgtgctacaccttccgtcacctgaaaccgaccgaagaataccagaacaacatcggtgaattcttccgtgacgttgcggaagacggttaccgtatcgacttccagggtatctctgaccagtacatccacgaaaaaaacgaaaaaggtgaactgcacctgttcgaaatccacaacaaagactggaacctggacaaagcgcgtgacggtaaatctaaaaccacccagaaaaacctgcacaccctgtacttcgaatctctgttctctaacgacaacgttgttcagaacttcccgatcaaactgaacggtcaggcggaaatcttctaccgtccgaaaaccgaaaaagacaaactggaatctaaaaaagacaaaaaaggtaacaaagttatcgaccacaaacgttactctgaaaacaaaatcttcttccacgttccgctgaccctgaaccgtaccaaaaacgactcttaccgtttcaacgcgcagatcaacaacttcctggcgaacaacaaagacatcaacatcatcggtgttgaccgtggtgaaaaacacctggtttactactctgttatcacccaggcgtctgacatcctggaatctggttctctgaacgaactgaacggtgttaactacgcggaaaaactgggtaaaaaagcggaaaaccgtgaacaggcgcgtcgtgactggcaggacgttcagggtatcaaagacctgaaaaaaggttacatctctcaggttgttcgtaaactggcggacctggcgatcaaacacaacgcgatcatcatcctggaagacctgaacatgcgtttcaaacaggttcgtggtggtatcgaaaaatctatctaccagcagctggaaaaagcgctgatcgacaaactgtctttcctggttgacaaaggtgaaaaaaacccggaacaggcgggtcacctgctgaaagcgtaccagctgtctgcgccgttcgaaaccttccagaaaatgggtaaacagaccggtatcatcttctacacccaggcgtcttacacctctaaatctgacccggttaccggttggcgtccgcacctgtacctgaaatacttctctgcgaaaaaagcgaaagacgacatcgcgaaattcaccaaaatcgaattcgttaacgaccgtttcgaactgacctacgacatcaaagacttccagcaggcgaaagaatacccgaacaaaaccgtttggaaagtttgctctaacgttgaacgtttccgttgggacaaaaacctgaaccagaacaaaggtggttacacccactacaccaacatcaccgaaaacatccaggaactgttcaccaaatacggtatcgacatcaccaaagacctgctgacccagatctctaccatcgacgaaaaacagaacacctctttcttccgtgacttcatcttctacttcaacctgatctgccagatccgtaacaccgacgactctgaaatcgcgaaaaaaaacggtaaagacgacttcatcctgtctccggttgaaccgttcttcgactctcgtaaagacaacggtaacaaactgccggaaaacggtgacgacaacggtgcgtacaacatcgcgcgtaaaggtatcgttatcctgaacaaaatctctcagtactctgaaaaaaacgaaaactgcgaaaaaatgaaatggggtgacctgtacgtttctaacatcgactgggacaacttcgtt

SEQ ID NO: 27 - DNA, 3921 bp, Artificial Sequence
  SC_Cpfl
atgacccagttcgaaggtttcaccaacctgtaccaggtttctaaaaccctgcgtttcgaactgatcccgcagggtaaaaccctgaaacacatccaggaacagggtttcatcgaagaagacaaagcgcgtaacgaccactacaaagaactgaaaccgatcatcgaccgtatctacaaaacctacgcggaccagtgcctgcagctggttcagctggactgggaaaacctgtctgcggcgatcgactcttaccgtaaagaaaaaaccgaagaaacccgtaacgcgctgatcgaagaacaggcgacctaccgtaacgcgatccacgactacttcatcggtcgtaccgacaacctgaccgacgcgatcaacaaacgtcacgcggaaatctacaaaggtctgttcaaagcggaactgttcaacggtaaagttctgaaacagctgggtaccgttaccaccaccgaacacgaaaacgcgctgctgcgttctttcgacaaattcaccacctacttctctggtttctacgaaaaccgtaaaaacgttttctctgcggaagacatctctaccgcgatcccgcaccgtatcgttcaggacaacttcccgaaattcaaagaaaactgccacatcttcacccgtctgatcaccgcggttccgtctctgcgtgaacacttcgaaaacgttaaaaaagcgatcggtatcttcgtttctacctctatcgaagaagttttctctttcccgttctacaaccagctgctgacccagacccagatcgacctgtacaaccagctgctgggtggtatctctcgtgaagcgggtaccgaaaaaatcaaaggtctgaacgaagttctgaacctggcgatccagaaaaacgacgaaaccgcgcacatcatcgcgtctctgccgcaccgtttcatcccgctgttcaaacagatcctgtctgaccgtaacaccctgtctttcatcctggaagaattcaaatctgacgaagaagttatccagtctttctgcaaatacaaaaccctgctgcgtaacgaaaacgttctggaaaccgcggaagcgctgttcaacgaactgaactctatcgacctgacccacatcttcatctctcacaaaaaactggaaaccatctcttctgcgctgtgcgaccactgggacaccctgcgtaacgcgctgtacgaacgtcgtatctctgaactgaccggtaaaatcaccaaatctgcgaaagaaaaagttcagcgttctctgaaacacgaagacatcaacctgcaggaaatcatctctgcggcgggtaaagaactgtctgaagcgttcaaacagaaaacctctgaaatcctgtctcacgcgcacgcggcgctggaccagccgctgccgaccaccctgaaaaaacaggaagaaaaagaaatcctgaaatctcagctggactctctgctgggtctgtaccacctgctggactggttcgcggttgacgaatctaacgaagttgacccggaattctctgcgcgtctgaccggtatcaaactggaaatggaaccgtctctgtctttctacaacaaagcgcgtaactacgcgaccaaaaaaccgtactctgttgaaaaattcaaactgaacttccagatgccgaccctggcgtctggttgggacgttaacaaagaaaaaaacaacggtgcgatcctgttcgttaaaaacggtctgtactacctgggtatcatgccgaaacagaaaggtcgttacaaagcgctgtctttcgaaccgaccgaaaaaacctctgaaggtttcgacaaaatgtactacgactacttcccggacgcmcgaaaatgatcccgaaatgctctacccagctgaaagcggttaccgcgcacttccagacccacaccaccccgatcctgctgtctaacaacttcatcgaaccgctggaaatcaccaaagaaatctacgacctgaacaacccggaaaaagaaccgaaaaaattccagaccgcgtacgcgaaaaaaaccggtgaccagaaaggttaccgtgaagcgctgtgcaaatggatcgacttcacccgtgacttcctgtctaaatacaccaaaaccacctctatcgacctgtcttctctgcgtccgtcttctcagtacaaagacctgggtgaatactacgcggaactgaacccgctgctgtaccacatctctttccagcgtatcgcggaaaaagaaatcatggacgcggttgaaaccggtaaactgtacctgttccagatctacaacaaagacttcgcgaaaggtcaccacggtaaaccgaacctgcacaccctgtactggaccggtctgttctctccggaaaacctggcgaaaacctctatcaaactgaacggtcaggcggaactgttctaccgtccgaaatctcgtatgaaacgtatggcgcaccgtctgggtgaaaaaatgctgaacaaaaaactgaaagaccagaaaaccccgatcccggacaccctgtaccaggaactgtacgactacgttaaccaccgtctgtctcacgacctgtctgacgaagcgcgtgcgctgctgccgaacgttatcaccaaagaagtttctcacgaaatcatcaaagaccgtcgtttcacctctgacaaattcttcttccacgttccgatcaccctgaactaccaggcggcgaactctccgtctaaattcaaccagcgtgttaacgcgtacctgaaagaacacccggaaaccccgatcatcggtatcgaccgtggtgaacgtaacctgatctacatcaccgttatcgactctaccggtaaaatcctggaacagcgttctctgaacaccatccagcagttcgactaccagaaaaaactggacaaccgtgaaaaagaacgtgttgcgmcgtcaggcgtggtctgttgttggtaccatcaaagacctgaaacagggttacctgtctcaggttatccacgaaatcgttgacctgatgatccactaccaggcggttgttgttctggaaaacctgaacttcggtttcaaatctaaacgtaccggtatcgcggaaaaagcggtttaccagcagttcgaaaaaatgctgatcgacaaactgaactgcctggttctgaaagactacccggcggaaaaagttggtggtgttctgaacccgtaccagctgaccgaccagttcacctctttcgcgaaaatgggtacccagtctggtttcctgttctacgttccggcgccgtacacctctaaaatcgacccgctgaccggtttcgttgacccgttcgtttggaaaaccatcaaaaaccacgaatctcgtaaacacttcctggaaggtttcgacttcctgcactacgacgttaaaaccggtgacttcatcctgcacttcaaaatgaaccgtaacctgtctttccagcgtggtctgccgggtttcatgccggcgtgggacatcgttttcgaaaaaaacgaaacccagttcgacgcgaaaggtaccccgttcatcgcgggtaaacgtatcgttccggttatcgaaaaccaccgtttcaccggtcgttaccgtgacctgtacccggcgaacgaactgatcgcgctgctggaagaaaaaggtatcgttttccgtgacggttctaacatcctgccgaaactgctggaaaacgacgactctcacgcgatcgacaccatggttgcgctgatccgttctgttctgcagatgcgtaactctaacgcggcgaccggtgaagactacatcaactctccggttcgtgacctgaacggtgtttgcttcgactctcgtttccagaacccggaatggccgatggacgcggacgcgaacggtgcgtaccacatcgcgctgaaaggtcagctgctgctgaaccacctgaaagaatctaaagacctgaaactgcagaacggtatctctaaccaggactggctggcgtacatccaggaactgcgtaactag

SEQ ID NO: 28 - protein, 1282 aa, Artificial Sequence
  CU_CH9
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 29 - protein, 1283 aa, Artificial Sequence
  CU-CH5
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEAFPELKFDKVKEDLEVDYDLKHAFEIEYFVNFVTQAGIDQYNYLLGGKTLEDGTKKQGMNEQINLFKQQQTRDKARQIPKLIPLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFIITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 30 - protein, 1272 aa, Artificial Sequence
  CU-CH4
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 31 - protein, 1286 aa, Artificial Sequence
  CU-CH6
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 32 - protein, 1291 aa, Artificial Sequence
  CU-0-11
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 33 - protein, 1286 aa, Artificial Sequence
  CU_CH2
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 34 - protein, 1270 aa, Artificial Sequence
  CU_CH3
MTNKFTNQYSLSKTLRFELIPQGKTLEFIQEKGLLSQDKQRAESYQEMKKTIDKFHKYFIDLALSNAKLTHLETYLELYNKSAETKKEQKFKDDLKKVQDNLRKEIVKSFSDGDAKSIFAILDKKELITVELEKWFENNEQKDIYFDEKFKTFTTYFTGFHQNRKNMYSVEPNSTAIAYRLIHENLPKFLENAKAFEKIKQVESLQVNFRELMGEFGDEGLIFVNELEEMFQINYYNDVLSQNGITIYNSIISGFTKNDIKYKGINEYINNYNQTKDKKDRLPKLKQLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHIVIGIDRGERNLIYVSVIDTCGNIVEQKSENIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 35 - protein, 1286 aa, Artificial Sequence; identical to SEQ ID NO: 31
  CU_CH6

SEQ ID NO: 36 - protein, 1262 aa, Artificial Sequence
  CU_CH7
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

(19) United States
(12) Patent Application Publication
(10) Pub. No.: US 2021/0309980 A1
(43) Pub. Date: Oct. 7, 2021

(54) ENGINEERED CHIMERIC NUCLEIC ACID GUIDED NUCLEASE CONSTRUCTS AND USES THEREOF

(71) Applicant: THE REGENTS OF THE UNIVERSITY OF COLORADO, A BODY CORPORATE, Denver, CO (US)

(72) Inventors: Ryan T. Gill, Boulder, CO (US); Rongming Liu, Boulder, CO (US)

(21) Appl. No.: 17/212,484

(22) Filed: Mar. 25, 2021

Related U.S. Application Data
(63) Continuation of application No. PCT/US2019/054872, filed on Oct. 4, 2019.
(60) Provisional application No. 62/741,475, filed on Oct. 4, 2018.

(51) Int. Cl. C12N 9/22; C12N 15/90; C12N 15/11

(52) U.S. Cl. CPC C12N 9/22; C12N 15/902; A61K 38/00; C12N 2800/80; C12N 15/11

(57) ABSTRACT

Embodiments of the present disclosure relate to engineered chimeric nucleic acid guided nucleases for improved targeted gene editing. In certain embodiments, the engineered chimeric nucleic acid guided nucleases can be used for genome editing. In accordance with these embodiments, a targeted genome can be edited by one or more of the engineered chimeric nucleic acid guided nucleases comprising one or more nucleic acid or amino acid constructs represented by one or more of SEQ ID NO:1 to SEQ ID NO:9 or a polypeptide encoded thereof. In certain embodiments, the engineered chimeric nucleic acid guided nucleases can be used to remove, edit, and/or insert genes into a targeted genome. In other embodiments, use of these chimeras can be for producing a targeted result (e.g. removing, editing or replacing a defective gene) in a subject to reduce the onset of or prevent a condition.

### PRIORITY

This application is a continuation of PCT International Application No. PCT/US19/54872 filed Oct. 4, 2019 which claims priority to U.S. Provisional Application No. 62/741,475 filed Oct. 4, 2018. These applications are incorporated herein by reference in their entirety for all purposes.

### STATEMENT REGARDING GOVERNMENT FUNDING

This invention was made with government support under grant number DE-SC0018368 awarded by the U.S. Department of Energy. The government has certain rights in the invention.

### SEQUENCE LISTING STATEMENT

The instant application contains a Sequence Listing which has been submitted via ASCII copy created on Oct. 4, 2019 named ‘CU4819B_Final_for_ST25.txt’ 108 kilobytes in size having 36 sequences.

### FIELD

Embodiments of the present disclosure relate to engineered chimeric nucleic acid guided nucleases for improved targeted gene editing. In certain embodiments, the engineered chimeric nucleic acid guided nucleases can be used for genome editing. In accordance with these embodiments, a targeted genome can be edited by one or more of the engineered chimeric nucleic acid guided nucleases comprising one or more constructs represented by one or more nucleic acid sequences of SEQ ID NO:1 to SEQ ID NO:9 or amino acid sequences SEQ ID NO:28 to SEQ ID NO:36 or combinations thereof. In certain embodiments, the engineered chimeric nucleic acid guided nucleases can be used to remove and/or insert and/or edit genes in a targeted genome. In other embodiments, use of these chimeras can be for producing a targeted result (e.g. removing or replacing a defective gene) in a subject to reduce the onset of, ameliorate or prevent a condition.

### BACKGROUND

CRISPR is an abbreviation of Clustered Regularly Interspaced Short Palindromic Repeats. In a palindromic repeat, the sequence of nucleotides is the same in both directions. Each of these palindromic repetitions is followed by short segments of spacer DNA. Small clusters of Cas (CRISPR-associated system) genes are located next to CRISPR sequences. The CRISPR/Cas system is a prokaryotic immune system that can confer resistance to foreign genetic elements such as those present within plasmids and phages providing the prokaryote a form of acquired immunity. RNA harboring a spacer sequence assists Cas (CRISPR-associated) proteins to recognize and cut exogenous DNA. CRISPR sequences are found in approximately 50% of bacterial genomes and nearly 90% of sequenced archaea has selected for efficient and robust metabolic and regulatory networks that prevent unnecessary metabolite biosynthesis and optimally distribute resources to maximize overall cellular fitness. The complexity of these networks with limited approaches to understand their structure and function and the ability to re-program cellular networks to modify these systems for a diverse range of applications has complicated advances in this space. Certain approaches to re-program cellular networks are directed to modifying single genes of complex pathways but as a consequence of modifying single genes, unwanted modifications to the genes or other genes can result, getting in the way of identifying changes necessary to achieve a particular endpoint as well as complicating the endpoint sought by the modification.

CRISPR-Cas driven genome editing and engineering has dramatically impacted biology and biotechnology in general. CRISPR-Cas editing systems require a polynucleotide guided nuclease, a guide polynucleotide (e.g. a guide RNA (gRNA)) that directs by homology the nuclease to cut a specific region of the genome, and, optionally, a donor DNA cassette that can be used to repair the cut dsDNA and thereby incorporate programmable edits at the site of interest. The earliest demonstrations and applications of CRISPR-Cas editing used Cas9 nucleases and associated gRNA. These systems have been used for gene editing in a broad range of species encompassing bacteria to higher order mammalian systems such as animals and in certain cases, humans. It is well established, however, that key editing parameters such as protospacer adjacent motif (PAM) specificity, editing efficiency, and off-target rates, among others, are species, loci, and nuclease dependent. There is increasing interest in identifying and rapidly characterizing novel nuclease systems that can be exploited to broaden and improve overall editing capabilities.

One version of the CRISPR/Cas system, CRISPR/Cas9, has been modified to provide useful tools for editing genomes. By delivering the Cas9 nuclease complexed with a synthetic guide RNA (gRNA) into a cell, the cell's genome can be cut/edited at a predetermined location, allowing existing genes to be removed and/or new ones added. These systems are useful but have some important limitations regarding efficiency and accuracy of targeted editing, imprecise editing complications, as well as, impediments when used for commercially relevant situations such as gene replacement. Therefore, a need exists for improved nucleic acid guided nuclease constructs for directed and accurate editing with improved efficiency.

### SUMMARY

Embodiments of the present disclosure relate to engineered chimeric nucleic acid guided nucleases having a nucleic acid sequence represented by SEQ ID NO: 1 to SEQ ID NO:9 or an amino acid sequence represented by or amino acid sequences represented by SEQ ID NO:28 to SEQ ID NO:36, or chimeric constructs of at least about 80%, about 85%, about 90% or about 95% or about 99% or more identity thereof, for improved targeted gene editing. In certain embodiments, the engineered chimeric nucleic acid guided nucleases can be used for genome editing. In other embodiments combinations of these engineered chimeric nucleic acid guided nucleases can be used to produce optimal editing results. In accordance with these embodiments, one or more targeted genomes can be edited by one or more of the engineered chimeric nucleic acid guided nucleases to remove, edit and/or insert genes into the targeted genome providing methods for producing a targeted result (e.g. removing and/or replacing a defective gene). In some embodiments, engineered chimeric nucleic acid guided nucleases disclosed herein can have reduced off-targeting rates compared to a control, wild-type Cas12a not represented by chimeras contemplated herein.

Embodiments of the present disclosure relate to compositions and methods of use of Cas12a chimeras represented by one or more of nucleic acid sequences of SEQ ID NOs: 1 to 9 or amino acid sequences represented by SEQ ID NO:28 to SEQ ID NO:36 with at least a sequence having about 80%, about 85%, about 90% or about 95% or more sequence identity thereof for use in targeting genome editing. In other embodiments, the engineered chimeric nucleic acid guided nucleases can further include one or more mutations, one or more manipulations or modifications that increase gene editing efficiency or accuracy. In some embodiments, the one or more mutations can include one or more point mutation(s), single nucleotide polymorphism (SNP), an insertion or a deletion of two or more nucleotides or other mutation to increase editing efficiency or accuracy of the chimeric constructs and/or reduce off-targeting rates compared to a control, wild-type Cas12a nuclease.

In certain embodiments, chimeric constructs disclosed herein were obtained starting with two Cas12 as in order to generate a chimera by use of cross-over recombination technologies. In other embodiments, chimeric constructs disclosed herein were obtained using three different Cas12 as to generate a chimera by use of cross-over recombination technologies. In certain embodiments, chimeric Cas12a constructs can include constructs with reduced off-targeting rates and/or improved editing functions compared to a control or wild-type Cas12a nuclease.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-1C illustrates a schematic diagram for creating and testing certain designer chimeric constructs (1A), chimeric recombinations (1B) designed by methods and testing by editing efficiency (1C) compared to a positive control of some embodiments disclosed herein.

FIG. 2 illustrates a schematic of components of use in genetic editing of some embodiments disclosed herein.

FIG. 3 illustrates exemplary screening of binding and cleavage of an altered PAM recognition sequences for a Cas12a-like chimeric nuclease construct compared to a control of some embodiments disclosed herein.

FIGS. 4A and 4B represent gene editing efficiencies (4B) of a Cas12a-like chimeric nuclease construct disclosed herein compared with a control when gRNA is conserved (WT) or mutated (4A) to test off-targeting rates.

FIGS. 5A-5D represents plots of various Cas12a-like chimeric nuclease constructs off-targeting rates compared to a control using wild-type and altered gRNA sequences in certain embodiments disclosed herein.

FIGS. 6A-6C represents a schematic illustration of genetic editing (6A) and histogram plots (6B and 6C) of various Cas12a chimeras compared to a control demonstrating editing efficiency relative to induction time of each variant and the control in certain embodiments disclosed herein.

FIG. 7 represents Cas12a-type chimera library editing and transformation efficiencies of the Cas12a like nucleases used in certain embodiments disclosed herein.

FIGS. 8A-8I 8A is an illustration of a schematic for testing editing efficiency of certain constructs created by methods disclosed herein. 8B represents a histogram plot of cutting efficiency of chimeric Cas12a-like proteins using 6 different gRNA plasmids. 8C represents a plot of percent editing efficiency of chimera library variants with different gRNAs of galK1, galK2, lacZ1, and lacZ2. 8D is a schematic representation of a Cas12a-type with reduced activity (dCas12a) in a protein binding assay. 8E-8F represents plasmid systems where one plasmid expresses dCas12a (Cas12a with reduced activity) using an inducible promoter; a second plasmid expresses a single crRNA a test gene; and a third plasmid expresses the a resistance protein using a constitutive promoter containing a fully complementary (on-target) crRNA binding site as well as an encoded enzyme making the cells sensitive to an agent. 8E and 8F represent cutting efficiency of some chimeric Cas12a like nucleases with different induction times using different gRNAs. (8E) galK_1 and (8F) galK_2. 8G is an illustration of an inducible system for testing chimeric nucleases disclosed herein. Three additional plasmid systems were constructed for genome editing: where one plasmid expresses a Cas12a like protein using an inducible promoter; a second plasmid bacterial test proteins using a temperature-inducible promoter; and a third plasmid expresses a single crRNA (with a promoter) targeting a tester gene with homology arm (HM) containing a test protein-inactivating mutation as a template for recombineering. (8H and 8I) The editing efficiency of chimeric Cas12a-like nucleases with different gene induction times with different gRNAs is plotted in (8H) galK_1 and (8I) galK_2.

FIGS. 9A-9F represents specificity detection of chimeric Cas12a-like variants and enrichment scoring of each PAM site using different guide RNAs. (9A-9F) Round 1 is illustrated of enrichment scores for two rounds of PAM scans. The enrichment score is the frequency change (log 2) of each PAM using different gRNA plasmids (on-targeting and non-targeting gRNAs).

FIG. 9G illustrates an off-target assay for chimeric Cas12a-type variants. 9G represents an individual off-target assay. Nine different off-target spacers were designed as illustrated to test editing efficiency and target recognition, of which 3 were substitutions, 3 were deletions, and 3 were insertions.

FIGS. 10A-10F illustrates in (10A) a plasmid expressing the M44 (or control) nuclease (with T7 promoter), a single crRNA (with U6 promoter), and GFP were constructed. 10B is a photographic representation of the mammalian cells after transfection. Micrographs were taken under cool white light (left) or fluorescent light (right). An assay was performed as known in the art on cells expressing GFP and isolated by fluorescence activated cell sorting. In this example, ‘Untreated’ as labeled means the PCR products without T7 endonuclease treatment; while ‘Treated’ means the PCR products with T7 endonuclease treatment (10C). 10D is a graphic representation of an indel rate of control versus the chimeric nuclease of certain embodiments disclosed herein. 10F is a graphic illustration of editing efficiency of control and the tested chimera Cas12a-like nuclease, of certain embodiments disclosed herein.

FIG. 11 is an exemplary graph illustrating distribution of functional chimera Cas12a-like nucleases of certain embodiments disclosed herein.

FIG. 12 illustrates a color screening of control versus a chimera Cas12a-like nuclease (e.g. M44) with different gRNAs of certain embodiments disclosed herein.

FIGS. 13A-13D illustrate exemplary histogram plots that represent transformation efficiency of different Cas12a-like chimera variants using different gRNA of certain embodiments disclosed herein. The gRNA used in the test were (13A) galK1 (13B) galK2 (13C) lacZ1 and (13D) lacZ2.

FIGS. 14A-14C illustrate genome editing test in the different genomic positions for chimera library variants. 14A illustrates a schematic of targeted genomic positions. 14B illustrates representative plates for colorimetric screening of targeted protein activity with chimera Cas12a-like nuclease variants in different genomic position. 14C illustrates editing efficiency of chimera library variants in different genomic positions of certain embodiments disclosed herein.

FIG. 15 represents a histogram plot of binding efficiency of dCas12a-like chimera nucleases using different guide RNAs of certain embodiments disclosed herein.

FIGS. 16A-16E represent (A) a schematic illustration of an exemplary plasmid construct of some embodiments disclosed herein and (B)-(E) represent histogram plots illustrating cutting efficiency assessed by individual verification of unknown PAMs using different nucleases including chimera Cas12a-like nucleases (B)ATTC (C) ATTA (D) GTTA and (E) CCTC.

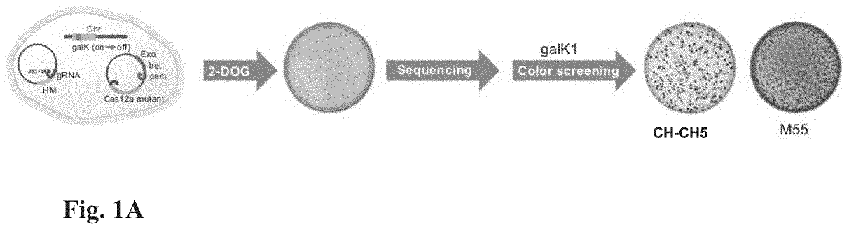

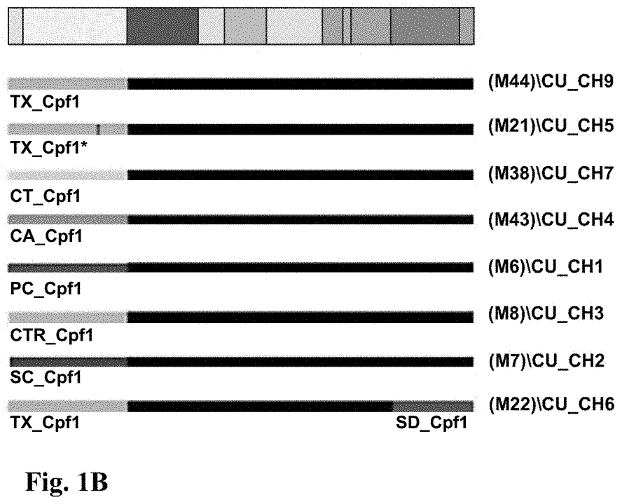

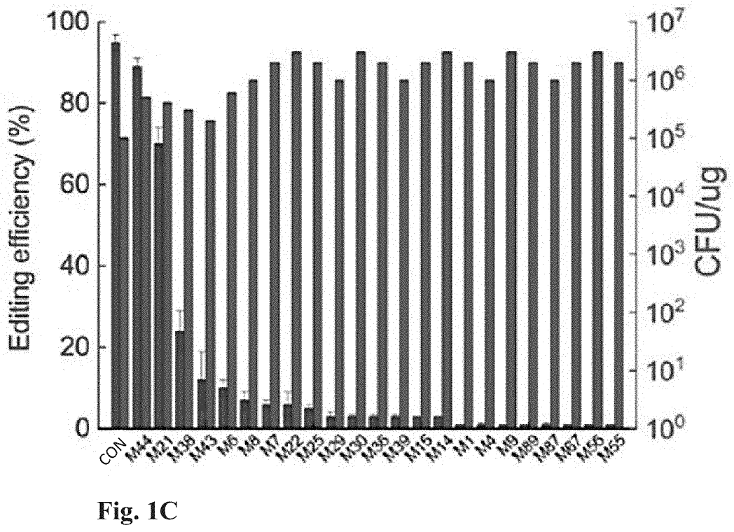

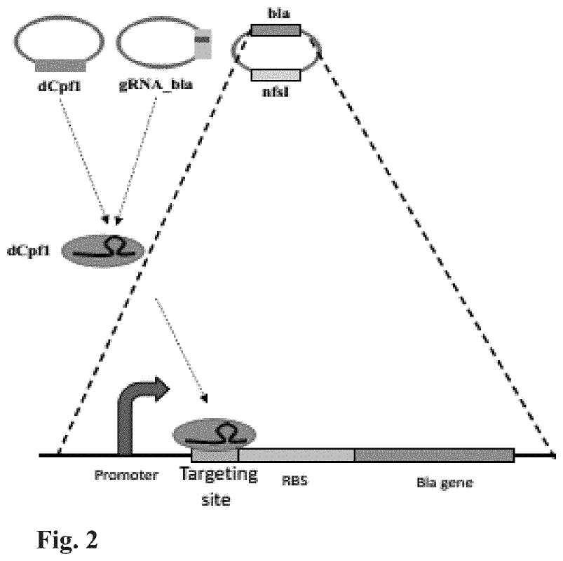

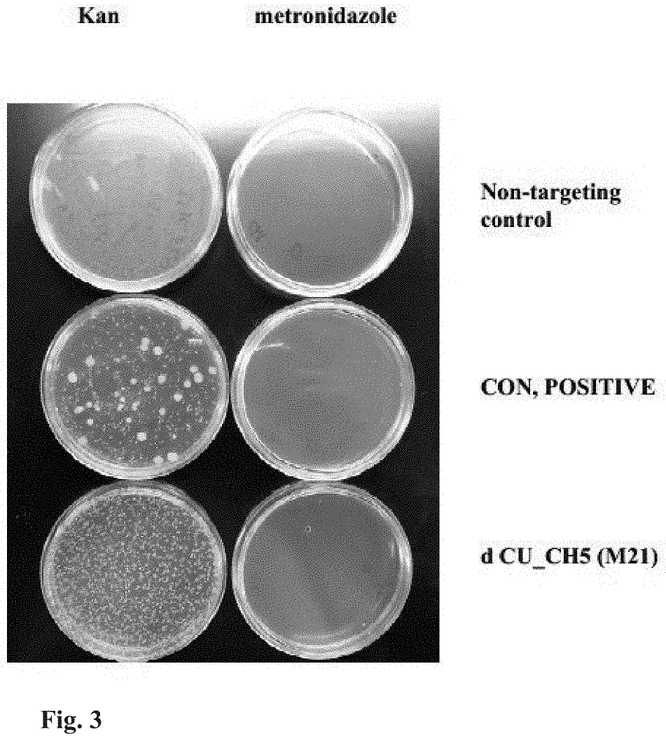

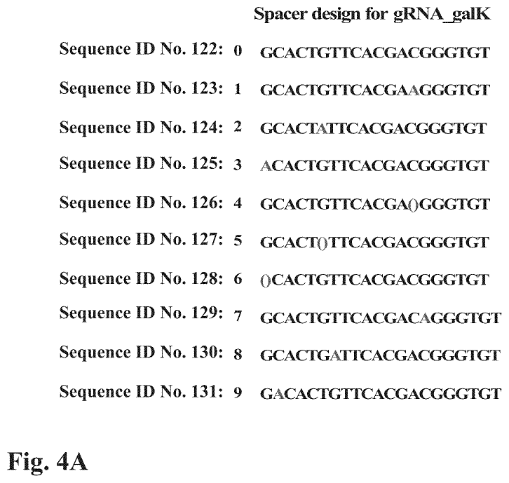

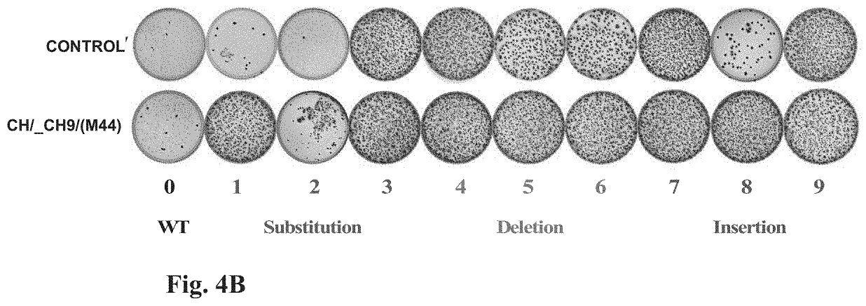

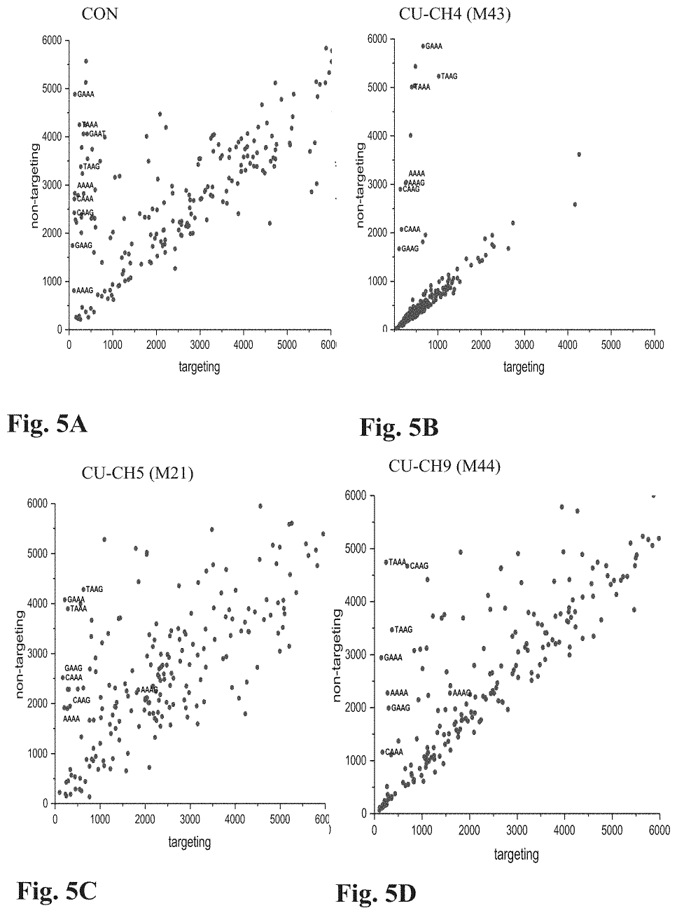

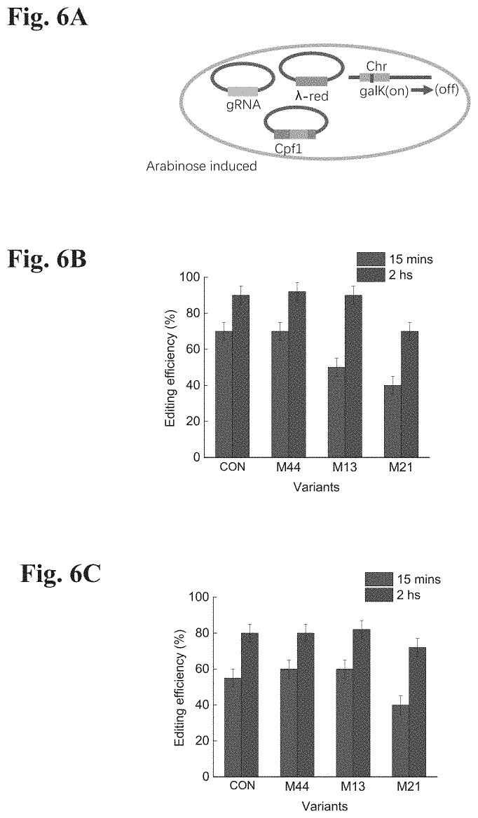

### DETAILED DESCRIPTION

In the following sections, various exemplary constructs are described in order to detail various embodiments of the disclosure. It will be obvious to one of skill in the relevant art that practicing the various embodiments does not require the employment of all or even some of the details outlined herein, but rather that combinations, concentrations, times and other details may be modified through routine experimentation. In some cases, well-known methods or components have not been included in the description.

As disclosed herein “modulating” and “manipulating” of genome editing can mean an increase, a decrease, upregulation, downregulation, induction, a change in editing activity, a change in binding, a change cleavage or the like, of one or more of targeted genes or gene clusters of certain embodiments disclosed herein.

In certain embodiments of the present disclosure, there can be employed conventional molecular biology, microbiology, and recombinant DNA techniques within the skill of the art. Such techniques are explained fully in the literature and understood by those of skill in the art.

In certain embodiments of this disclosure, primers used for example, for sequencing and sample preparation per conventional techniques can include sequencing primers and amplification primers. In some embodiments, plasmids and oligomers can be used per conventional techniques and can include synthesized oligomers, oligomer cassettes.

In certain embodiments of the present disclosure, there can be employed conventional molecular biology, microbiology, and recombinant DNA techniques within the skill of the art. Such techniques are explained fully in the literature and understood by those of skill in the art.

In certain embodiments of this disclosure, primers used for sequencing and sample preparation per conventional techniques can include sequencing primers and amplification primers. In some embodiments, plasmids and oligomers used per conventional techniques can include synthesized oligomers, oligomer cassettes or similar.

In certain embodiments, engineered chimeric nucleic acid guided nucleases disclosed herein can be used to target and edit a gene of interest having unique editing capabilities compared to control nucleic acid guided nucleases; for example, altered PAM preferences and off-target editing rates.

In accordance with these embodiments, it is known that Cas12a is a novel single RNA-guided CRISPR/Cas endonuclease capable of genome editing having differing features when compared to Cas9. In certain embodiments, a Cas12a-based system allow fast and reliable introduction of donor DNA into a genome. In addition, Cas12a broadens genome editing. CRISPR/Cas12a genome editing has been evaluated in human cells as well as other organisms including plants. Several features of the CRISPR/Cas12a system are different when compared to CRISPR/Cas9.

For example, Cas12a recognizes T-rich protospacer adjacent motif (PAM) sequences (e.g. 5′-TTTN-3′ (AsCas12a, LbCas12a) and 5′-TTN-3′ (FnCas12a); whereas, the comparable sequence for SpCas9 is NGG. The PAM sequence of Cas12a is located at the 5′ end of the target DNA sequence, where it is at the 3′ end for Cas9. In addition, Cas12a is capable of cleaving DNA distal to its PAM around the +18/+23 position of the protospacer. This cleavage creates a staggered DNA overhang (e.g. sticky ends), whereas Cas9 cleaves close to its PAM after the 3′ position of the protospacer at both strands and creates blunt ends. In certain methods, creating altered recognition of Cas12a nucleases can provide an improvement over Cas9 in part due to the creation of sticky ends instead of blunt end cleavages. Further, Cas12a is guided by a single crRNA and does not require a tracrRNA, resulting in a shorter gRNA sequence than the sgRNA used by Cas9.

In some embodiments, systems for using engineered chimeric nucleic acid guided nuclease constructs disclosed herein are combined with guide RNAs (gRNA) where the gRNA targets a specific region of a gene opening up the double-stranded DNA region to allow the engineered chimeric nucleic acid guided nuclease constructs to cut the DNA further facilitating insertions and/or deletions. Guide RNAs of the instant disclosure can contain a 4- to 18-nt anchor sequence, which is the opposite of the sequence immediately downstream of a targeted editing site on unedited transcripts. Guide RNAs hybridize with the preedited RNA, but are mismatched at the editing site. 5′ of the mismatch between the guide RNA and the unedited premessenger RNAs, the RNA backbone, is cleaved by an endonuclease. In certain embodiments, U is added by the enzyme terminal ribonucleotide transferase or deleted by an exonuclease as directed by the guide RNA template. The free ends of the corrected RNA can be ligated by an RNA ligase enzyme, for example.

In certain embodiments, engineered chimeric nucleic acid guided nuclease constructs disclosed herein and gRNA can be delivered to a cell in a variety of forms (e.g., plasmid DNA, mRNA, protein, lentivirus or similar) and using a variety of methods (e.g., electroporation, lipofection, calcium phosphate transfection, transduction). In certain embodiments, chemical modifications to gRNAs contemplated herein can be used to increase gRNA stability in order to obtain higher indel frequency in human cells, for example.

It is also known that Cas12a displays additional ribonuclease activity that functions in crRNA processing. This feature may lead to simplified multiplex genome editing. Cas12a is used as an editing tool for different species (e.g. S. cerevisiae), allowing the use of an alternative PAM sequence compared with the one recognized by CRISPR/Cas9. It also provides an alternative system for multiplex genome editing as compared with Cas9-based multiplex approaches for yeast and can be used as an improved system in mammalian gene editing.

In certain embodiments, designer engineered chimeric nucleic acid guided nuclease constructs of embodiments disclosed herein enable altered and/or improved CRISPR-Cas editing. In other embodiments, activity of these novel designer constructs have been analyzed in bacteria (e.g. E. coli) and confirmed in yeast and in human cells.

In some embodiments, engineered chimeric nucleic acid guided nuclease constructs of certain embodiments disclosed herein can include, but are not limited to, SEQ ID NO:1 to SEQ ID NO:9.

CU-CH2: SEQ ID NO: 1:
atgacccagttcgaaggtttcaccaacctgtaccaggtttctaaaaccctgcgtttcgaactgatcccgcagggtaaaaccctgaaaca
catccaggaacagggtttcatcgaagaagacaaagcgcgtaacgaccactacaaagaactgaaaccgatcatcgaccgtatctaca
aaacctacgcggaccagtgcctgcagctggttcagctggactgggaaaacctgtctgcggcgatcgactcttaccgtaaagaaaaa
accgaagaaacccgtaacgcgctgatcgaagaacaggcgacctaccgtaacgcgatccacgactacttcatcggtcgtaccgaca
acctgaccgacgcgatcaacaaacgtcacgcggaaatctacaaaggtctgttcaaagcggaactgttcaacggtaaagttctgaaac
agctgggtaccgttaccaccaccgaacacgaaaacgcgctgctgcgttctttcgacaaattcaccacctacttctctggtttctacgaaa
accgtaaaaacgttttctctgcggaagacatctctaccgcgatcccgcaccgtatcgttcaggacaacttcccgaaattcaaagaaaac
tgccacatcttcacccgtctgatcaccgcggttccgtctctgcgtgaacacttcgaaaacgttaaaaaagcgatcggtatcttcgtacta
cctctatcgaagaagttttctctttcccgttctacaaccagctgctgacccagacccagatcgacctgtacaaccagctgctgggtggta
tctctcgtgaagcgggtaccgaaaaaatcaaaggtctgaacgaagttctgaacctggcgatccagaaaaacgacgaaaccgcgcac
atcatcgcgtctctgccgcaccgtttcatcccgCTTCACAAACAGATTCTATGCATTGCGGACACTA
GCTATGAGGTCCCGTATAAATTTGAAAGTGACGAGGAAGTGTACCAATCAGTTA
ACGGCTTCCTTGATAACATTAGCAGCAAACATATAGTCGAAAGATTACGCAAAA
TCGGCGATAACTATAACGGCTACAACCTGGATAAAATTTATATCGTGTCCAAAT
TTTACGAGAGCGTTAGCCAAAAAACCTACCGCGACTGGGAAACAATTAATACCG
CCCTCGAAATTCATTACAATAATATCTTGCCGGGTAACGGTAAAAGTAAAGCCG
ACAAAGTAAAAAAAGCGGTTAAGAATGATTTACAGAAATCCATCACCGAAATA
AATGAACTAGTGTCAAACTATAAGCTGTGCAGTGACGACAACATCAAAGCGGA
GACTTATATACATGAGATTAGCCATATCTTGAATAACTTTGAAGCACAGGAATT
GAAATACAATCCGGAAATTCACCTAGTTGAATCCGAGCTCAAAGCGAGTGAGCT
TAAAAACGTGCTGGACGTGATCATGAATGCGTTTCATTGGTGTTCGGTTTTTATG
ACTGAGGAACTTGTTGATAAAGACAACAATTTTTATGCGGAACTGGAGGAGATT
TACGATGAAATTTATCCAGTAATTAGTCTGTACAACCTGGTTCGTAACTACGTTA
CCCAGAAACCGTACAGCACGAAAAAGATTAAATTGAACTTTGGAATACCGACGT
TAGCAGACGGTTGGTCAAAGTCCAAAGAGTATTCTAATAACGCTATCATACTGA
TGCGCGACAATCTGTATTATCTGGGCATCTTTAATGCGAAGAATAAACCGGACA
AGAAGATTATCGAGGGTAATACGTCAGAAAATAAGGGTGACTACAAAAAGATG
ATTTATAATTTGCTCCCGGGTCCCAACAAAATGATCCCGAAAGTTTTCTTGAGCA
GCAAGACGGGGGTGGAAACGTATAAACCGAGCGCCTATATCCTAGAGGGGTAT
AAACAGAATAAACATATCAAGTCTTCAAAAGACTTTGATATCACTTTCTGTCAT
GATCTGATCGACTACTTCAAAAACTGTATTGCAATTCATCCCGAGTGGAAAAAC
TTCGGTTTTGATTTTAGCGACACCAGTACTTATGAAGACATTTCCGGGTTTTATC
GTGAGGTAGAGTTACAAGGTTACAAGATTGATTGGACATACATTAGCGAAAAA
GACATTGATCTGCTGCAGGAAAAAGGTCAACTGTATCTGTTCCAGATATATAAC
AAAGATTTTTCGAAAAAATCAACCGGGAATGACAACCTTCACACCATGTACCTG
AAAAATCTTTTCTCAGAAGAAAATCTTAAGGATATCGTCCTGAAACTTAACGGC
GAAGCGGAAATCTTCTTCAGGAAGAGCAGCATAAAGAACCCAATCATTCATAA
AAAAGGCTCGATTTTAGTCAACCGTACCTACGAAGCAGAAGAAAAAGACCAGT
TTGGCAACATTCAAATTGTGCGTAAAAATATTCCGGAAAACATTTATCAGGAGC
TGTACAAATACTTCAACGATAAAAGCGACAAAGAGCTGTCTGATGAAGCAGCC
AAACTGAAGAATGTAGTGGGACACCACGAGGCAGCGACGAATATAGTCAAGGA
CTATCGCTACACGTATGATAAATACTTCCTTCATATGCCTATTACGATCAATTTC
AAAGCCAATAAAACGGGTTTTATTAATGATAGGATCTTACAGTATATCGCTAAA
GAAAAAGACTTACATGTGATCGGCATTGATCGGGGCGAGCGTAACCTGATCTAC
GTGTCCGTGATTGATACTTGTGGTAATATAGTTGAACAGAAAAGCTTTAACATT
GTAAACGGCTACGACTATCAGATAAAACTGAAACAACAGGAGGGCGCTAGACA
GATTGCGCGGAAAGAATGGAAAGAAATTGGTAAAATTAAAGAGATCAAAGAGG
GCTACCTGAGCTTAGTAATCCACGAGATCTCTAAAATGGTAATCAAATACAATG
CAATTATAGCGATGGAGGATTTGTCTTATGGTTTTAAAAAAGGGCGCTTTAAGG
TCGAACGGCAAGTTTACCAGAAATTTGAAACCATGCTCATCAATAAACTCAACT
ATCTGGTATTTAAAGATATTTCGATTACCGAGAATGGCGGTCTCCTGAAAGGTT
ATCAGCTGACATACATTCCTGATAAACTTAAAAACGTGGGTCATCAGTGCGGCT
GCATTTTTTATGTGCCTGCTGCATACACGAGCAAAATTGATCCGACCACCGGCTT
TGTGAATATCTTTAAATTTAAAGACCTGACAGTGGACGCAAAACGTGAATTCAT
TAAAAAATTTGACTCAATTCGTTATGACAGTGAAAAAAATCTGTTCTGCTTTACA
TTTGACTACAATAACTTTATTACGCAAAACACGGTCATGAGCAAATCATCGTGG
AGTGTGTATACATACGGCGTGCGCATCAAACGTCGCTTTGTGAACGGCCGCTTC
TCAAACGAAAGTGATACCATTGACATAACCAAAGATATGGAGAAAACGTTGGA
AATGACGGACATTAACTGGCGCGATGGCCACGATCTTCGTCAAGACATTATAGA
TTATGAAATTGTTCAGCACATATTCGAAATTTTCCGTTTAACAGTGCAAATGCGT
AACTCCTTGTCTGAACTGGAGGACCGTGATTACGATCGTCTCATTTCACCTGTAC
TGAACGAAAATAACATTTTTTATGACAGCGCGAAAGCGGGGGATGCACTTCCTA
AGGATGCCGATGCAAATGGTGCGTATTGTATTGCATTAAAAGGGTTATATGAAA
TTAAACAAATTACCGAAAATTGGAAAGAAGATGGTAAATTTTCGCGCGATAAAC
TCAAAATCAGCAATAAAGATTGGTTCGACTTTATCCAGAATAAGCGCTATCTCT
AA
CU-CH1: SEQ ID NO: 2:
atggatagtttgaaagatttcaccaatctgtaccctgtcagtaagacattgagatttgaattaaagcccgttggaaagactttagaaaatat
cgagaaagcaggtattttgaaagaggatgagcatcgtgcagaaagttatcggagggtgaagaaaataattgatacttatcataaggtatt
tatcgattcttctcttgaaaatatggctaaaatgggtattgagaatgaaataaaagcaatgctccaaagtttctgcgaattgtataaaaaaga
tcatcgcactgagggtgaagacaaggcattagataaaattcgagcagtacttcgtggcctgattgttggggattcactggtgtttgcgg
aagacgggaaaatacagtccaaaacgagaagtacgagagtttgttcaaagaaaagttgataaaagaaattttacctgattttgtgctctct
actgaggctgaaagcttgcattctctgttgaagaagctacgaggtcactgaaggagtttgatagctttacatcctactttgctggtttttac
gagaatagaaagaatatatactcgacgaaacctcaatccactgccattgcttatcgtcttattcatgagaacttgccgaagttcattgataa
tattcttgtttttcagaagatcaaagagcctatagccaaagagctggaacatattcgtgcggactttttctgccggggggtacataaaaaag
gatgagagattggaggatattttttcgttgaactattatatccacgtgttatctcaggctgggatcgaaaaatataacgcattgattgggaa
gattgtgacagaaggagatggagagatgaaagggctcaatgaacacatcaacctttacaaccaacaaagaggcagagaggatcggc
tccctctttttaggcct
CTTCACAAACAGATTCTATGCATTGCGGACACTAGCTATGAGGTCCCGTATAAAT
TTGAAAGTGACGAGGAAGTGTACCAATCAGTTAACGGCTTCCTTGATAACATTAG
CAGCAAACATATAGTCGAAAGATTACGCAAAATCGGCGATAACTATAACGGCTA
CAACCTGGATAAAATTTATATCGTGTCCAAATTTTACGAGAGCGTTAGCCAAAAA
ACCTACCGCGACTGGGAAACAATTAATACCGCCCTCGAAATTCATTACAATAATA
TCTTGCCGGGTAACGGTAAAAGTAAAGCCGACAAAGTAAAAAAAGCGGTTAAGA
ATGATTTACAGAAATCCATCACCGAAATAAATGAACTAGTGTCAAACTATAAGCT
GTGCAGTGACGACAACATCAAAGCGGAGACTTATATACATGAGATTAGCCATAT
CTTGAATAACTTTGAAGCACAGGAATTGAAATACAATCCGGAAATTCACCTAGTT
GAATCCGAGCTCAAAGCGAGTGAGCTTAAAAACGTGCTGGACGTGATCATGAAT
GCGTTTCATTGGTGTTCGGTTTTTATGACTGAGGAACTTGTTGATAAAGACAACA
ATTTTTATGCGGAACTGGAGGAGATTTACGATGAAATTTATCCAGTAATTAGTCT
GTACAACCTGGTTCGTAACTACGTTACCCAGAAACCGTACAGCACGAAAAAGAT
TAAATTGAACTTTGGAATACCGACGTTAGCAGACGGTTGGTCAAAGTCCAAAGA
GTATTCTAATAACGCTATCATACTGATGCGCGACAATCTGTATTATCTGGGCATC
TTTAATGCGAAGAATAAACCGGACAAGAAGATTATCGAGGGTAATACGTCAGAA
AATAAGGGTGACTACAAAAAGATGATTTATAATTTGCTCCCGGGTCCCAACAAA
ATGATCCCGAAAGTTTTCTTGAGCAGCAAGACGGGGGTGGAAACGTATAAACCG
AGCGCCTATATCCTAGAGGGGTATAAACAGAATAAACATATCAAGTCTTCAAAA
GACTTTGATATCACTTTCTGTCATGATCTGATCGACTACTTCAAAAACTGTATTGC
AATTCATCCCGAGTGGAAAAACTTCGGTTTTGATTTTAGCGACACCAGTACTTAT
GAAGACATTTCCGGGTTTTATCGTGAGGTAGAGTTACAAGGTTACAAGATTGATT
GGACATACATTAGCGAAAAAGACATTGATCTGCTGCAGGAAAAAGGTCAACTGT
ATCTGTTCCAGATATATAACAAAGATTTTTCGAAAAAATCAACCGGGAATGACA
ACCTTCACACCATGTACCTGAAAAATCTTTTCTCAGAAGAAAATCTTAAGGATAT
CGTCCTGAAACTTAACGGCGAAGCGGAAATCTTCTTCAGGAAGAGCAGCATAAA
GAACCCAATCATTCATAAAAAAGGCTCGATT
TTAGTCAACCGTACCTACGAAGCAGAAGAAAAAGACCAGTTTGGCAACATTCAA
ATTGTGCGTAAAAATATTCCGGAAAACATTTATCAGGAGCTGTACAAATACTTCA
ACGATAAAAGCGACAAAGAGCTGTCTGATGAAGCAGCCAAACTGAAGAATGTAG
TGGGACACCACGAGGCAGCGACGAATATAGTCAAGGACTATCGCTACACGTATG
ATAAATACTTCCTTCATATGCCTATTACGATCAATTTCAAAGCCAATAAAACGGG
TTTTATTAATGATAGGATCTTACAGTATATCGCTAAAGAAAAAGACTTACATGTG
ATCGGCATTGATCGGGGCGAGCGTAACCTGATCTACGTGTCCGTGATTGATACTT
GTGGTAATATAGTTGAACAGAAAAGCTTTAACATTGTAAACGGCTACGACTATCA
GATAAAACTGAAACAACAGGAGGGCGCTAGACAGATTGCGCGGAAAGAATGGA
AAGAAATTGGTAAAATTAAAGAGATCAAAGAGGGCTACCTGAGCTTAGTAATCC
ACGAGATCTCTAAAATGGTAATCAAATACAATGCAATTATAGCGATGGAGGATT
TGTCTTATGGTTTTAAAAAAGGGCGCTTTAAGGTCGAACGGCAAGTTTACCAGAA
ATTTGAAACCATGCTCATCAATAAACTCAACTATCTGGTATTTAAAGATATTTCG
ATTACCGAGAATGGCGGTCTCCTGAAAGGTTATCAGCTGACATACATTCCTGATA
AACTTAAAAACGTGGGTCATCAGTGCGGCTGCATTTTTTATGTGCCTGCTGCATA
CACGAGCAAAATTGATCCGACCACCGGCTTTGTGAATATCTTTAAATTTAAAGAC
CTGACAGTGGACGCAAAACGTGAATTCATTAAAAAATTTGACTCAATTCGTTATG
ACAGTGAAAAAAATCTGTTCTGCTTTACATTTGACTACAATAACTTTATTACGCA
AAACACGGTCATGAGCAAATCATCGTGGAGTGTGTATACATACGGCGTGCGCAT
CAAACGTCGCTTTGTGAACGGCCGCTTCTCAAACGAAAGTGATACCATTGACATA
ACCAAAGATATGGAGAAAACGTTGGAAATGACGGACATTAACTGGCGCGATGGC
CACGATCTTCGTCAAGACATTATAGATTATGAAATTGTTCAGCACATATTCGAAA
TTTTCCGTTTAACAGTGCAAATGCGTAACTCCTTGTCTGAACTGGAGGACCGTGA
TTACGATCGTCTCATTTCACCTGTACTGAACGAAAATAACATTTTTTATGACAGC
GCGAAAGCGGGGGATGCACTTCCTAAGGATGCCGATGCAAATGGTGCGTATTGT
ATTGCATTAAAAGGGTTATATGAAATTAAACAAATTACCGAAAATTGGAAAGAA
GATGGTAAATTTTCGCGCGATAAACTCAAAATCAGCAATAAAGATTGGTTCGACT
TTATCCAGAATAAGCGCTATCTCTAA
CU-CH5(M21): SEQ ID NO: 3:
atgactaaaacatttgattcagagttttttaatttgtactcgctgcaaaaaacggtacgctttgagttaaaacccgtgggagaaaccgcgtc
atttgtggaagactttaaaaacgagggcttgaaacgtgttgtgagcgaagatgaaaggcgagccgtcgattaccagaaagttaaggaa
ataattgacgattaccatcgggatttcattgaagaaagtttaaattattttccggaacaggtgagtaaagatgctcttgagcaggcgtttcat
ctttatcagaaactgaaggcagcaaaagttgaggaaagggaaaaagcgctgaaagaatgggaagcgctgcagaaaaagctacgtg
aaaaagtggtgaaatgcttctcggactcgaataaagcccgcttctcaaggattgataaaaaggaactgattaaggaagacctgataaatt
ggttggtcgcccagaatcgcgaggatgatatccctacggtcgaaacgtttaacaacttcaccacatattttaccggcttccatgagaatc
gtaaaaatatttactccaaagatgatcacgccaccgctattagctttcgccttattcatgaaaatcttccaaagatttgacaacgtgattag
cttcaataagttgaaagagggtttccctgaattaaaatttgataaagtgaaagaggatttagaagtagattatgatctgaagcatgcgtttg
aaatagaatatttcgttaacttcgtgacccaagcgggcatagatcagtataattatctgttaggagggaaaaccctggaggacgggacg
aaaaaacaagggatgaatgagcaaattaatctgttcaaacaacagcaaacgcgagataaagcgcgtcagattcccaaactgatcccc
CTTCACAAACAGATTCTATGCATTGCGGACACTAGCTATGAGGTCCCGTATAAAT
TTGAAAGTGACGAGGAAGTGTACCAATCAGTTAACGGCTTCCTTGATAACATTAG
CAGCAAACATATAGTCGAAAGATTACGCAAAATCGGCGATAACTATAACGGCTA
CAACCTGGATAAAATTTATATCGTGTCCAAATTTTACGAGAGCGTTAGCCAAAAA
ACCTACCGCGACTGGGAAACAATTAATACCGCCCTCGAAATTCATTACAATAATA
TCTTGCCGGGTAACGGTAAAAGTAAAGCCGACAAAGTAAAAAAAGCGGTTAAGA
ATGATTTACAGAAATCCATCACCGAAATAAATGAACTAGTGTCAAACTATAAGCT
GTGCAGTGACGACAACATCAAAGCGGAGACTTATATACATGAGATTAGCCATAT
CTTGAATAACTTTGAAGCACAGGAATTGAAATACAATCCGGAAATTCACCTAGTT
GAATCCGAGCTCAAAGCGAGTGAGCTTAAAAACGTGCTGGACGTGATCATGAAT
GCGTTTCATTGGTGTTCGGTTTTTATGACTGAGGAACTTGTTGATAAAGACAACA
ATTTTTATGCGGAACTGGAGGAGATTTACGATGAAATTTATCCAGTAATTAGTCT
GTACAACCTGGTTCGTAACTACGTTACCCAGAAACCGTACAGCACGAAAAAGAT
TAAATTGAACTTTGGAATACCGACGTTAGCAGACGGTTGGTCAAAGTCCAAAGA
GTATTCTAATAACGCTATCATACTGATGCGCGACAATCTGTATTATCTGGGCATC
TTTAATGCGAAGAATAAACCGGACAAGAAGATTATCGAGGGTAATACGTCAGAA
AATAAGGGTGACTACAAAAAGATGATTTATAATTTGCTCCCGGGTCCCAACAAA
ATGATCCCGAAAGTTTTCTTGAGCAGCAAGACGGGGGTGGAAACGTATAAACCG
AGCGCCTATATCCTAGAGGGGTATAAACAGAATAAACATATCAAGTCTTCAAAA
GACTTTGATATCACTTTCTGTCATGATCTGATCGACTACTTCAAAAACTGTATTGC
AATTCATCCCGAGTGGAAAAACTTCGGTTTTGATTTTAGCGACACCAGTACTTAT
GAAGACATTTCCGGGTTTTATCGTGAGGTAGAGTTACAAGGTTACAAGATTGATT
GGACATACATTAGCGAAAAAGACATTGATCTGCTGCAGGAAAAAGGTCAACTGT
ATCTGTTCCAGATATATAACAAAGATTTTTCGAAAAAATCAACCGGGAATGACA
ACCTTCACACCATGTACCTGAAAAATCTTTTCTCAGAAGAAAATCTTAAGGATAT
CGTCCTGAAACTTAACGGCGAAGCGGAAATCTTCTTCAGGAAGAGCAGCATAAA
GAACCCAATCATTCATAAAAAAGGCTCGATTTTAGTCAACCGTACCTACGAAGCA
GAAGAAAAAGACCAGTTTGGCAACATTCAAATTGTGCGTAAAAATATTCCGGAA
AACATTTATCAGGAGCTGTACAAATACTTCAACGATAAAAGCGACAAAGAGCTG
TCTGATGAAGCAGCCAAACTGAAGAATGTAGTGGGACACCACGAGGCAGCGACG
AATATAGTCAAGGACTATCGCTACACGTATGATAAATACTTCCTTCATATGCCTA
TTACGATCAATTTCAAAGCCAATAAAACGGGTTTTATTAATGATAGGATCTTACA
GTATATCGCTAAAGAAAAAGACTTACATGTGATCGGCATTGATCGGGGCGAGCG
TAACCTGATCTACGTGTCCGTGATTGATACTTGTGGTAATATAGTTGAACAGAAA
AGCTTTAACATTGTAAACGGCTACGACTATCAGATAAAACTGAAACAACAGGAG
GGCGCTAGACAGATTGCGCGGAAAGAATGGAAAGAAATTGGTAAAATTAAAGA
GATCAAAGAGGGCTACCTGAGCTTAGTAATCCACGAGATCTCTAAAATGGTAAT
CAAATACAATGCAATTATAGCGATGGAGGATTTGTCTTATGGTTTTAAAAAAGGG
CGCTTTAAGGTCGAACGGCAAGTTTACCAGAAATTTGAAACCATGCTCATCAATA
AACTCAACTATCTGGTATTTAAAGATATTTCGATTACCGAGAATGGCGGTCTCCT
GAAAGGTTATCAGCTGACATACATTCCTGATAAACTTAAAAACGTGGGTCATCAG
TGCGGCTGCATTTTTTATGTGCCTGCTGCATACACGAGCAAAATTGATCCGACCA
CCGGCTTTGTGAATATCTTTAAATTTAAAGACCTGACAGTGGACGCAAAACGTGA
ATTCATTAAAAAATTTGACTCAATTCGTTATGACAGTGAAAAAAATCTGTTCTGC
TTTACATTTGACTACAATAACTTTATTACGCAAAACACGGTCATGAGCAAATCAT
CGTGGAGTGTGTATACATACGGCGTGCGCATCAAACGTCGCTTTGTGAACGGCCG
CTTCTCAAACGAAAGTGATACCATTGACATAACCAAAGATATGGAGAAAACGTT
GGAAATGACGGACATTAACTGGCGCGATGGCCACGATCTTCGTCAAGACATTAT
AGATTATGAAATTGTTCAGCACATATTCGAAATTTTCCGTTTAACAGTGCAAATG
CGTAACTCCTTGTCTGAACTGGAGGACCGTGATTACGATCGTCTCATTTCACCTGT
ACTGAACGAAAATAACATTTTTTATGACAGCGCGAAAGCGGGGGATGCACTTCC
TAAGGATGCCGATGCAAATGGTGCGTATTGTATTGCATTAAAAGGGTTATATGAA
ATTAAACAAATTACCGAAAATTGGAAAGAAGATGGTAAATTTTCGCGCGATAAA
CTCAAAATCAGCAATAAAGATTGGTTCGACTTTATCCAGAATAAGCGCTATCTCT
AA
CU-CH4: SEQ ID NO: 4:
atgactaaaacatttgattcagagttttttaatttgtactcgctgcaaaaaacggtacgctttgagttaaaacccgtgggagaaaccgcgtc
atttgtggaagactttaaaaacgagggcttgaaacgtgttgtgagcgaagatgaaaggcgagccgtcgattaccagaaagttaaggaa
ataattgacgattaccatcgggatttcattgaagaaagtttaaattattttccggaacaggtgagtaaagatgctcttgagcaggcgtttcat
ctttatcagaaactgaaggcagcaaaagttgaggaaagggaaaaagcgctgaaagaatgggaagcgctgcagaaaaagctacgtg
aa
aaagtggtgaaatgcttctcggactcgaataaagcccgcttctcaaggattgataaaaaggaactgattaaggaagacctgataaattg
gttggtcgcccagaatcgcgaggatgatatccctacggtcgaaacgtttaacaacTTTGCGACTAGCTTTAAAGAT
TACTTCAAGAACCGTGCAAATTGCTTTTCAGCGGACGATATTTCATCAAGCAGCT
GCCATCGCATCGTCAACGACAATGCAGAGATATTCTTTTCAAATGCGCTGGTCTA
CCGCCGGATCGTAAAATCGCTGAGCAATGACGATATCAACAAAATTTCGGGCGA
TATGAAAGATTCATTAAAAGAAATGAGTCTGGAAGAAATATATTCTTACGAGAA
GTATGGGGAATTTATTACCCAGGAAGGCATTAGCTTCTATAATGATATCTGTGGG
AAAGTGAATTCTTTTATGAACCTGTATTGTCAGAAAAATAAAGAAAACAAAAAT
TTATACAAACTTCAGAAACTTCACAAACAGATTCTATGCATTGCGGACACTAGCT
ATGAGGTCCCGTATAAATTTGAAAGTGACGAGGAAGTGTACCAATCAGTTAACG
GCTTCCTTGATAACATTAGCAGCAAACATATAGTCGAAAGATTACGCAAAATCG
GCGATAACTATAACGGCTACAACCTGGATAAAATTTATATCGTGTCCAAATTTTA
CGAGAGCGTTAGCCAAAAAACCTACCGCGACTGGGAAACAATTAATACCGCCCT
CGAAATTCATTACAATAATATCTTGCCGGGTAACGGTAAAAGTAAAGCCGACAA
AGTAAAAAAAGCGGTTAAGAATGATTTACAGAAATCCATCACCGAAATAAATGA
ACTAGTGTCAAACTATAAGCTGTGCAGTGACGACAACATCAAAGCGGAGACTTA
TATACATGAGATTAGCCATATCTTGAATAACTTTGAAGCACAGGAATTGAAATAC
AATCCGGAAATTCACCTAGTTGAATCCGAGCTCAAAGCGAGTGAGCTTAAAAAC
GTGCTGGACGTGATCATGAATGCGTTTCATTGGTGTTCGGTTTTTATGACTGAGG
AACTTGTTGATAAAGACAACAATTTTTATGCGGAACTGGAGGAGATTTACGATGA
AATTTATCCAGTAATTAGTCTGTACAACCTGGTTCGTAACTACGTTACCCAGAAA
CCGTACAGCACGAAAAAGATTAAATTGAACTTTGGAATACCGACGTTAGCAGAC
GGTTGGTCAAAGTCCAAAGAGTATTCTAATAACGCTATCATACTGATGCGCGACA
ATCTGTATTATCTGGGCATCTTTAATGCGAAGAATAAACCGGACAAGAAGATTAT
CGAGGGTAATACGTCAGAAAATAAGGGTGACTACAAAAAGATGATTTATAATTT
GCTCCCGGGTCCCAACAAAATGATCCCGAAAGTTTTCTTGAGCAGCAAGACGGG
GGTGGAAACGTATAAACCGAGCGCCTATATCCTAGAGGGGTATAAACAGAATAA
ACATATCAAGTCTTCAAAAGACTTTGATATCACTTTCTGTCATGATCTGATCGACT
ACTTCAAAAACTGTATTGCAATTCATCCCGAGTGGAAAAACTTCGGTTTTGATTT
TAGCGACACCAGTACTTATGAAGACATTTCCGGGTTTTATCGTGAGGTAGAGTTA
CAAGGTTACAAGATTGATTGGACATACATTAGCGAAAAAGACATTGATCTGCTG
CAGGAAAAAGGTCAACTGTATCTGTTCCAGATATATAACAAAGATTTTTCGAAAA
AATCAACCGGGAATGACAACCTTCACACCATGTACCTGAAAAATCTTTTCTCAGA
AGAAAATCTTAAGGATATCGTCCTGAAACTTAACGGCGAAGCGGAAATCTTCTTC
AGGAAGAGCAGCATAAAGAACCCAATCATTCATAAAAAAGGCTCGATTTTAGTC
AACCGTACCTACGAAGCAGAAGAAAAAGACCAGTTTGGCAACATTCAAATTGTG
CGTAAAAATATTCCGGAAAACATTTATCAGGAGCTGTACAAATACTTCAACGATA
AAAGCGACAAAGAGCTGTCTGATGAAGCAGCCAAACTGAAGAATGTAGTGGGAC
ACCACGAGGCAGCGACGAATATAGTCAAGGACTATCGCTACACGTATGATAAAT
ACTTCCTTCATATGCCTATTACGATCAATTTCAAAGCCAATAAAACGGGTTTTATT
AATGATAGGATCTTACAGTATATCGCTAAAGAAAAAGACTTACATGTGATCGGC
ATTGATCGGGGCGAGCGTAACCTGATCTACGTGTCCGTGATTGATACTTGTGGTA
ATATAGTTGAACAGAAAAGCTTTAACATTGTAAACGGCTACGACTATCAGATAA
AACTGAAACAACAGGAGGGCGCTAGACAGATTGCGCGGAAAGAATGGAAAGAA
ATTGGTAAAATTAAAGAGATCAAAGAGGGCTACCTGAGCTTAGTAATCCACGAG
ATCTCTAAAATGGTAATCAAATACAATGCAATTATAGCGATGGAGGATTTGTCTT
ATGGTTTTAAAAAAGGGCGCTTTAAGGTCGAACGGCAAGTTTACCAGAAATTTG
AAACCATGCTCATCAATAAACTCAACTATCTGGTATTTAAAGATATTTCGATTAC
CGAGAATGGCGGTCTCCTGAAAGGTTATCAGCTGACATACATTCCTGATAAACTT
AAAAACGTGGGTCATCAGTGCGGCTGCATTTTTTATGTGCCTGCTGCATACACGA
GCAAAATTGATCCGACCACCGGCTTTGTGAATATCTTTAAATTTAAAGACCTGAC
AGTGGACGCAAAACGTGAATTCATTAAAAAATTTGACTCAATTCGTTATGACAGT
GAAAAAAATCTGTTCTGCTTTACATTTGACTACAATAACTTTATTACGCAAAACA
CGGTCATGAGCAAATCATCGTGGAGTGTGTATACATACGGCGTGCGCATCAAAC
GTCGCTTTGTGAACGGCCGCTTCTCAAAGAAAGTGATACCATTGACATAACCAA
AGATATGGAGAAAACGTTGGAAATGACGGACATTAACTGGCGCGATGGCCACGA
TCTTCGTCAAGACATTATAGATTATGAAATTGTTCAGCACATATTCGAAATTTTCC
GTTTAACAGTGCAAATGCGTAACTCCTTGTCTGAACTGGAGGACCGTGATTACGA
TCGTCTCATTTCACCTGTACTGAACGAAAATAACATTTTTTATGACAGCGCGAAA
GCGGGGGATGCACTTCCTAAGGATGCCGATGCAAATGGTGCGTATTGTATTGCAT
TAAAAGGGTTATATGAAATTAAACAAATTACCGAAAATTGGAAAGAAGATGGTA
AATTTTCGCGCGATAAACTCAAAATCAGCAATAAAGATTGGTTC GACTTTATCCA
GAATAAGCGCTATCTCTAA
CU-CH3: SEQ ID NO: 5: nucleic acid sequence
atgaccaataaattcactaaccagtattctctctctaagaccctgcgctttgaactgattccgcaggggaaaaccttggagttcattcaaga
aaaaggcctcttgtctcaggataaacagagggctgaatcttaccaagaaatgaagaaaactattgataagtttcataaatatttcattgattt
agccttgtctaacgccaaattaactcacttggaaacgtatctggagttatacaacaaatctgccgaaactaagaaagaacagaaatttaa
agacgatttgaaaaaagtacaggacaatctgcgtaaagaaattgtcaaatccttcagtgacggcgatgctaaaagcatttttgccattctg
gacaaaaaagagttgattactgtggaattagaaaagtggtttgaaaacaatgagcagaaagacatctacttcgatgagaaattcaaaact
ttcaccacctattttacaggatttcatcaaaaccggaagaacatgtactcagtagaaccgaactccacggccattgcgtatcgtttgatcc
atgagaatctgcctaaatttctggagaatgcgaaagcctttgaaaagattaagcaggicgaatcgctgcaagtgaattttcgtgaactcat
gggcgaattt
ggtgacgaaggictaatcttcgttaacgaactggaagaaatgtttcagattaattactacaatgacgtgctatcgcagaacggtatcacaa
tctacaatagtattatctcagggttcacaaaaaacgatataaaatacaaaggcctgaacgagtatatcaataactacaaccaaacaaagg
acaaaaaggataggcttccgaaactgaagcagCTTCACAAACAGATTCTATGCATTGCGGACACTA
GCTATGAGGTCCCGTATAAATTTGAAAGTGACGAGGAAGTGTACCAATCAGTTA
ACGGCTTCCTTGATAACATTAGCAGCAAACATATAGTCGAAAGATTACGCAAAA
TCGGCGATAACTATAACGGCTACAACCTGGATAAAATTTATATCGTGTCCAAATT
TTACGAGAGCGTTAGCCAAAAAACCTACCGCGACTGGGAAACAATTAATACCGC
CCTCGAAATTCATTACAATAATATCTTGCCGGGTAACGGTAAAAGTAAAGCCGAC
AAAGTAAAAAAAGCGGTTAAGAATGATTTACAGAAATCCATCACCGAAATAAAT
GAACTAGTGTCAAACTATAAGCTGTGCAGTGACGACAACATCAAAGCGGAGACT
TATATACATGAGATTAGCCATATCTTGAATAACTTTGAAGCACAGGAATTGAAAT
ACAATCCGGAAATTCACCTAGTTGAATCCGAGCTCAAAGCGAGTGAGCTTAAAA
ACGTGCTGGACGTGATCATGAATGCGTTTCATTGGTGTTCGGTTTTTATGACTGA
GGAACTTGTTGATAAAGACAACAATTTTTATGCGGAACTGGAGGAGATTTACGAT
GAAATTTATCCAGTAATTAGTCTGTACAACCTGGTTCGTAACTACGTTACCCAGA
AACCGTACAGCACGAAAAAGATTAAATTGAACTTTGGAATACCGACGTTAGCAG
ACGGTTGGTCAAAGTCCAAAGAGTATTCTAATAACGCTATCATACTGATGCGCGA
CAATCTGTATTATCTGGGCATCTTTAATGCGAAGAATAAACCGGACAAGAAGATT
ATCGAGGGTAATACGTCAGAAAATAAGGGTGACTACAAAAAGATGATTTATAAT
TTGCTCCCGGGTCCCAACAAAATGATCCCGAAAGTTTTCTTGAGCAGCAAGACGG
GGGTGGAAACGTATAAACCGAGCGCCTATATCCTAGAGGGGTATAAACAGAATA
AACATATCAAGTCTTCAAAAGACTTTGATATCACTTTCTGTCATGATCTGATCGA
CTACTTCAAAAACTGTATTGCAATTCATCCCGAGTGGAAAAACTTCGGTTTTGAT
TTTAGCGACACCAGTACTTATGAAGACATTTCCGGGTTTTATCGTGAGGTAGAGT
TACAAGGTTACAAGATTGATTGGACATACATTAGCGAAAAAGACATTGATCTGCT
GCAGGAAAAAGGTCAACTGTATCTGTTCCAGATATATAACAAAGATTTTTCGAAA
AAATCAACCGGGAATGACAACCTTCACACCATGTACCTGAAAAATCTTTTCTCAG
AAGAAAATCTTAAGGATATCGTCCTGAAACTTAACGGCGAAGCGGAAATCTTCTT
CAGGAAGAGCAGCATAAAGAACCCAATCATTCATAAAAAAGGCTCGATTTTAGT
CAACCGTACCTACGAAGCAGAAGAAAAAGACCAGTTTGGCAACATTCAAATTGT
GCGTAAAAATATTCCGGAAAACATTTATCAGGAGCTGTACAAATACTTCAACGAT
AAAAGCGACAAAGAGCTGTCTGATGAAGCAGCCAAACTGAAGAATGTAGTGGG
ACACCACGAGGCAGCGACGAATATAGTCAAGGACTATCGCTACACGTATGATAA
ATACTTCCTTCATATGCCTATTACGATCAATTTCAAAGCCAATAAAACGGGTTTT
ATTAATGATAGGATCTTACAGTATATCGCTAAAGAAAAAGACTTACATGTGATCG
GCATTGATCGGGGCGAGCGTAACCTGATCTACGTGTCCGTGATTGATACTTGTGG
TAATATAGTTGAACAGAAAAGCTTTAACATTGTAAACGGCTACGACTATCAGATA
AAACTGAAACAACAGGAGGGCGCTAGACAGATTGCGCGGAAAGAATGGAAAGA
AATTGGTAAAATTAAAGAGATCAAAGAGGGCTACCTGAGCTTAGTAATCCACGA
GATCTCTAAAATGGTAATCAAATACAATGCAATTATAGCGATGGAGGATTTGTCT
TATGGTTTTAAAAAAGGGCGCTTTAAGGTCGAACGGCAAGTTTACCAGAAATTTG
AAACCATGCTCATCAATAAACTCAACTATCTGGTATTTAAAGATATTTCGATTAC
CGAGAATGGCGGTCTCCTGAAAGGTTATCAGCTGACATACATTCCTGATAAACTT
AAAAACGTGGGTCATCAGTGCGGCTGCATTTTTTATGTGCCTGCTGCATACACGA
GCAAAATTGATCCGACCACCGGCTTTGTGAATATCTTTAAATTTAAAGACCTGAC
AGTGGACGCAAAACGTGAATTCATTAAAAAATTTGACTCAATTCGTTATGACAGT
GAAAAAAATCTGTTCTGCTTTACATTTGACTACAATAACTTTATTACGCAAAACA
CGGTCATGAGCAAATCATCGTGGAGTGTGTATACATACGGCGTGCGCATCAAAC
GTCGCTTTGTGAACGGCCGCTTCTCAAACGAAAGTGATACCATTGACATAACCAA
AGATATGGAGAAAACGTTGGAAATGACGGACATTAACTGGCGCGATGGCCACGA
TCTTCGTCAAGACATTATAGATTATGAAATTGTTCAGCACATATTCGAAATTTTCC
GTTTAACAGTGCAAATGCGTAACTCCTTGTCTGAACTGGAGGACCGTGATTACGA
TCGTCTCATTTCACCTGTACTGAACGAAAATAACATTTTTTATGACAGCGCGAAA
GCGGGGGATGCACTTCCTAAGGATGCCGATGCAAATGGTGCGTATTGTATTGCAT
TAAAAGGGTTATATGAAATTAAACAAATTACCGAAAATTGGAAAGAAGATGGTA
AATTTTCGCGCGATAAACTCAAAATCAGCAATAAAGATTGGTTCGACTTTATCCA
GAATAAGCGCTATCTCTAA
CU-CH6: SEQ ID NO: 6: nucleic acid sequence
atgactaaaacangattcagagttttttaatttgtactcgctgcaaaaaacggtacgctttgagttaaaacccgtgggagaaaccgcgtc
atttgtggaagactttaaaaacgagggcttgaaacgtgttgtgagcgaagatgaaaggcgagccgtcgattaccagaaagttaaggaa
ataattgacgattaccatcgggatttcattgaagaaagtttaaattattttccggaacaggtgagtaaagatgctcttgagcaggcgtttcat
ctttatcagaaactgaaggcagcaaaagttgaggaaagggaaaaagcgctgaaagaatgggaagcgctgcagaaaaagctacgtg
aaaaagtggtgaaatgcttctcggactcgaataaagcccgcttctcaaggattgataaaaaggaactgattaaggaagacctgataaatt
ggttggtcgcccagaatcgcgaggatgatatccctacggtcgaaacgtttaacaacttcaccacatattttaccggcttccatgagaatc
gtaaaaatatttactccaaagatgatcacgccaccgctattagctttcgccttattcatgaaaatcttccaaagttttttgacaacgtgattag
cttcaataagttgaaagagggtttccctgaattaaaatttgataaagtgaaagaggatttagaagtagattatgatctgaagcatgcgtttg
aaatagaatatttcgttaacttcgtgacccaagcgggcatagatcagtataattatctgttaggagggaaaaccctggaggacgggacg
aaaaaacaagggatgaatgagcaaattaatctgttcaaacaacagcaaacgcgagataaagcgcgtcagattcccaaactgatcccc
CTTCACAAACAGATTCTATGCATTGCGGACACTAGCTATGAGGTCCCGTATAAAT
TTGAAAGTGACGAGGAAGTGTACCAATCAGTTAACGGCTTCCTTGATAACATTAG
CAGCAAACATATAGTCGAAAGATTACGCAAAATCGGCGATAACTATAACGGCTA
CAACCTGGATAAAATTTATATCGTGTCCAAATTTTACGAGAGCGTTAGCCAAAAA
ACCTACCGCGACTGGGAAACAATTAATACCGCCCTCGAAATTCATTACAATAATA
TCTTGCCGGGTAACGGTAAAAGTAAAGCCGACAAAGTAAAAAAAGCGGTTAAGA
ATGATTTACAGAAATCCATCACCGAAATAAATGAACTAGTGTCAAACTATAAGCT
GTGCAGTGACGACAACATCAAAGCGGAGACTTATATACATGAGATTAGCCATAT
CTTGAATAACTTTGAAGCACAGGAATTGAAATACAATCCGGAAATTCACCTAGTT
GAATCCGAGCTCAAAGCGAGTGAGCTTAAAAACGTGCTGGACGTGATCATGAAT
GCGTTTCATTGGTGTTCGGTTTTTATGACTGAGGAACTTGTTGATAAAGACAACA
ATTTTTATGCGGAACTGGAGGAGATTTACGATGAAATTTATCCAGTAATTAGTCT
GTACAACCTGGTTCGTAACTACGTTACCCAGAAACCGTACAGCACGAAAAAGAT
TAAATTGAACTTTGGAATACCGACGTTAGCAGACGGTTGGTCAAAGTCCAAAGA
GTATTCTAATAACGCTATCATACTGATGCGCGACAATCTGTATTATCTGGGCATC
TTTAATGCGAAGAATAAACCGGACAAGAAGATTATCGAGGGTAATACGTCAGAA
AATAAGGGTGACTACAAAAAGATGATTTATAATTTGCTCCCGGGTCCCAACAAA
ATGATCCCGAAAGTTTTCTTGAGCAGCAAGACGGGGGTGGAAACGTATAAACCG
AGCGCCTATATCCTAGAGGGGTATAAACAGAATAAACATATCAAGTCTTCAAAA
GACTTTGATATCACTTTCTGTCATGATCTGATCGACTACTTCAAAAACTGTATTGC
AATTCATCCCGAGTGGAAAAACTTCGGTTTTGATTTTAGCGACACCAGTACTTAT
GAAGACATTTCCGGGTTTTATCGTGAGGTAGAGTTACAAGGTTACAAGATTGATT
GGACATACATTAGCGAAAAAGACATTGATCTGCTGCAGGAAAAAGGTCAACTGT
ATCTGTTCCAGATATATAACAAAGATTTTTCGAAAAAATCAACCGGGAATGACA
ACCTTCACACCATGTACCTGAAAAATCTTTTCTCAGAAGAAAATCTTAAGGATAT
CGTCCTGAAACTTAACGGCGAAGCGGAAATCTTCTTCAGGAAGAGCAGCATAAA
GAACCCAATCATTCATAAAAAAGGCTCGATTTTAGTCAACCGTACCTACGAAGCA
GAAGAAAAAGACCAGTTTGGCAACATTCAAATTGTGCGTAAAAATATTCCGGAA
AACATTTATCAGGAGCTGTACAAATACTTCAACGATAAAAGCGACAAAGAGCTG
TCTGATGAAGCAGCCAAACTGAAGAATGTAGTGGGACACCACGAGGCAGCGACG
AATATAGTCAAGGACTATCGCTACACGTATGATAAATACTTCCTTCATATGCCTA
TTACGATCAATTTCAAAGCCAATAAAACGGGTTTTATTAATGATAGGATCTTACA
GTATATCGCTAAAGAAAAAGACTTACATGTGATCGGCATTGATCGGGGCGAGCG
TAACCTGATCTACGTGTCCGTGATTGATACTTGTGGTAATATAGTTGAACAGAAA
AGCTTTAACATTGTAAACGGCTACGACTATCAGATAAAACTGAAACAACAGGAG
GGCGCTAGACAGATTGCGCGGAAAGAATGGAAAGAAATTGGTAAAATTAAAGA
GATCAAAGAGGGCTACCTGAGCTTAGTAATCCACGAGATCTCTAAAATGGTAAT
CAAATACAATGCAATTATAGCGATGGAGGATTTGTCTTATGGTTTTAAAAAAGGG
CGCTTTAAGGTCGAACGGCAAGTTTACCAGAAATTTGAAACCATGCTCATCAATA
AACTCAACTATCTGGTATTTAAAGATATTTCGATTACCGAGAATGGCGGTCTCCT
GAAAGGTTATCAGCTGACATACATTCCTGATAAACTTAAAAACGTGGGTCATCAG
TGCGGCTGCATTTTTTATGTGCCTGCTGCATACACGAGCaagattgatccgaccacgggcttcgc
caatgttctgaatctgtcgaaggtacgcaatgttgatgcgatcaaaagctttttttctaacttcaacgaaattagttatagcaagaaagaag
cccttttcaaattctcattcgatctggattcactgagtaagaaaggctttagtagctttgtgaaatttagtaagagtaaatggaacgtctacac
ctttggagaacgtatcataaagccaaagaataagcaaggttatcgggaggacaaaagaatcaacttgaccttcgagatgaagaagtta
cttaacgagtataaggtttcttagatcttgaaaataacttgattccgaatctcacgagtgccaacctgaaggatactttttggaaagagctat
tctttatcttcaagactacgctgcagctccgtaacagcgttactaacggtaaagaagatgtgctcatctctccggtcaaaaatgcgaagg
gtgaattcttcgtttcgggaacgcataacaagactcttccgcaagattgcgatgcgaacggtgcataccatattgcgttgaaaggtctgat
gatactcgaacgtaacaaccttgtacgtgaggagaaagatacgaaaaagattatggcgatttcaaacgtggattggttcgagtacgtgc
agaaacgtagaggcgttctgtaa
CU-CH7: SEQ ID NO: 7:
atgaacaactacgacgaattcaccaaactgtacccgatccagaaaaccatccgtttcgaactgaaaccgcagggtcgtaccatggaac
acctggaaaccttcaacttcttcgaagaagaccgtgaccgtgcggaaaaatacaaaatcctgaaagaagcgatcgacgaataccaca
aaaaattcatcgacgaacacctgaccaacatgtctctggactggaactctctgaaacagatctctgaaaaatactacaaatctcgtgaag
aaaaagacaaaaaagttttcctgtctgaacagaaacgtatgcgtcaggaaatcgtttctgaattcaaaaaagacgaccgtttcaaagacc
tgttctctaaaaaactgttctctgaactgctgaaagaagaaatctacaaaaaaggtaaccaccaggaaatcgacgcgctgaaatctttcg
acaaattctctggttacttcatcggtctgcacgaaaaccgtaaaaacatgtactctgacggtgacgaaatcaccgcgatctctaaccgtat
cgttaacgaaaacttcccgaaattcctggacaacctgcagaaataccaggaagcgcgtaaaaaatacccggaatggatcatcaaagc
ggaatctgcgctggttgcgcacaacatcaaaatggacgaagttttctctctggaatacttcaacaaagttctgaaccaggaaggtatcca
gcgttacaacctggcgctgggtggttacgttaccaaatctggtgaaaaaatgatgggtctgaacgacgcgctgaacctggcgcaccag
tctgaaaaatcttctaaaggtcgtatccacatgaccccgCTTCACAAACAGATTCTATGCATTGCGGACA
CTAGCTATGAGGTCCCGTATAAATTTGAAAGTGACGAGGAAGTGTACCAATCAG
TTAACGGCTTCCTTGATAACATTAGCAGCAAACATATAGTCGAAAGATTACGCAA
AATCGGCGATAACTATAACGGCTACAACCTGGATAAAATTTATATCGTGTCCAAA
TTTTACGAGAGCGTTAGCCAAAAAACCTACCGCGACTGGGAAACAATTAATACC
GCCCTCGAAATTCATTACAATAATATCTTGCCGGGTAACGGTAAAAGTAAAGCCG
ACAAAGTAAAAAAAGCGGTTAAGAATGATTTACAGAAATCCATCACCGAAATAA
ATGAACTAGTGTCAAACTATAAGCTGTGCAGTGACGACAACATCAAAGCGGAGA
CTTATATACATGAGATTAGCCATATCTTGAATAACTTTGAAGCACAGGAATTGAA
ATACAATCCGGAAATTCACCTAGTTGAATCCGAGCTCAAAGCGAGTGAGCTTAA
AAACGTGCTGGACGTGATCATGAATGCGTTTCATTGGTGTTCGGTTTTTATGACT
GAGGAACTTGTTGATAAAGACAACAATTTTTATGCGGAACTGGAGGAGATTTAC
GATGAAATTTATCCAGTAATTAGTCTGTACAACCTGGTTCGTAACTACGTTACCC
AGAAACCGTACAGCACGAAAAAGATTAAATTGAACTTTGGAATACCGACGTTAG
CAGACGGTTGGTCAAAGTCCAAAGAGTATTCTAATAACGCTATCATACTGATGCG
CGACAATCTGTATTATCTGGGCATCTTTAATGCGAAGAATAAACCGGACAAGAA
GATTATCGAGGGTAATACGTCAGAAAATAAGGGTGACTACAAAAAGATGATTTA
TAATTTGCTCCCGGGTCCCAACAAAATGATCCCGAAAGTTTTCTTGAGCAGCAAG
ACGGGGGTGGAAACGTATAAACCGAGCGCCTATATCCTAGAGGGGTATAAACAG
AATAAACATATCAAGTCTTCAAAAGACTTTGATATCACTTTCTGTCATGATCTGA
TCGACTACTTCAAAAACTGTATTGCAATTCATCCCGAGTGGAAAAACTTCGGTTT
TGATTTTAGCGACACCAGTACTTATGAAGACATTTCCGGGTTTTATCGTGAGGTA
GAGTTACAAGGTTACAAGATTGATTGGACATACATTAGCGAAAAAGACATTGAT
CTGCTGCAGGAAAAAGGTCAACTGTATCTGTTCCAGATATATAACAAAGATTTTT
CGAAAAAATCAACCGGGAATGACAACCTTCACACCATGTACCTGAAAAATCTTTT
CTCAGAAGAAAATCTTAAGGATATCGTCCTGAAACTTAACGGCGAAGCGGAAAT
CTTCTTCAGGAAGAGCAGCATAAAGAACCCAATCATTCATAAAAAAGGCTCGAT
TTTAGTCAACCGTACCTACGAAGCAGAAGAAAAAGACCAGTTTGGCAACATTCA
AATTGTGCGTAAAAATATTCCGGAAAACATTTATCAGGAGCTGTACAAATACTTC
AACGATAAAAGCGACAAAGAGCTGTCTGATGAAGCAGCCAAACTGAAGAATGTA
GTGGGACACCACGAGGCAGCGACGAATATAGTCAAGGACTATCGCTACACGTAT
GATAAATACTTCCTTCATATGCCTATTACGATCAATTTCAAAGCCAATAAAACGG
GTTTTATTAATGATAGGATCTTACAGTATATCGCTAAAGAAAAAGACTTACATGT
GATCGGCATTGATCGGGGCGAGCGTAACCTGATCTACGTGTCCGTGATTGATACT
TGTGGTAATATAGTTGAACAGAAAAGCTTTAACATTGTAAACGGCTACGACTATC
AGATAAAACTGAAACAACAGGAGGGCGCTAGACAGATTGCGCGGAAAGAATGG
AAAGAAATTGGTAAAATTAAAGAGATCAAAGAGGGCTACCTGAGCTTAGTAATC
CACGAGATCTCTAAAATGGTAATCAAATACAATGCAATTATAGCGATGGAGGAT
TTGTCTTATGGTTTTAAAAAAGGGCGCTTTAAGGTCGAACGGCAAGTTTACCAGA
AATTTGAAACCATGCTCATCAATAAACTCAACTATCTGGTATTTAAAGATATTTC
GATTACCGAGAATGGCGGTCTCCTGAAAGGTTATCAGCTGACATACATTCCTGAT
AAACTTAAAAACGTGGGTCATCAGTGCGGCTGCATTTTTTATGTGCCTGCTGCAT
ACACGAGCAAAATTGATCCGACCACCGGCTTTGTGAATATCTTTAAATTTAAAGA
CCTGACAGTGGACGCAAAACGTGAATTCATTAAAAAATTTGACTCAATTCGTTAT
GACAGTGAAAAAAATCTGTTCTGCTTTACATTTGACTACAATAACTTTATTACGC
AAAACACGGTCATGAGCAAATCATCGTGGAGTGTGTATACATACGGCGTGCGCA
TCAAACGTCGCTTTGTGAACGGCCGCTTCTCAAACGAAAGTGATACCATTGACAT
AACCAAAGATATGGAGAAAACGTTGGAAATGACGGACATTAACTGGCGCGATGG
CCACGATCTTCGTCAAGACATTATAGATTATGAAATTGTTCAGCACATATTCGAA
ATTTTCCGTTTAACAGTGCAAATGCGTAACTCCTTGTCTGAACTGGAGGACCGTG
ATTACGATCGTCTCATTTCACCTGTACTGAACGAAAATAACATTTTTTATGACAG
CGCGAAAGCGGGGGATGCACTTCCTAAGGATGCCGATGCAAATGGTGCGTATTG
TATTGCATTAAAAGGGTTATATGAAATTAAACAAATTACCGAAAATTGGAAAGA
AGATGGTAAATTTTCGCGCGATAAACTCAAAATCAGCAATAAAGATTGGTTCGA
CTTTATCCAGAATAAGCGCTATCTCTAA
CU-CH8: SEQ ID NO: 8:
atgcatacaggcggtcttcttagtatggacgcgaaagagttcacaggtcagtatccgttgtcgaaaacattacgattcgaacttcggccc
atcggccgcacgtgggataacctggaggcctcaggctacttagcggaagaccgccatcgtgccgaatgttatcctcgtgcgaaagag
ttattggatgacaaccatcgtgccttcctgaatcgtgtgttgccacaaatcgatatggattggcacccgattgcggaggccttttgtaaggt
acataaaaaccctggtaataaagaacttgcccaggattacaaccttcagttgtcaaagcgccgtaaggagatcagcgcatatcttcagg

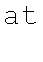

gcagatggctataaaggcctgttcgcgaagcccgccttagacgaagctatgaaaattgcgaaagaaaacgggaacgaaagtgatatt
gaggttctcgaagcgtttaacggttttagcgtatacttcaccggttatcatgagtcacgcgagaacatttatagcgatgaggatatggtga
gcgtagcctaccgaattactgaggataatttcccgcgctttgtctcaaacgctagatctttgataaattaaacgaaagccatccggatatta
tctctgaagtatcgggcaatcttggagttgatgacattggtaagtactttgacgtgtcgaactataacaattttctttcccaggccggtatag
atgactacaatcacattattggcggccatacaaccgaagacggactgatacaagcgtttaatgtcgtattgaacttacgtcaccaaaaag
accctggctttgaaaaaattcagttcaaacagCTTCACAAACAGATTCTATGCATTGCGGACACTAGC
TATGAGGTCCCGTATAAATTTGAAAGTGACGAGGAAGTGTACCAATCAGTTAAC
GGCTTCCTTGATAACATTAGCAGCAAACATATAGTCGAAAGATTACGCAAAATC
GGCGATAACTATAACGGCTACAACCTGGATAAAATTTATATCGTGTCCAAATTTT
ACGAGAGCGTTAGCCAAAAAACCTACCGCGACTGGGAAACAATTAATACCGCCC
TCGAAATTCATTACAATAATATCTTGCCGGGTAACGGTAAAAGTAAAGCCGACA
AAGTAAAAAAAGCGGTTAAGAATGATTTACAGAAATCCATCACCGAAATAAATG
AACTAGTGTCAAACTATAAGCTGTGCAGTGACGACAACATCAAAGCGGAGACTT
ATATACATGAGATTAGCCATATCTTGAATAACTTTGAAGCACAGGAATTGAAATA
CAATCCGGAAATTCACCTAGTTGAATCCGAGCTCAAAGCGAGTGAGCTTAAAAA
CGTGCTGGACGTGATCATGAATGCGTTTCATTGGTGTTCGGTTTTTATGACTGAG
GAACTTGTTGATAAAGACAACAATTTTTATGCGGAACTGGAGGAGATTTACGATG
AAATTTATCCAGTAATTAGTCTGTACAACCTGGTTCGTAACTACGTTACCCAGAA
ACCGTACAGCACGAAAAAGATTAAATTGAACTTTGGAATACCGACGTTAGCAGA
CGGTTGGTCAAAGTCCAAAGAGTATTCTAATAACGCTATCATACTGATGCGCGAC
AATCTGTATTATCTGGGCATCTTTAATGCGAAGAATAAACCGGACAAGAAGATTA
TCGAGGGTAATACGTCAGAAAATAAGGGTGACTACAAAAAGATGATTTATAATT
TGCTCCCGGGTCCCAACAAAATGATCCCGAAAGTTTTCTTGAGCAGCAAGACGG
GGGTGGAAACGTATAAACCGAGCGCCTATATCCTAGAGGGGTATAAACAGAATA
AACATATCAAGTCTTCAAAAGACTTTGATATCACTTTCTGTCATGATCTGATCGA
CTACTTCAAAAACTGTATTGCAATTCATCCCGAGTGGAAAAACTTCGGTTTTGAT
TTTAGCGACACCAGTACTTATGAAGACATTTCCGGGTTTTATCGTGAGGTAGAGT
TACAAGGTTACAAGATTGATTGGACATACATTAGCGAAAAAGACATTGATCTGCT
GCAGGAAAAAGGTCAACTGTATCTGTTCCAGATATATAACAAAGATTTTTCGAAA
AAATCAACCGGGAATGACAACCTTCACACCATGTACCTGAAAAATCTTTTCTCAG
AAGAAAATCTTAAGGATATCGTCCTGAAACTTAACGGCGAAGCGGAAATCTTCTT
CAGGAAGAGCAGCATAAAGAACCCAATCATTCATAAAAAAGGCTCGATTTTAGT
CAACCGTACCTACGAAGCAGAAGAAAAAGACCAGTTTGGCAACATTCAAATTGT
GCGTAAAAATATTCCGGAAAACATTTATCAGGAGCTGTACAAATACTTCAACGAT
AAAAGCGACAAAGAGCTGTCTGATGAAGCAGCCAAACTGAAGAATGTAGTGGG
ACACCACGAGGCAGCGACGAATATAGTCAAGGACTATCGCTACACGTATGATAA
ATACTTCCTTCATATGCCTATTACGATCAATTTCAAAGCCAATAAAACGGGTTTT
ATTAATGATAGGATCTTACAGTATATCGCTAAAGAAAAAGACTTACATGTGATCG
GCATTGATCGGGGCGAGCGTAACCTGATCTACGTGTCCGTGATTGATACTTGTGG
TAATATAGTTGAACAGAAAAGCTTTAACATTGTAAACGGCTACGACTATCAGATA
AAACTGAAACAACAGGAGGGCGCTAGACAGATTGCGCGGAAAGAATGGAAAGA
AATTGGTAAAATTAAAGAGATCAAAGAGGGCTACCTGAGCTTAGTAATCCACGA
GATCTCTAAAATGGTAATCAAATACAATGCAATTATAGCGATGGAGGATTTGTCT
TATGGTTTTAAAAAAGGGCGCTTTAAGGTCGAACGGCAAGTTTACCAGAAATTTG
AAACCATGCTCATCAATAAACTCAACTATCTGGTATTTAAAGATATTTCGATTAC
CGAGAATGGCGGTCTCCTGAAAGGTTATCAGCTGACATACATTCCTGATAAACTT
AAAAACGTGGGTCATCAGTGCGGCTGCATTTTTTATGTGCCTGCTGCATACACGA
GCAAAATTGATCCGACCACCGGCTTTGTGAATATCTTTAAATTTAAAGACCTGAC
AGTGGACGCAAAACGTGAATTCATTAAAAAATTTGACTCAATTCGTTATGACAGT
GAAAAAAATCTGTTCTGCTTTACATTTGACTACAATAACTTTATTACGCAAAACA
CGGTCATGAGCAAATCATCGTGGAGTGTGTATACATACGGCGTGCGCATCAAAC
GTCGCTTTGTGAACGGCCGCTTCTCAAACGAAAGTGATACCATTGACATAACCAA
AGATATGGAGAAAACGTTGGAAATGACGGACATTAACTGGCGCGATGGCCACGA
TCTTCGTCAAGACATTATAGATTATGAAATTGTTCAGCACATATTCGAAATTTTCC
GTTTAACAGTGCAAATGCGTAACTCCTTGTCTGAACTGGAGGACCGTGATTACGA
TCGTCTCATTTCACCTGTACTGAACGAAAATAACATTTTTTATGACAGCGCGAAA
GCGGGGGATGCACTTCCTAAGGATGCCGATGCAAATGGTGC GTATTGTATTGCAT
TAAAAGGGTTATATGAAATTAAACAAATTACCGAAAATTGGAAAGAAGATGGTA
AATTTTCGCGCGATAAACTCAAAATCAGCAATAAAGATTGGTTCGACTTTATCCA
GAATAAGCGCTATCTCTAA
CU-CH9: SEQ ID NO: 9 (M44):
atgactaaaacatttgattcagagatataatttgtactcgctgcaaaaaacggtacgctttgagttaaaacccgtgggagaaaccgcgtc
atttgtggaagactttaaaaacgagggcttgaaacgtgttgtgagcgaagatgaaaggcgagccgtcgattaccagaaagttaaggaa
ataattgacgattaccatcgggatttcattgaagaaagtttaaattattttccggaacaggtgagtaaagatgctcttgagcaggcgtttcat
ctttatcagaaactgaaggcagcaaaagttgaggaaagggaaaaagcgctgaaagaatgggaagcgctgcagaaaaagctacgtg
aaaaagtggtgaaatgcttctcggactcgaataaagcccgcttctcaaggattgataaaaaggaactgattaaggaagacctgataaatt
ggttggtcgcccagaatcgcgaggatgatatccctacggtcgaaacgtttaacaacttcaccacatattttaccggcttccatgagaatc
gtaaaaatatttactccaaagatgatcacgccaccgctattagctttcgccttattcatgaaaatcttccaaagttttttgacaacgtgattag
cttcaataagttgaaagagggtttccctgaattaaaatttgataaagtgaaagaggatttagaagtagattatgatctgaagcatgcgtttg
aaatagaatatttcgttaacttcgtgacccaagcgggcatagatcagtataattatctgttaggagggaaaaccctggaggacgggacg
aaaaaacaagggatgaatgagcaaattaatctgttcaaacaacagcaaacgcgagataaagcgcgtcagattcccaaactgatcccc
CTTCACAAACAGATTCTATGCATTGCGGACACTAGCTATGAGGTCCCGTATAAAT
TTGAAAGTGACGAGGAAGTGTACCAATCAGTTAACGGCTTCCTTGATAACATTAG
CAGCAAACATATAGTCGAAAGATTACGCAAAATCGGCGATAACTATAACGGCTA
CAACCTGGATAAAATTTATATCGTGTCCAAATTTTACGAGAGCGTTAGCCAAAAA
ACCTACCGCGACTGGGAAACAATTAATACCGCCCTCGAAATTCATTACAATAATA
TCTTGCCGGGTAACGGTAAAAGTAAAGCCGACAAAGTAAAAAAAGCGGTTAAGA
ATGATTTACAGAAATCCATCACCGAAATAAATGAACTAGTGTCAAACTATAAGCT
GTGCAGTGACGACAACATCAAAGCGGAGACTTATATACATGAGATTAGCCATAT
CTTGAATAACTTTGAAGCACAGGAATTGAAATACAATCCGGAAATTCACCTAGTT
GAATCCGAGCTCAAAGCGAGTGAGCTTAAAAACGTGCTGGACGTGATCATGAAT
GCGTTTCATTGGTGTTCGGTTTTTATGACTGAGGAACTTGTTGATAAAGACAACA
ATTTTTATGCGGAACTGGAGGAGATTTACGATGAAATTTATCCAGTAATTAGTCT
GTACAACCTGGTTCGTAACTACGTTACCCAGAAACCGTACAGCACGAAAAAGAT
TAAATTGAACTTTGGAATACCGACGTTAGCAGACGGTTGGTCAAAGTCCAAAGA
GTATTCTAATAACGCTATCATACTGATGCGCGACAATCTGTATTATCTGGGCATC
TTTAATGCGAAGAATAAACCGGACAAGAAGATTATCGAGGGTAATACGTCAGAA
AATAAGGGTGACTACAAAAAGATGATTTATAATTTGCTCCCGGGTCCCAACAAA
ATGATCCCGAAAGTTTTCTTGAGCAGCAAGACGGGGGTGGAAACGTATAAACCG
AGCGCCTATATCCTAGAGGGGTATAAACAGAATAAACATATCAAGTCTTCAAAA
GACTTTGATATCACTTTCTGTCATGATCTGATCGACTACTTCAAAAACTGTATTGC
AATTCATCCCGAGTGGAAAAACTTCGGTTTTGATTTTAGCGACACCAGTACTTAT
GAAGACATTTCCGGGTTTTATCGTGAGGTAGAGTTACAAGGTTACAAGATTGATT
GGACATACATTAGCGAAAAAGACATTGATCTGCTGCAGGAAAAAGGTCAACTGT
ATCTGTTCCAGATATATAACAAAGATTTTTCGAAAAAATCAACCGGGAATGACA
ACCTTCACACCATGTACCTGAAAAATCTTTTCTCAGAAGAAAATCTTAAGGATAT
CGTCCTGAAACTTAACGGCGAAGCGGAAATCTTCTTCAGGAAGAGCAGCATAAA
GAACCCAATCATTCATAAAAAAGGCTCGATTTTAGTCAACCGTACCTACGAAGCA
GAAGAAAAAGACCAGTTTGGCAACATTCAAATTGTGCGTAAAAATATTCCGGAA
AACATTTATCAGGAGCTGTACAAATACTTCAACGATAAAAGCGACAAAGAGCTG
TCTGATGAAGCAGCCAAACTGAAGAATGTAGTGGGACACCACGAGGCAGCGACG
AATATAGTCAAGGACTATCGCTACACGTATGATAAATACTTCCTTCATATGCCTA
TTACGATCAATTTCAAAGCCAATAAAACGGGTTTTATTAATGATAGGATCTTACA
GTATATCGCTAAAGAAAAAGACTTACATGTGATCGGCATTGATCGGGGCGAGCG
TAACCTGATCTACGTGTCCGTGATTGATACTTGTGGTAATATAGTTGAACAGAAA
AGCTTTAACATTGTAAACGGCTACGACTATCAGATAAAACTGAAACAACAGGAG
GGCGCTAGACAGATTGCGCGGAAAGAATGGAAAGAAATTGGTAAAATTAAAGA
GATCAAAGAGGGCTACCTGAGCTTAGTAATCCACGAGATCTCTAAAATGGTAAT
CAAATACAATGCAATTATAGCGATGGAGGATTTGTCTTATGGTTTTAAAAAAGGG
CGCTTTAAGGTCGAACGGCAAGTTTACCAGAAATTTGAAACCATGCTCATCAATA
AACTCAACTATCTGGTATTTAAAGATATTTCGATTACCGAGAATGGCGGTCTCCT
GAAAGGTTATCAGCTGACATACATTCCTGATAAACTTAAAAACGTGGGTCATCAG
TGCGGCTGCATTTTTTATGTGCCTGCTGCATACACGAGCAAAATTGATCCGACCA
CCGGCTTTGTGAATATCTTTAAATTTAAAGACCTGACAGTGGACGCAAAACGTGA
ATTCATTAAAAAATTTGACTCAATTCGTTATGACAGTGAAAAAAATCTGTTCTGC
TTTACATTTGACTACAATAACTTTATTACGCAAAACACGGTCATGAGCAAATCAT
CGTGGAGTGTGTATACATACGGCGTGCGCATCAAACGTCGCTTTGTGAACGGCCG
CTTCTCAAACGAAAGTGATACCATTGACATAACCAAAGATATGGAGAAAACGTT
GGAAATGACGGACATTAACTGGCGCGATGGCCACGATCTTCGTCAAGACATTAT
AGATTATGAAATTGTTCAGCACATATTCGAAATTTTCCGTTTAACAGTGCAAATG
CGTAACTCCTTGTCTGAACTGGAGGACCGTGATTACGATCGTCTCATTTCACCTGT
ACTGAACGAAAATAACATTTTTTATGACAGCGCGAAAGCGGGGGATGCACTTCC
TAAGGATGCCGATGCAAATGGTGCGTATTGTATTGCATTAAAAGGGTTATATGAA
ATTAAACAAATTACCGAAAATTGGAAAGAAGATGGTAAATTTTCGCGCGATAAA
CTCAAAATCAGCAATAAAGATTGGTTCGACTTTATCCAGAATAAGCGCTATCTCT
AA

In accordance with these embodiments, engineered chimeric nucleic acid guided nucleases of use here and described herein can be at least 80%, at least 85%, at least 90%, at least 95%, at least 99% or more identical to the following referenced nucleic acid or corresponding polypeptide sequences where constructs disclosed and claimed herein include, but are not limited to, CU_CH1: 1 to 927 bp from PC CAS12A, 928 to 3876 bp from a positive control derived from a Cas12a of Eubacterium rectale; CU_CH2: 1 to 912 bp from SC_CAS12A, 913 to 3861 bp from a positive control derived from a Cas12a of Eubacterium rectale; CU_CH3: 1 to 861 bp from FB_CAS12A, 862 to 3810 bp from a positive control derived from a Cas12a of Eubacterium rectal; CU_CH4:1 to 504 bp from TX_CAS12A, 505 to 3819 bp from a positive control derived from a Cas12a of Eubacterium rectale; CU_CH5: 1 to 900 bp from TX_CAS12A with mutation G218A, 901 to 3849 bp from a positive control derived from a Cas12a of Eubacterium rectale; CU_CH6: 1 to 900 bp from TX_CAS12A, 901 to 3174 bp from a positive control derived from a Cas12a of Eubacterium rectale; CU_CH7: 1 to 840 bp from, 841 to 3789 bp from a positive control derived from a Cas12a of Eubacterium rectale; CU_CH8 (M43): 1 to 846 bp from a Cas12a, 847 to 3795 bp from a positive control derived from a Cas12a of Eubacterium rectale; and CU_CH9: 1 to 900 bp from TX_CAS12A, 901 to 3849 bp from a positive control derived from a Cas12a of Eubacterium rectale and combinations thereof.

In other embodiments, engineered chimeric nucleic acid guided nucleases disclosed herein and of use here can be at least 80%, at least 85%, at least 90%, at least 95%, at least 99% or more identical to the following referenced nucleic acid sequences represented by SEQ ID NOs: 1 to 9 or corresponding polypeptides thereof.

In certain embodiments, engineered chimeric nucleic acid guided nucleases disclosed herein have been created for increased efficiency and accuracy of targeted gene editing in a subject. In accordance with these embodiments, these engineered chimeric nucleic acid guided nuclease constructs can be used at a commercially relevant level for targeted editing. In some embodiments, the engineered chimeric nucleic acid guided nucleases constructs disclosed herein have altered PAM recognition sequence for altered and improved editing capabilities such as on/off rates.

In certain embodiments, engineered chimeric nucleic acid guided nuclease construct represented by SEQ ID NO: 1 to 9, have been invented that enable altered and/or improved CRISPR-CAS12-like editing. In certain embodiments, the activity of these endonucleases has been measured in bacteria (e.g. E. coli), yeast and in human cells. In accordance with these embodiments, these gene editing systems can be used in multiple species including humans and other mammals. In certain embodiments, engineered chimeric nucleic acid guided nucleases of the instant invention can be used for targeted editing of a mammalian genome in order to target different genes having a recognized PAM sequence for improved editing and more directed targeting to improve accuracy and/or efficiency of genome editing. Several sequences have been identified to enable editing at commercially relevant levels. All sequences of the instantly claimed constructs combine sequences of at least two or more different starting Cas12a nucleases or Cas12a-like nucleases. In certain embodiments, the chimeric constructs of the instantly claimed invention have altered PAM recognition sequences for targeted gene editing.

Examples of target polynucleotides for use of engineered chimeric nucleic acid guided nucleases disclosed herein can include a sequence/gene or gene segment associated with a signaling biochemical pathway, e.g., a signaling biochemical pathway-associated gene or polynucleotide. Other embodiments contemplated herein concern examples of target polynucleotides related to a disease-associated gene or polynucleotide.

A “disease-associated” gene or polynucleotide can refer to any gene or polynucleotide which results in a transcription or translation product at an abnormal level compared to a control or results in an abnormal form in cells derived from disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level

It is understood by one of skill in the relevant art that examples of disease-associated genes and polynucleotides are available from. McKusick-Nathans Institute of Genetic Medicine, Johns Hopkins University (Baltimore, Md.) and National Center for Biotechnology Information, National Library of Medicine (Bethesda, Md.), available on the World Wide Web.

Genetic Disorders contemplated herein can include, but are not limited to,

Neoplasia: Genes linked to this disorder: PTEN; ATM; ATR; EGFR; ERBB2; ERBB3; ERBB4; Notch1; Notch2; Notch3; Notch4; AKT; AKT2; AKT3; HIF; HIFI a; HIF3a; Met; HRG; Bc12; PPAR alpha; PPAR gamma; WT1 (Wilms Tumor); FGF Receptor Family members (5 members: 1, 2, 3, 4, 5); CDKN2a; APC; RB (retinoblastoma); MEN1; VHL; BRCA1; BRCA2; AR (Androgen Receptor); TSG101; IGF; IGF Receptor; Igf1 (4 variants); Igf2 (3 variants); Igf 1 Receptor; Igf 2 Receptor; Bax; Bc12; caspases family (9 members: 1, 2, 3, 4, 6, 7, 8, 9, 12); Kras; Apc

Age-related Macular Degeneration: Genes linked to these disorders Abcr; Cc12; Cc2; cp (cemloplasmin); Timp3; cathepsinD; VIdlr; Ccr2

Schizophrenia Disorders: Genes linked to this disorder: Neuregulin1 (Nrg1); Erb4 (receptor for Neuregulin); Complexin1 (Cplx1); Tph1 Tryptophan hydroxylase; Tph2 Tryptophan hydroxylase 2; Neurexin 1; GSK3; GSK3a; GSK3b

Trinucleotide Repeat Disorders: Genes linked to this disorder: 5 HTT (Huntington's Dx); SBMA/SMAX1/AR (Kennedy's Dx); FXN/X25 (Friedrich's Ataxia); ATX3 (Machado-Joseph's Dx); ATXN1 and ATXN2 (spinocerebellar ataxias); DMPK (myotonic dystrophy); Atrophin-1 and Atn1 (DRPLA Dx); CBP (Creb-BP—global instability); VLDLR (Alzheimer's); Atxn7; Atxn10

Fragile X Syndrome: Genes linked to this disorder: FMR2; FXR1; FXR2; mGLURS

Secretase Related Disorders: Genes linked to this disorder: APH-1 (alpha and beta); Presenil n (Psenl); nicastrin (Ncstn); PEN-2

Others: Genes linked to this disorder: Nos1; Paip1; Nati; Nat2

Prion—related disorders: Gene linked to this disorder: Prp

ALS: Genes linked to this disorder: SOD1; ALS2; STEX; FUS; TARDBP; VEGF (VEGF-a; VEGF-b; VEGF-c)

Drug addiction: Genes linked to this disorder: Prkce (alcohol); Drd2; Drd4; ABAT (alcohol); GRIA2; GrmS; Grin1; Htr1b; Grin2a; Drd3; Pdyn; Gria1 (alcohol)

Autism: Genes linked to this disorder: Mecp2; BZRAP1; MDGA2; SemaSA; Neurexin 1; Fragile X (FMR2 (AFF2); FXR1; FXR2; MglurS)

Alzheimer's Disease Genes linked to this disorder: E1; CHIP; UCH; UBB; Tau; LRP; PICALM; Clusterin; PS1; SORL1; CR1; VIdlr; Uba1; Uba3; CHIP28 (Aqp1, Aquaporin 1); Uch11; Uch13; APP

Inflammation and Immune-related disorders Genes linked to this disorder: IL-10; IL-1 (IL-1a; IL-1b); IL-13; IL-17 (IL-17a (CTLA8); IL-17b; IL-17c; IL-17d; IL-17f); 11-23; Cx3cr1; ptpn22; TNFa; NOD2/CARD15 for IBD; IL-6; IL-12 (IL-12a; IL-12b); CTLA4; Cx3c11, AAT deficiency/mutations, AIDS (KIR3DL1, NKAT3, NKB1, ANIB11, KIR3DS1, IFNG, CXCL12, SDF1); Autoimmune lymphoproliferative syndrome (TNFRSF6, APT1, FAS, CD95, ALPS1A); Combined immunodeficiency, (IL2RG, SCIDX1, SCIDX, IMD4); HIV-1 (CCL5, SCYA5, D17S136E, TCP228), HIV susceptibility or infection (IL10, CSIF, CMKBR2, CCR2, CMKBR5, CCCKR5 (CCR5)); Immunodeficiencies (CD3E, CD3G, AICDA, AID, HIGM2, TNFRSF5, CD40, UNG, DGU, HIGM4, TNFSF5, CD4OLG, HIGM1, IGM, FOXP3, IPEX, AIID, XPID, PIDX, TNFRSF14B, TACI); Inflammation (IL-10, IL-1 (IL-1a, IL-1b), IL-13, IL-17 (IL-17a (CTLA8), IL-17b, IL-17c, IL-17d, IL-17f), 11-23, Cx3cr1, ptpn22, TNFa, NOD2/CARD15 for IBD, IL-6, IL-12 (IL-12a, IL-12b), CTLA4, Cx3c11); Severe combined immunodeficiencies (SCIDs)(JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, IMD4).

Parkinson's, Genes linked to this disorder: x-Synuclein; DJ-1; LRRK2; Parkin; PINK1

Blood and coagulation disorders: Genes linked to these disorders: Anemia (CDAN1, CDA1, RPS19, DBA, PKLR, PK1, NT5C3, UMPH I, PSN1, RHAG, RH50A, NRAMP2, SPTB, ALAS2, ANH I, ASB, ABCB7, ABC7, ASAT); Bare lymphocyte syndrome (TAPBP, TPSN, TAP2, ABCB3, PSF2, RINGI 1, MHC2TA, C2TA, RFX5, RFXAP, RFX5), Bleeding disorders (TBXA2R, P2RX I, P2X I); Factor H and factor H-like 1 (HF1, CFH, HUS); Factor V and factor VIII (MCFD2); Factor VII deficiency (F7); Factor X deficiency (F10); Factor XI deficiency (F11); Factor XII deficiency (F12, HAF); Factor XIIIA deficiency (F13A1, F13A); Factor XIIIB deficiency (F13B); Fanconi anemia (FANCA, FACA, FA1, FA, FAA, FAAP95, FAAP90, F1134064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, ICIAA1596); Hemophagocytic lymphohistiocytosis disorders (PRF1, HPLH2, UNC13D, MUNC13-4, HPLH3, HLH3, FHL3); Hemophilia A (F8, F8C, HEMA); Hemophilia B (F9, HEMB), Hemorrhagic disorders (PI, ATT, F5); Leukocyde deficiencies and disorders (ITGB2, CD18, LCAMB, LAD, EIF2B1, EIF2BA, EIF2B2, EIF2B3, EIF2B5, LVWM, CACH, CLE, EIF2B4); Sickle cell anemia (HBB); Thalassemia (HBA2, HBB, HBD, LCRB, HBA1).

Cell dysregulation and oncology disorders: Genes linked to these disorders: B-cell non-Hodgkin lymphoma (BCL7A, BCL7); Leukemia (TALI TCL5, SCL, TAL2, FLT3, NBS 1, NBS, ZNFNIAI, IK1, LYF1, HOXD4, HOX4B, BCR, CML, PHL, ALL, ARNT, KRAS2, RASK2, GMPS, AFIO, ARHGEFI2, LARG, KIAA0382, CALM, CLTH, CEBPA, CEBP, CHIC2, BTL, FLT3, KIT, PBT, LPP, NPM1, NUP214, D9S46E, CAN, CAIN, RUNX 1, CBFA2, AML1, WHSC 1 LI, NSD3, FLT3, AF1Q, NPM 1, NUMA1, ZNF145, PLZF, PML, MYL, STAT5B, AFI 0, CALM, CLTH, ARLI 1, ARLTS1, P2RX7, P2X7, BCR, CML, PHL, ALL, GRAF, NFI, VRNF, WSS, NFNS, PTPNI 1, PTP2C, SHP2, NS 1, BCL2, CCND1, PRAD1, BCL1, TCRA, GATA1, GF1, ERYF1, NFE1, ABL1, NQO1, DIA4, NMOR1, NUP2I4, D9S46E, CAN, CAIN).

Metabolic, liver, kidney disorders: Genes linked to these disorders: Amyloid neuropathy (TTR, PALS); Amyloidosis (APOA1, APP, AAA, CVAP, AD1, GSN, FGA, LYZ, UR, PALS); Cirrhosis (KATI 8, KRT8, CaHlA, NAIC, TEX292, KIAA1988); Cystic fibrosis (CFTR, ABCC7, CF, MRP7); Glycogen storage diseases (SLC2A2, GLUT2, G6PC, G6PT, G6PT1, GAA, LAMP2, LAMPS, AGL, GDE, GBE1, GYS2, PYGL, PFKM); Hepatic adenoma, 142330 (TCF1, HNF1A, MODY3), Hepatic failure, early onset, and neurologic disorder (SCOD1, SCO1), Hepatic lipase deficiency (LIPC), Hepatoblastoma, cancer and carcinomas (CTNNB1, PDGFRL, PDGRL, PRLTS, AXIN1, AXIN, CTNNB1, TP53, P53, LFS1, IGF2R, MPRI, MET, CASP8, MCHS; Medullary cystic kidney disease (UMOD, HNFJ, FJHN, MCKD2, ADMCKD2); Phenylketonuria (PAH, PKU1, QDPR, DHPR, PTS); Polycystic kidney and hepatic disease (FCYT, PKHD1, ARPKD, PKD2, PKD4, PKDTS, PRKCSH, G19P1, PCLD, SEC63).

Muscular/Skeletal Disorders: Genes linked to these disorders: Becker muscular dystrophy (DMD, BMD, MYF6), Duchenne Muscular Dystrophy (DMD, BMD); Emery-Dreifuss muscular dystrophy (LMNA, LMN1, EMD2, FPLD, CMD1A, HGPS, LGMD1B, LMNA, LMN1, EMD2, FPLD, CMD1A); Facioscapulohumeral muscular dystrophy (FSHMD1A, FSHD1A); Muscular dystrophy (FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, EBS1); Osteopetrosis (LAPS, BMND1, LRP7, LR3, OPPG, VBCH2, CLCN7, CLC7, OPTA2, OSTM1, GL, TCIRG1, TIRC7, 0C116, OPTB1); Muscular atrophy (VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1).

Neurological and Neuronal disorders: Genes linked to these disorders: ALS (SOD1, ALS2, STEX, FUS, TARDBP, VEGF (VEGF-a, VEGF-b, VEGF-c); Alzheimer disease (APP, AAA, CVAP, AD1, APOE, AD2, PSEN2, AD4, STM2, APBB2, FE65L1, NOS3, PLAU, URK, ACE, DCPI, ACEI, MPO, PACIP1, PAXIPIL, PTIP, A2M, BLMH, BMH, PSEN1, AD3); Autism (Mecp2, BZRAP I, MDGA2, Sema5A, Neurex 1, GLO1, MECP2, RTT, PPMX, MRX16, MRX79, NLGN3, NLGN4, KIAA1260, AUTSX2); Fragile X Syndrome (FMR2, FXR1, FXR2, mGLUR5); Huntington's disease and disease like disorders (HD, IT15, PRNP, PRIP, JPH3, JP3, HDL2, TBP, SCA17); Parkinson disease (NR4A2, NURR1, NOT, TINUR, SNCAIP, TBP, SCA17, SNCA, NACP, PARK1, PARK4, DJ1, PARK7, LRRK2, PARKS, PINK1, PARK6, UCHL1, PARKS, SNCA, NACP, PARK1, PARK4, PRKN, PARK-2, PDJ, DBH, NDUFV2); Rett syndrome (MECP2, RTT, PPMX, MRX16, MRX79, CDKL5, STK9, MECP2, RTT, PPMX, MRX16, MRX79, x-Synuclein, DJ-1); Schizophrenia (Neuregulin1 (Nrg1), Erb4 (receptor for Neuregulin), Complexin1 (Cp1x1), Tph1 Tryptophan hydroxylase, Tph2, Tryptophan hydroxylase 2, Neurexin 1, GSK3, GSK3a, GSK3b, 5-HTT (S1c6a4), COMT, DRD (Drd 1a), SLC6A3, DADA, DTNBP1, Dao (Dao1)); Secretase Related Disorders (APH-1 (alpha and beta), Preseni I in (Psen1), nicastrin, (Ncstn), PEN-2, Nos1, Parp1, Nat1, Nat2); Trinucleotide Repeat Disorders (HTT (Huntington's Dx), SBMA/SMAX1/AR (Kennedy's Dx), FXN/X25 (Friedrich's Ataxia), ATX3 (Machado-Joseph's Dx), ATXN1 and ATXN2 (spinocerebellar ataxias), DMPK (myotonic dystrophy), Atrophin-1 and Atnl (DRPLA Dx), CBP (Creb-BP—global instability), VLDLR (Alzheimer's), Atxn7, Atxn10).

Occular-related disorders: Genes linked to these disorders: Age-related macular degeneration (Aber, Cc12, Cc2, cp (ceruloplasmin), Timp3, cathepsinD, Vld1r, Ccr2); Cataract (CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, KRIT1); Corneal clouding and dystrophy (APOA1, TGFBI, CSD2, CDGG1, CSD, BIGH3, CDG2, TACSTD2, TROP2, M1S1, VSX1, RINX, PPCD, PPD, KTCN, COL8A2, FECD, PPCD2, PIP5K3, CFD); Cornea plana congenital (KERA, CNA2); Glaucoma (MYOC, TIGR, GLC1A, JOAG, GPOA, OPTN, GLC1E, FIP2, HYPL, NRP, CYP1B1, GLC3A, OPAL, NTG, NPG, CYP1B1, GLC3A); Leber congenital amaurosis (CRB1, RP12, CRX, CORD2, CRD, RPGRIP1, LCA6, CORDS, RPE65, RP20, AIPL1, LCA4, GUCY2D, GUC2D, LCA1, CORD6, RDH12, LCA3); Macular dystrophy (ELOVL4, ADMD, STGD2, STGD3, RDS, RP7, PRPH2, PRPH, AVMD, AOFMD, VMD2).

P13K/AKT Cellular Signaling disorders: Genes linked to these disorders: PRKCE; ITGAM; ITGA5; IRAK1; PRKAA2; EIF2AK2; PTEN; EIF4E; PRKCZ; GRK6; MAPK1; TSC1; PLK1; AKT2; IKBKB; PIK3CA; CDK8; CDKN1B; NFKB2; BCL2; PIK3CB; PPP2R1A; MAPK8; BCL2L1; MAPK3; TSC2; ITGA1; KRAS; EIF4EBP1; RELA; PRKCD; NOS3; PRKAA1; MAPK9; CDK2; PPP2CA; PIM1; ITGB7; YWHAZ; ILK; TP53; RAF1; IKBKG; RELB; DYRK1A; CDKN1A; ITGB1; MAP2K2; JAK1; AKT1; JAK2; PIK3R1; CHUK; PDPK1; PPP2R5C; CTNNB1; MAP2K1; NFKB1; PAK3; ITGB3; CCND1; GSK3A; FRAP1; SFN; ITGA2; TTK; CSNK1A1; BRAF; GSK3B; AKT3; FOXO1; SOK; HS P9OAA1; RP S 6KB1

ERK/MAPK Cellular Signaling disorders: Genes linked to these disorders: PRKCE; ITGAM; ITGA5; HSPB1; IRAK1; PRKAA2; EIF2AK2; RAC1; RAP1A; TLN1; EIF4E; ELK1; GRK6; MAPK1; RAC2; PLK1; AKT2; PIK3CA; CDK8; CREB1; PRKCI; PTK2; FOS; RPS6KA4; PIK3CB; PPP2R1A; PIK3C3; MAPK8; MAPK3; ITGA1; ETS1; KRAS; MYCN; EIF4EBP1; PPARG; PRKCD; PRKAA1; MAPK9; SRC; CDK2; PPP2CA; PIM1; PIK3C2A; ITGB7; YWHAZ; PPP1CC; KSR1; PXN; RAF1; FYN; DYRK1A; ITGB1; MAP2K2; PAK4; PIK3R1; STAT3; PPP2R5C; MAP2K1; PAK3; ITGB3; ESR1; ITGA2; MYC; TTK; CSNK1A1; CRKL; BRAE; ATF4; PRKCA; SRF; STAT1; SGK

Glucocorticoid Receptor Cellular Signaling disorders: Genes linked to these disorders: RAC1; TAF4B; EP300; SMAD2; TRAF6; PCAF; ELK1; MAPK1; SMAD3; AKT2; IKBKB; NCOR2; UBE2I; PIK3CA; CREB1; FOS; HSPA5; NFKB2; BCL2; MAP3K14; STAT5B; PIK3CB; PIK3C3; MAPK8; BCL2L1; MAPK3; TSC22D3; MAPK10; NRIP1; KRAS; MAPK13; RELA; STAT5A; MAPK9; NOS2A; PBX1; NR3C1; PIK3C2A; CDKN1C; TRAF2; SERPINE1; NCOA3; MAPK14; TNF; RAF1; IKBKG; MAP3K7; CREBBP; CDKN1A; MAP2K2; JAK1; IL8; NCOA2; AKT1; JAK2; PIK3R1; CHUK; STAT3; MAP2K1; NFKB1; TGFBR1; ESR1; SMAD4; CEBPB; JUN; AR; AKT3; CCL2; MMP 1; STAT1; IL6; HSP9OAA1

Axonal Guidance Cellular Signaling disorders: Genes linked to these disorders: PRKCE; ITGAM; ROCK1; ITGA5; CXCR4; ADAM12; IGF1; RAC1; RAP1A; E1 F4E; PRKCZ; NRP1; NTRK2; ARHGEF7; SMO; ROCK2; MAPK1; PGF; RAC2; PTPN11; GNAS; AKT2; PIK3CA; ERBB2; PRKCI; PTK2; CFL1; GNAQ; PIK3CB; CXCL12; PIK3C3; WNT11; PRKD1; GNB2L1; ABL1; MAPK3; ITGA1; KRAS; RHOA; PRKCD; PIK3C2A; ITGB7; GLI2; PXN; VASP; RAF1; FYN; ITGB1; MAP2K2; PAK4; ADAM17; AKT1; PIK3R1; GUI; WNT5A; ADAM10; MAP2K1; PAK3; ITGB3; CDC42; VEGFA; ITGA2; EPHA8; CRKL; RND1; GSK3B; AKT3; PRKCA

Ephrin Recptor Cellular Signaling disorders: Genes linked to these disorders: PRKCE; ITGAM; ROCK1; ITGA5; CXCR4; IRAK1; PRKAA2; EIF2AK2; RAC1; RAP1A; GRK6; ROCK2; MAPK1; PGF; RAC2; PTPN11; GNAS; PLK1; AKT2; DOK1; CDK8; CREB1; PTK2; CFL1; GNAQ; MAP3K14; CXCL12; MAPK8; GNB2L1; ABL1; MAPK3; ITGA1; KRAS; RHOA; PRKCD; PRKAA1; MAPK9; SRC; CDK2; PIM1; ITGB7; PXN; RAF1; FYN; DYRK1A; ITGB1; MAP2K2; PAK4, AKT1; JAK2; STAT3; ADAM10; MAP2K1; PAK3; ITGB3; CDC42; VEGFA; ITGA2; EPHA8; TTK; CSNK1A1; CRKL; BRAF; PTPN13; ATF4; AKT3; SGK

Actin Cytoskeleton Cellular Signaling disorders: Genes linked to these disorders: ACTN4; PRKCE; ITGAM; ROCK1; ITGA5; IRAK1; PRKAA2; EIF2AK2; RAC1; INS; ARHGEF7; GRK6; ROCK2; MAPK1; RAC2; PLK1; AKT2; PIK3CA; CDK8; PTK2; CFL1; PIK3CB; MYH9; DIAPH1; PIK3C3; MAPK8; F2R; MAPK3; SLC9A1; ITGA1; KRAS; RHOA; PRKCD; PRKAA1; MAPK9; CDK2; PIM1; PIK3C2A; ITGB7; PPP1CC; PXN; VIL2; RAF1; GSN; DYRK1A; ITGB1; MAP2K2; PAK4; PIP5K1A; PIK3R1; MAP2K1; PAK3; ITGB3; CDC42; APC; ITGA2; TTK; CSNK1A1; CRKL; BRAF; VAV3; SGK

Huntington's Disease Cellular Signaling disorders: Genes linked to these disorders: PRKCE; IGF1; EP300; RCOR1; PRKCZ; HDAC4; TGM2; MAPK1; CAPNS1; AKT2; EGFR; NCOR2; SP1; CAPN2; PIK3CA; HDAC5; CREB1; PRKC1; HS PA5; REST; GNAQ; PIK3CB; PIK3C3; MAPK8; IGF1R; PRKD1; GNB2L1; BCL2L1; CAPN1; MAPK3; CASP8; HDAC2; HDAC7A; PRKCD; HDAC11; MAPK9; HDAC9; PIK3C2A; HDAC3; TP53; CASP9; CREBBP; AKT1; PIK3R1; PDPK1; CASP1; APAF1; FRAP1; CASP2; JUN; BAX; ATF4; AKT3; PRKCA; CLTC; SGK; HDAC6; CASP3

Apoptosis Cellular Signaling disorders: Genes linked to these disorders: PRKCE; ROCK1; BID; IRAK1; PRKAA2; EIF2AK2; BAK1; BIRC4; GRK6; MAPK1; CAPNS1; PLK1; AKT2; IKBKB; CAPN2; CDK8; FAS; NFKB2; BCL2; MAP3K14; MAPK8; BCL2L1; CAPN1; MAPK3; CASP8; KRAS; RELA; PRKCD; PRKAA1; MAPK9; CDK2; PIM1; TP53; TNF; RAF1; IKBKG; RELB; CASP9; DYRK1A; MAP2K2; CHUK; APAF1; MAP2K1; NFKB1; PAK3; LMNA; CASP2; BIRC2; TTK; CSNK1A1; BRAF; BAX; PRKCA; SGK; CASP3: BTRC3: PARPI

B Cell Receptor Cellular Signaling disorders: Genes linked to these disorders: RAC1; PTEN; LYN; ELK1; MAPK1; RAC2; PTPN11; AKT2; IKBKB; PIK3CA; CREB1; SYK; NFKB2; CAMK2A; MAP3K14; PIK3CB; PIK3C3; MAPK8; BCL2L1; ABL1; MAPK3; ETS1; KRAS; MAPK13; RELA; PTPN6; MAPK9; EGR1; PIK3C2A; BTK; MAPK14; RAF1; IKBKG; RELB; MAP3K7; MAP2K2; AKT1; PIK3R1; CHUK; MAP2K1; NFKB1; CDC42; GSK3A; FRAP1; BCL6; BCL10; JUN; GSK3B; ATF4; AKT3; VAV3; RPS6KB1

Leukocyte Extravasation Cellular Signaling disorders: Genes linked to these disorders: ACTN4; CD44; PRKCE; ITGAM; ROCK1; CXCR4; CYBA; RAC1; RAP1A; PRKCZ; ROCK2; RAC2; PTPN11; MMP14; PIK3CA; PRKCI; PTK2; PIK3CB; CXCL12; PIK3C3; MAPK8; PRKD1; ABL1; MAPK10; CYBB; MAPK13; RHOA; PRKCD; MAPK9; SRC; PIK3C2A; BTK; MAPK14; NOX1; PXN; VIL2; VASP; ITGB1; MAP2K2; CTNND1; PIK3R1; CTNNB1; CLDN1; CDC42; FUR; ITK; CRKL; VAV3; CTTN; PRKCA; MMP1; MMP9

Integrin Cellular Signaling disorders: Genes linked to these disorders: ACTN4; ITGAM; ROCK1; ITGA5; RAC1; PTEN; RAP1A; TLN1; ARHGEF7; MAPK1; RAC2; CAPNS1; AKT2; CAPN2; PIK3CA; PTK2; PIK3CB; PIK3C3; MAPK8; CAV1; CAPN1; ABL1; MAPK3; ITGA1; KRAS; RHOA; SRC; PIK3C2A; ITGB7; PPP1CC; ILK; PXN; VASP; RAF1; FYN; ITGB1; MAP2K2; PAK4; AKT1; PIK3R1; TNK2; MAP2K1; PAK3; ITGB3; CDC42; RND3; ITGA2; CRKL; BRAF; GSK3B; AKT3

Acute Phase Response Cellular Signaling disorders: Genes linked to these disorders: IRAK1; SOD2; MYD88; TRAF6; ELK1; MAPK1; PTPN11; AKT2; IKBKB; PIK3CA; FOS; NFKB2; MAP3K14; PIK3CB; MAPK8; RIPK1; MAPK3; IL6ST; KRAS; MAPK13; IL6R; RELA; SOCS1; MAPK9; FTL; NR3C1; TRAF2; SERPINE1; MAPK14; TNF; RAF1; PDK1; IKBKG; RELB; MAP3K7; MAP2K2; AKT1; JAK2; PIK3R1; CHUK; STAT3; MAP2K1; NFKB1; FRAP1; CEBPB; JUN; AKT3; IL1R1; IL6

PTEN Cellular Signaling disorders: Genes linked to these disorders: ITGAM; ITGA5; RAC1; PTEN; PRKCZ; BCL2L11; MAPK1; RAC2; AKT2; EGFR; IKBKB; CBL; PIK3CA; CDKN1B; PTK2; NFKB2; BCL2; PIK3CB; BCL2L1; MAPK3; ITGA1; KRAS; ITGB7; ILK; PDGFRB; INSR; RAF1; IKBKG; CASP9; CDKN1A; ITGB1; MAP2K2; AKT1; PIK3R1; CHUK; PDGFRA; PDPK1; MAP2K1; NFKB1; ITGB3; CDC42; CCND1; GSK3A; ITGA2; GSK3B; AKT3; FOXO1; CASP3;

p53 Cellular Signaling disorders: Genes linked to these disorders: RPS6KB1 PTEN; EP300; BBC3; PCAF; FASN; BRCA1; GADD45A; BIRC5; AKT2; PIK3CA; CHEK1; TP53INP1; BCL2; PIK3CB; PIK3C3; MAPK8; THBS 1; ATR; BCL2L1; E2F1; PMAIP1; CHEK2; TNFASF10B; TP73; RB1; HDAC9; CDK2; PIK3C2A; MAPK14; TP53; LRDD; CDKN1A; HIPK2; AKT1; PIK3R1; RAM2B; APAF1; CTNNB1; SIRT1; CCND1; PRKDC; ATM; SFN; CDKN2A; JUN; SNAI2; GSK3B; BAX; AKT3

Aryl Hydrocarbon Receptor Cellular Signaling disorders: Genes linked to these disorders: HSPB1; EP300; FASN; TGM2; RXRA; MAPK1; NQO1; NCOR2; SP1; ARNT; CDKN1B; FOS; CHEK1; SMARCA4; NFKB2; MAPK8; ALDH1A1; ATR; E2F1; MAPK3; NRIP1; CHEK2; RELA; TP73; GSTP1; RB1; SRC; CDK2; AHR; NFE2L2; NCOA3; TP53; TNF; CDKN1A; NCOA2; APAF1; NFKB1; CCND1; ATM; ESR1; CDKN2A; MYC; JUN; ESR2; BAX; IL6; CYP1B1; HSP9OAA1

Xenobiotic Metabolism Cellular Signaling disorders: Genes linked to these disorders: PRKCE; EP300; PRKCZ; RXRA; MAPK1; NQO1; NCOR2; PIK3CA; ARNT; PRKCI; NFKB2; CAMK2A; PIK3CB; PPP2R1A; PIK3C3; MAPK8; PRKD1; ALDH1A1; MAPK3; NRIP1; KRAS; MAPK13; PRKCD; GSTP1; MAPK9; NOS2A; ABCB1; AHR; PPP2CA; FTL; NFE2L2; PIK3C2A; PPARGC1A; MAPK14; TNF; RAF1; CREBBP; MAP2K2; PIK3R1; PPP2R5C; MAP2K1; NFKB1; KEAP1; PRKCA; EIF2AK3; IL6; CYP1B1; HSP9OAA1

SAPL/JNK Cellular Signaling disorders: Genes linked to these disorders: PRKCE; IRAK1; PRKAA2; EIF2AK2; RAC1; ELK1; GRK6; MAPK1; GADD45A; RAC2; PLK1; AKT2; PIK3CA; FADD; CDK8; PIK3CB; PIK3C3; MAPK8; RIPK1; GNB2L1; IRS1; MAPK3; MAPK10; DAXX; KRAS; PRKCD; PRKAA1; MAPK9; CDK2; PIM1; PIK3C2A; TRAF2; TP53; LCK; MAP3K7; DYRK1A; MAP2K2; PIK3R1; MAP2K1; PAK3; CDC42; JUN; TTK; CSNK1A1; CRKL; BRAF; SGK

PPAr/RXR Cellular Signaling disorders: Genes linked to these disorders: PRKAA2; EP300; INS; SMAD2; TRAF6; PPARA; FASN; RXRA; MAPK1; SMAD3; GNAS; IKBKB; NCOR2; ABCA1; GNAQ; NFKB2; MAP3K14; STAT5B; MAPK8; IASI; MAPK3; KRAS; RELA; PRKAA1; PPARGC1A; NCOA3; MAPK14; INSR; RAF1; IKBKG; RELB; MAP3K7; CREBBP; MAP2K2; JAK2; CHUK; MAP2K1; NFKB1; TGFBA1; SMAD4; JUN; IL1R1; PRKCA; IL6; HSP9OAA1; ADIPOO

NF-KB Cellular Signaling disorders: Genes linked to these disorders: IRAK1; EIF2AK2; EP300; INS; MYD88; PRKCZ: TRAF6; TBK1; AKT2; EGFR; IKBKB; PIK3CA; BTRC; NFKB2; MAP3K14; PIK3CB; PIK3C3; MAPK8; RIPK1; HDAC2; KRAS; RELA; PIK3C2A; TRAF2; TLR4: PDGFRB; TNF; INSR; LCK; IKBKG; RELB; MAP3K7; CREBBP; AKT1; PIK3R1; CHUK; PDGFRA; NFKB1; TLR2; BCL10; GSK3B; AKT3; TNFAIP3; IL1R1

Neuregulin Cellular Signaling disorders: Genes linked to these disorders: ERBB4; PRKCE; ITGAM; ITGA5: PTEN; PRKCZ; ELK1; MAPK1; PTPN11; AKT2; EGFR; ERBB2; PRKCI; CDKN1B; STAT5B; PRKD1; MAPK3; ITGA1; KRAS; PRKCD; STAT5A; SRC; ITGB7; RAF1; ITGB1; MAP2K2; ADAM17; AKT1; PIK3R1; PDPK1; MAP2K1; ITGB3; EREG; FRAP1; PSEN1; ITGA2; MYC; NRG1; CRKL; AKT3; PRKCA; HS P9OAA1; RPS6KB1

Wnt and Beta catenin Cellular Signaling disorders: Genes linked to these disorders: CD44; EP300; LRP6; DVL3; CSNK1E; GJA1; SMO;

AKT2; PIN1; CDH1; BTRC; GNAQ; MARK2; PPP2R1A; WNT11; SRC; DKK1; PPP2CA; SOX6; SFRP2: ILK; LEF1; SOX9; TP53; MAP3K7; CREBBP; TCF7L2; AKT1; PPP2R5C; WNT5A; LAPS; CTNNB1; TGFBR1; CCND1; GSK3A; DVL1; APC; CDKN2A; MYC; CSNK1A1; GSK3B; AKT3; SOX2

Insulin Receptor Signaling disorders: Genes linked to these disorders: PTEN; INS; EIF4E; PTPN1; PRKCZ; MAPK1; TSC1; PTPN11; AKT2; CBL; PIK3CA; PRKCI; PIK3CB; PIK3C3; MAPK8; IASI; MAPK3; TSC2; KRAS; EIF4EBP1; SLC2A4; PIK3C2A; PPP1CC; INSR; RAF1; FYN; MAP2K2; JAK1; AKT1; JAK2; PIK3R1; PDPK1; MAP2K1; GSK3A; FRAP1; CRKL; GSK3B; AKT3; FOXO1; SGK; RPS6KB1

IL-6 Cellular Signaling disorders: Genes linked to these disorders: HSPB1; TRAF6; MAPKAPK2; ELK1; MAPK1; PTPN11; IKBKB; FOS; NFKB2: MAP3K14; MAPK8; MAPK3; MAPK10; IL6ST; KRAS; MAPK13; IL6R; RELA; SOCS1; MAPK9; ABCB1; TRAF2; MAPK14; TNF; RAF1; IKBKG; RELB; MAP3K7; MAP2K2; IL8; JAK2; CHUK; STAT3; MAP2K1; NFKB1; CEBPB; JUN; IL1R1; SRF; IL6

Hepatic Cholestasis Cellular Signaling disorders: Genes linked to these disorders: PRKCE; IRAK1; INS; MYD88; PRKCZ; TRAF6; PPARA; RXRA; IKBKB; PRKCI; NFKB2; MAP3K14; MAPK8; PRKD1; MAPK10; RELA; PRKCD; MAPK9; ABCB1; TRAF2; TLR4; TNF; INSR; IKBKG; RELB; MAP3K7; IL8; CHUK; NR1H2; TJP2; NFKB1; ESR1; SREBF1; FGFR4; JUN; IL1R1; PRKCA; IL6

IGF-1 Cellular Signaling disorders: Genes linked to these disorders: IGF1; PRKCZ; ELK1; MAPK1; PTPN11; NEDD4; AKT2; PIK3CA; PRKCI; PTK2; FOS; PIK3CB; PIK3C3; MAPK8; IGF1R; IRS1; MAPK3; IGFBP7; KRAS; PIK3C2A; YWHAZ; PXN; RAF1; CASP9; MAP2K2; AKT1; PIK3R1; PDPK1; MAP2K1; IGFBP2; SFN; JUN; CYR61; AKT3; FOXO1; SRF; CTGF; RPS6KB1

NRF2-mediated Oxidative Stress Response Signaling disorders: Genes linked to these disorders: PRKCE; EP300; SOD2; PRKCZ; MAPK1; SQSTM1; NQO1; PIK3CA; PRKCI; FOS; PIK3CB; PIK3C3; MAPK8; PRKD1; MAPK3; KRAS; PRKCD; GSTP1; MAPK9; FTL; NFE2L2; PIK3C2A; MAPK14; RAF1; MAP3K7; CREBBP; MAP2K2; AKT1; PIK3R1; MAP2K1; PPIB; JUN; KEAP1; GSK3B; ATF4; PRKCA; EIF2AK3; HSP9OAA1

Hepatic Fibrosis/Hepatic Stellate Cell Activation Signaling disorders: Genes linked to these disorders: EDN1; IGF1; KDR; FLT1; SMAD2; FGFR1; MET; PGF; SMAD3; EGFR; FAS; CSF1; NFKB2; BCL2; MYH9; IGF1R; IL6R; RELA; TLR4; PDGFRB; TNF; RELB; IL8; PDGFRA; NFKB1; TGFBR1; SMAD4; VEGFA; BAX; IL1R1; CCL2; HGF; MMP1; STAT1; IL6; CTGF; MMP9

PPAR Signaling disorders: Genes linked to these disorders: EP300; INS; TRAF6; PPARA; RXRA; MAPK1; IKBKB; NCOR2; FOS; NFKB2; MAP3K14; STAT5B; MAPK3; NRIP1; KRAS; PPARG; RELA; STAT5A; TRAF2; PPARGC1A; PDGFRB; TNF; INSR; RAF1; IKBKG; RELB; MAP3K7; CREBBP; MAP2K2; CHUK; PDGFRA; MAP2K1; NFKB1; JUN; IL1R1; HSP9OAA1

Fc Epsilon RI Signaling disorders: Genes linked to these disorders: PRKCE; RAC1; PRKCZ; LYN; MAPK1; RAC2; PTPN11; AKT2; PIK3CA; SYK; PRKCI; PIK3CB; PIK3C3; MAPK8; PRKD1; MAPK3; MAPK10; KRAS; MAPK13; PRKCD; MAPK9; PIK3C2A; BTK; MAPK14; TNF; RAF1; FYN; MAP2K2; AKT1; PIK3R1; PDPK1; MAP2K1; AKT3; VAV3; PRKCA

G-Protein Coupled Receptor Signaling disorders: Genes linked to these disorders: PRKCE; RAP1A; RGS16; MAPK1; GNAS; AKT2; IKBKB; PIK3CA; CREB1; GNAQ; NFKB2; CAMK2A; PIK3CB; PIK3C3; MAPK3; KRAS; RELA; SRC; PIK3C2A; RAF1; IKBKG; RELB; FYN; MAP2K2; AKT1; PIK3R1; CHUK; PDPK1; S TAT3; MAP2K1; NFKB1; BRAF; ATF4; AKT3; PRKCA

Inositol Phosphate Metabolism Signaling disorders: Genes linked to these disorders: PRKCE; IRAK1; PRKAA2; EIF2AK2; PTEN; GRK6; MAPK1; PLK1; AKT2; PIK3CA; CDK8: PIK3CB; PIK3C3; MAPK8: MAPK3; PRKCD; PRKAA1; MAPK9; CDK2; PIM1; PIK3C2A; DYRK1A; MAP2K2; PIP5K1A; PIK3R1; MAP2K1; PAK3; ATM; TTK; CSNK1A1; BRAF; SGK

PDGF Signaling disorders: Genes linked to these disorders: EIF2AK2; ELK1; ABL2; MAPK1; PIK3CA; FOS; PIK3CB; P IK3 C3; MAPK8; CAV1; ABL1; MAPK3; KRAS; SRC; PIK3C2A; PDGFRB; RAF1; MAP2K2; JAK1; JAK2; PIK3R1; PDGFRA; STAT3; SPHK1; MAP2K1; MYC; JUN; CRKL; PRKCA; SRF; STAT1; SPHK2 VEGF Signaling disorders: Genes linked to these disorders: ACTN4; ROCK1; KDR; FLT1; ROCK2; MAPK1; PGF; AKT2; PIK3CA; ARNT; PTK2; BCL2; PIK3CB; PIK3C3; BCL2L1; MAPK3; KRAS; HIF1A; NOS3; PIK3C2A; PXN; RAF1; MAP2K2; ELAVL1; AKT1; PIK3R1; MAP2K1; SFN; VEGFA; AKT3; FOXO1; PRKCA

Natural Killer Cell Signaling disorders: Genes linked to these disorders: PRKCE; RAC1; PRKCZ; MAPK1; RAC2; PTPN11; KIR2DL3; AKT2; PIK3CA; SYK; PRKCI; PIK3CB; PIK3C3; PRKD1; MAPK3; KRAS; PRKCD; PTPN6; PIK3C2A; LCK; RAF1; FYN; MAP2K2; PAK4; AKT1; PIK3R1; MAP2K1; PAK3; AKT3; VAV3; PRKCA

Cell Cycle: Gl/S Checkpoint Regulation Signaling disorders: Genes linked to these disorders: HDAC4; SMAD3; SUV39H1; HDAC5; CDKN1B; BTRC; ATR; ABL1; E2F1; HDAC2; HDAC7A; RB1; HDAC11; HDAC9; CDK2; E2F2; HDAC3; TP53; CDKN1A; CCND1; E2F4; ATM; RBL2; SMAD4; CDKN2A; MYC; NRG1; GSK3B; RBL1; HDAC6

T Cell Receptor Signaling disorders: Genes linked to these disorders: RAC1; ELK1; MAPK1; IKBKB; CBL; PIK3CA; FOS; NFKB2; PIK3CB; PIK3C3; MAPK8; MAPK3; KRAS; RELA, PIK3C2A; BTK; LCK; RAF1; IKBKG; RELB, FYN; MAP2K2; PIK3R1; CHUK; MAP2K1; NFKB1; ITK; BCL10; JUN; VAV3

Death Receptor disorders: Genes linked to these disorders: CRADD; HSPB1; BID; BIRC4; TBK1; IKBKB; FADD; FAS; NFKB2; BCL2; MAP3K14; MAPK8; RIPK1; CASP8; DAXX; TNFRSF10B; RELA; TRAF2; TNF; IKBKG; RELB; CASP9; CHUK; APAF1; NFKB1; CASP2; BIRC2; CASP3; BIRC3

FGF Cell Signaling disorders: Genes linked to these disorders: RAC1; FGFR1; MET; MAPKAPK2; MAPK1; PTPN11; AKT2; PIK3CA; CREB1; PIK3CB; PIK3C3; MAPK8; MAPK3; MAPK13; PTPN6; PIK3C2A; MAPK14; RAF1; AKT1; PIK3R1; STAT3; MAP2K1; FGFR4; CRKL; ATF4; AKT3; PRKCA; HGF

GM-CSF Cell Signaling disorders: Genes linked to these disorders: LYN; ELK1; MAPK1; PTPN11; AKT2; PIK3CA; CAMK2A; STAT5B; PIK3CB; PIK3C3; GNB2L1; BCL2L1; MAPK3; ETS1; KRAS; RUNX1; PIM1; PIK3C2A; RAF1; MAP2K2; AKT1; JAK2; PIK3R1; STAT3; MAP2K1; CCND1; AKT3; STAT1

Amyotrophic Lateral Sclerosis Cell Signaling disorders: Genes linked to these disorders: BID; IGF1; RAC1; BIRC4; PGF; CAPNS1; CAPN2; PIK3CA; BCL2; PIK3CB; PIK3C3; BCL2L1; CAPN1; PIK3C2A; TP53; CASP9; PIK3R1; RAB5A; CASP1; APAF1; VEGFA; BIRC2; BAX; AKT3; CASP3; BIRC3 PTPN1; MAPK1; PTPN11; AKT2; PIK3CA; STAT5B; PIK3CB; PIK3C3; MAPK3; KRAS; SOCS1; STAT5A; PTPN6; PIK3C2A; RAF1; CDKN1A; MAP2K2; JAK1; AKT1; JAK2; PIK3R1; STAT3; MAP2K1; FRAP1; AKT3; STAT1

JAK/Stat Cell Signaling disorders: Genes linked to these disorders: PTPN1; MAPK1; PTPN11; AKT2; PIK3CA; STAT5B; PIK3CB; PIK3C3; MAPK3; KRAS; SOCS1; STAT5A; PTPN6; PIK3C2A; RAF1; CDKN1A; MAP2K2; JAK1; AKT1; JAK2; PIK3R1; STAT3; MAP2K1; FRAP1; AKT3; STAT1

Nicotinate and Nicotinamide Metabolism Cell Signaling disorders: Genes linked to these disorders: PRKCE; IRAK1; PRKAA2; EIF2AK2; GRK6; MAPK1; PLK1; AKT2; CDK8; MAPK8; MAPK3; PRKCD; PRKAA1; PBEF1; MAPK9; CDK2; PIM1; DYRK1A; MAP2K2; MAP2K1; PAK3; NT5E; TTK; CSNK1A1; BRAF; SGK

Chemokine Cell Signaling disorders: Genes linked to these disorders: CXCR4; ROCK2; MAPK1; PTK2; FOS; CFL1; GNAQ; CAMK2A; CXCL12; MAPK8; MAPK3; KRAS; MAPK13; RHOA; CCR3; SRC; PPP1CC; MAPK14; NOX1; RAF1; MAP2K2; MAP2K1; JUN; CCL2; PRKCA

IL-2 Cell Signaling disorders: Genes linked to these disorders: ELK1; MAPK1; PTPN11; AKT2; PIK3CA; SYK; FOS; STAT5B; PIK3CB; PIK3C3; MAPK8; MAPK3; KRAS; SOCS1; STAT5A; PIK3C2A; LCK; RAF1; MAP2K2; JAK1; AKT1; PIK3R1; MAP2K1; JUN; AKT3

Synaptic Long Term Depression Signaling disorders: Genes linked to these disorders: PRKCE; IGF1; PRKCZ; PRDX6; LYN; MAPK1; GNAS; PRKCI; GNAQ; PPP2R1A; IGF1R; PRKD1; MAPK3; KRAS; GRN; PRKCD; NOS3; NOS2A; PPP2CA; YWHAZ; RAF1; MAP2K2; PPP2R5C; MAP2K1; PRKCA

Estrogen Receptor Cell Signaling disorders: Genes linked to these disorders: TAF4B; EP300; CARM1; PCAF; MAPK1; NCOR2; SMARCA4; MAPK3; NRIP1; KRAS; SRC; NR3C1; HDAC3; PPARGC1A; RBM9; NCOA3; RAF1; CREBBP; MAP2K2; NCOA2; MAP2K1; PRKDC; ESR1; ESR2

Protein Ubiquitination Pathway Cell Signaling disorders: Genes linked to these disorders: TRAF6; SMURF1; BIRC4; BRCA1; UCHL1; NEDD4; CBL; UBE2I; BTRC; HSPA5; USP7; USP10; FBXW7; USP9X; STUB1; USP22; B2M; BIRC2; PARK2; USPS; USP1; VHL; HSP9OAA1; BIRC3

IL-10 Cell Signaling disorders: Genes linked to these disorders: TRAF6; CCR1; ELK1; IKBKB; SP1; FOS; NFKB2; MAP3K14; MAPK8; MAPK13; RELA; MAPK14; TNF; IKBKG; RELB; MAP3K7; JAK1; CHUK; STAT3; NFKB1; JUN; IL1R1; IL6

VDR/RXR Activation Signaling disorders: Genes linked to these disorders: PRKCE; EP300; PRKCZ; RXRA; GADD45A; HES1; NCOR2; SP1; PRKCI; CDKN1B; PRKD1; PRKCD; RUNX2; KLF4; YY1; NCOA3; CDKN1A; NCOA2; SPP1; LAPS; CEBPB; FOXO1; PRKCA

TGF-beta Cell Signaling disorders: Genes linked to these disorders: EP300; SMAD2; SMURF1; MAPK1; SMAD3; SMAD1; FOS; MAPK8; MAPK3; KRAS; MAPK9; RUNX2; SERPINE1; RAF1; MAP3K7; CREBBP; MAP2K2; MAP2K1; TGFBR1; SMAD4; JUN; SMAD5

Toll-like Receptor Cell Signaling disorders: Genes linked to these disorders: IRAK1; EIF2AK2; MYD88; TRAF6; PPARA; ELK1; IKBKB; FOS; NFKB2; MAP3K14; MAPK8; MAPK13; RELA; TLR4; MAPK14; IKBKG; RELB; MAP3K7; CHUK; NFKB1; TLR2; JUN

p38 MAPK Cell Signaling disorders: Genes linked to these disorders: HSPB1; IRAK1; TRAF6; MAPKAPK2; ELK1; FADD; FAS; CREB1; DDIT3; RPS6KA4; DAXX; MAPK13; TRAF2; MAPK14; TNF; MAP3K7; TGFBR1; MYC; ATF4; IL1R1; SRF; STAT1

Neurolrophin/TRK Cell Signaling disorders: Genes linked to these disorders: NTRK2; MAPK1; PTPN11; PIK3CA; CREB1; FOS; PIK3CB; PIK3C3; MAPK8; MAPK3; KRAS; PIK3C2A; RAF1; MAP2K2; AKT1; PIK3R1; PDPK1; MAP2K1; CDC42; JUN; ATF4

Other cellular dysfunction disorders linked to a genetic modification are contemplated herein for example, FXR/RXR Activation, Synaptic Long Term Potentiation, Calcium Signaling EGF Signaling, Hypoxia Signaling in the Cardiovascular System, LPS/IL-1 Mediated Inhibition of RXR Function LXR/RXR Activation, Amyloid Processing, IL-4 Signaling, Cell Cycle: G2/M DNA Damage Checkpoint Regulation, Nitric Oxide Signaling in the Cardiovascular System Purine Metabolism, cAMP-mediated Signaling, Mitochondrial Dysfunction Notch Signaling Endoplasmic Reticulum Stress Pathway Pyrimidine Metabolism, Parkinson's Signaling Cardiac & Beta Adrenergic Signaling Glycolysis/Gluconeogenesis Interferon Signaling Sonic Hedgehog Signaling Glycerophospholipid Metabolism, Phospholipid Degradation, Tryptophan Metabolism Lysine Degradation Nucleotide Excision Repair Pathway, Starch and Sucrose Metabolism, Aminosugars Metabolism Arachidonic Acid Metabolism, Circadian Rhythm Signaling, Coagulation System Dopamine Receptor Signaling, Glutathione Metabolism Glycerolipid Metabolism Linoleic Acid Metabolism Methionine Metabolism Pyruvate Metabolism Arginine and Praline Metabolism, Eicosanoid Signaling Fructose and Mannose Metabolism, Galactose Metabolism Stilbene, Coumarine and Lignin Biosynthesis Antigen Presentation Pathway, Biosynthesis of Steroids Butanoate Metabolism Citrate Cycle Fatty Acid Metabolism Glycerophosphol ipid Metabolism, Histidine Metabolism Inositol Metabolism Metabolism of Xenobiotics by Cytochrome p450, Methane Metabolism, Phenylalanine Metabolism, Propanoate Metabolism Selenoamino Acid Metabolism Sphingolipid Metabolism Aminophosphonate Metabolism, Androgen and Estrogen Metabolism Ascorbate and Aldarate Metabolism, Bile Acid Biosynthesis Cysteine Metabolism Fatty Acid Biosynthesis Glutamate Receptor Signaling, NRF2-mediated, Oxidative Stress Response Pentose Phosphate Pathway, Pentose and Glucuronate Interconversions, Retinol Metabolism Riboflavin Metabolism Tyrosine Metabolism Ubiquinone Biosynthesis Valine, Leucine and Isoleucine Degradation Glycine, Serine and Threonine Metabolism Lysine Degradation Pain/Taste, or Mitochondrial Function Developmental Neurology or combinations thereof.

In certain embodiments, compositions and methods of modifying a target polynucleotide in a eukaryotic cell are disclosed. In accordance with these embodiments, engineered chimeric nucleic acid guided nucleases bind to a target polynucleotide to effect cleavage of the target polynucleotide thereby modifying the target polynucleotide, wherein the engineered chimeric nucleic acid guided nuclease system comprises an engineered chimeric nucleic acid guided nuclease complexed with a guide sequence (gRNA) hybridized to a target sequence within the target polynucleotide for improved targeting and editing of the polynucleotide.

In another aspect disclosed herein, methods and compositions are provided for modifying expression of a polynucleotide in a eukaryotic cell of a subject. In some embodiments, compositions and methods include an engineered chimeric nucleic acid guided nuclease system complex capable of binding a target polynucleotide such that binding leads to an in increased or decreased expression of the targeted polynucleotide; wherein the engineered chimeric nucleic acid guided nuclease system complex comprises an engineered chimeric nucleic acid guided nuclease complexed with a guide sequence (gRNA) hybridized to a target sequence within the targeted polynucleotide, wherein the complex is capable of altering expression of the targeted polynucleotide.

In some embodiments, a target polynucleotide of an engineered chimeric nucleic acid guided nuclease system complex can be any polynucleotide endogenous or exogenous to the eukaryotic cell or other cell. In accordance with these embodiments, the target polynucleotide can be a polynucleotide located in the nucleus of the eukaryotic cell. In certain embodiments, the target polynucleotide can be a sequence encoding a gene product (e.g., a protein) or a non-coding sequence (e.g., a regulatory polynucleotide or a junk DNA). In other embodiments, the target sequence is associated with a PAM (protospacer adjacent motif). A PAM is, a short sequence recognized by the engineered chimeric nucleic acid guided nuclease. Sequences and lengths for PAM differ depending on the engineered chimeric nucleic acid guided nuclease used, but PAMs can be 2-5 base pair sequences adjacent a protospacer (that is, the target sequence. Examples of PAM sequences provided herein and in the examples section below. One of skill in the art will be able to identify further PAM sequences for use with a given engineered chimeric nucleic acid guided nuclease of the instant application using known methods.

In certain embodiments, a targeted gene of a genetic disorder can include a genetic disorder of a human or other mammal such as a pet, livestock or other animal. In yet other embodiments, a targeted gene of a genetic disorder can include a genetic plant disorder.

With advances in crop genomics, the ability to use gene-editing systems to perform efficient and cost effective gene editing and manipulation can allow rapid selection and comparison of single and multiplexed genetic manipulations to transform such genomes for improved production and enhanced traits such as drought resistance and resistance to infection, for example.

Some embodiments disclosed herein relate to use of an engineered chimeric nucleic acid guided nuclease system disclosed herein; for example, in order to target and knock out genes, amplify genes and/or repair particular mutations associated with DNA repeat instability and a medical disorder. This chimeric nuclease system may be used to harness and to correct these defects of genomic instability. In other embodiments, engineered chimeric nucleic acid guided nuclease systems disclosed herein can be used for correcting defects in the genes associated with Lafora disease. Lafora disease is an autosomal recessive condition which is characterized by progressive myoclonus epilepsy which may start as epileptic seizures in adolescence. This condition causes seizures, muscle spasms, difficulty walking, dementia, and eventually death.

In yet another aspect of the invention, the engineered chimeric nucleic acid guided nuclease system can be used to correct genetic-eye disorders that arise from several genetic mutations further described in Genetic Diseases of the Eye, Second Edition, edited by Elias I. Traboulsi, Oxford University Press, 2012.

Several further aspects of the invention relate to correcting defects associated with a wide range of genetic diseases which are further described on the website of the National Institutes of Health under the topic subsection Genetic Disorders. Certain genetic disorders of the brain can include, but are not limited to, Adrenoleukodystrophy, Agenesis of the Corpus Callosum, Aicardi Syndrome, Alpers' Disease, glioblastoma, Alzheimer's, Barth Syndrome, Batten Disease, CADASIL, Cerebellar Degeneration, Fabry's Disease, Gerstmann-Straussler-Schei-nker Disease, Huntington's Disease and other Triplet Repeat Disorders, Leigh's Disease, Lesch-Nyhan Syndrome, Menkes Disease, Mitochondrial Myopathies and NINDS Colpocephaly or other brain disorder contributed to by genetically-linked causation.

In some embodiments, a genetically-linked disorder can be a neoplasia. In some embodiments, where the condition is neoplasia, targeted genes can include one or more genes listed above. In some embodiments, a health condition contemplated herein can be Age-related Macular Degeneration or a Schizophrenic-related Disorder. In other embodiments, the condition may be a Trinucleotide Repeat disorder or Fragile X Syndrome. In other embodiments, the condition may be a Secretase-related disorder. In some embodiments, the condition may be a Prion-related disorder. In some embodiments, the condition may be ALS. In some embodiments, the condition may be a drug addiction related to prescription or illegal substances. In accordance with these embodiments, addiction-related proteins may include ABAT for example.

In some embodiments, the condition may be Autism. In some embodiments, the health condition may be an inflammatory-related condition, for example, over-expression of a pro-inflammatory cytokine. Other inflammatory condition-related proteins can include one or more of monocyte chemoattractant protein-1 (MCP1) encoded by the Ccr2 gene, the C C chemokine receptor type 5 (CCR5) encoded by the Ccr5 gene, the IgG receptor IIB (FCGR2b, also termed CD32) encoded by the Fcgr2b gene, or the Fc epsilon Rlg (FCER1g) protein encoded by the Fcerlg gene, or other protein having a genetic-link to these conditions.

In some embodiments, the condition may be Parkinson's Disease. In accordance with these embodiments, proteins associated with Parkinson's disease can include, but are not limited to, a-synuclein, DJ-1, LRRK2, PINK′, Parkin, UCHL1, Synphilin-1, and NURR1.

Cardiovascular-associated proteins that contribute to a cardiac disorder, can include, but are not limited to, IL1β (interleukin 1-beta), XDH (xanthine dehy-drogenase), TP53 (tumor protein p53), PTGIS (prostaglandin 12 (prostacyclin) synthase), MB (myoglobin), IL4 (interleu-kin 4), ANGPT1 (angiopoietin 1), ABCG8 (ATP-binding cassette, sub-family G (WHITE), member 8), or CTSK (cathepsin K), or other known contributors to these conditions.

In some embodiments, the condition may be Alzheimer's disease. In accordance with these embodiments, Alzheimer's disease associated proteins may include very low density lipoprotein receptor protein (VLDLR) encoded by the VLDLR gene, ubiquitin-like modifier activating enzyme 1 (UBA1) encoded by the UBA1 gene, or for example, NEDD8-activating enzyme E1 catalytic subunit protein (UBE1C) encoded by the UBA3 gene or other genetically-related contributor.

In some embodiments, the condition may be an Autism Spectrum Disorder. In accordance with these embodiments, proteins associated Autism Spectrum Disorders can include the benzodiazapine receptor (peripheral) associated protein 1 (BZRAP1) encoded by the BZRAP1 gene, the AF4/FMR2 family member 2 protein (AFF2) encoded by the AFF2 gene (also termed MFR2), the fragile X mental retardation autosomal homolog 1 protein (FXR1) encoded by the FXR1 gene, or the fragile X mental retardation autosomal homolog 2 protein (FXR2) encoded by the FXR2 gene, or other genetically-related contributor.

In some embodiments, the condition may be Macular Degeneration. In accordance with these embodiments, proteins associated with Macular Degeneration can include, but are not limited to, the ATP-binding cassette, sub-family A (ABC1) member 4 protein (ABCA4) encoded by the ABCR gene, the apolipoprotein E protein (APOE) encoded by the APOE gene, or the chemokine (CC motif) Llg and 2 protein (CCL2) encoded by the CCL2 gene, or other genetically-related contributor.

In some embodiments, the condition may be Schizophrenia. In accordance with these embodiments, proteins associated with Schizophrenia In accordance with these embodiments, proteins associated with Schizophrenia y include NRG1, ErbB4, CPLX1, TPH1, TPH2, NRXN1, GSK3A, BDNF, DISCI, GSK3B, and combinations thereof.

In some embodiments, the condition may be tumor suppression. In accordance with these embodiments, proteins associated with tumor suppression can include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, or Notch 4 or other genetically-related contributor.

In some embodiments, the condition may be a secretase disorder. In accordance with these embodiments, proteins associated with a secretase disorder can include PSENEN (presenilin enhancer 2 homolog (C. elegans)), CTSB (cathepsin B), PSEN1 (presenilin 1), APP (amyloid beta (A4) precursor protein), APH1B (anterior pharynx defective 1 homolog B (C. elegans)), PSEN2 (presenilin 2 (Alzheimer disease 4)), or BACE1 (beta-site APP-cleaving enzyme 1), or other genetically-related contributor.

In some embodiments, the condition may be Amyotrophic Lateral Sclerosis. In accordance with these embodiments, proteins associated with can include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof or other genetically-related contributor.

In some embodiments, the condition may be a prion disease disorder. In accordance with these embodiments, proteins associated with a prion diseases disorder can include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof or other genetically-related contributor. Examples of proteins related to neurodegenerative conditions in prion disorders can include A2M (Alpha-2-Macro-globulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), or ADRA1D (Alpha-1D adrenergic receptor for Alpha-1D adrenoreceptor), or other genetically-related contributor.

In some embodiments, the condition may be an immunodeficiency disorder. In accordance with these embodiments, proteins associated with an immunodeficiency disorder can include A2M [alpha-2-macroglobulin]; AANAT [aryla-lkylamine N-acetyltransferase]; ABCA1 [ATP-binding cassette, sub-family A (ABC1), member 1]; ABCA2 [ATP-binding cassette, sub-family A (ABC1), member 2]; or ABCA3 [ATP-binding cassette, sub-family A (ABC 1), member 3]; or other genetically-related contributor.

In some embodiments, the condition may be an immunodeficiency disorder. In accordance with these embodiments, proteins associated with an immunodeficiency disorder can include Trinucleotide Repeat Disorders include AR (androgen receptor), FMR1 (fragile X mental retardation 1), HTT (huntingtin), or DMPK (dystro-phia myotonica-protein kinase), FXN (frataxin), ATXN2 (ataxin 2), or other genetically-related contributor.

In some embodiments, the condition may be a Neurotransmission Disorders. In accordance with these embodiments, proteins associated with a Neurotransmission Disorders can include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), or HTR2c (5-hydrox-ytryptamine (serotonin) receptor 2C), or other genetically-related contributor. In other embodiments, neurodevelopmental-associated sequences can include, but are not limited to, A2BP1 [ataxin 2-binding protein 1], AADAT [aminoadipate aminotransferase], AANAT [arylalkylamine N-acetyltransferase], ABAT [4-aminobutyrate aminotrans-ABCA1 [ATP-binding cassette, sub-family A (ABC1), member 1], or ABCA13 [ATP-binding cassette, sub-family A (ABC1), member 13], or other genetically-related contributor.

In yet other embodiments, genetic health conditions can include, but are not limited to Aicardi-Goutieres Syndrome; Alexander Disease; Allan-Herndon-Dudley Syndrome; POLG-Related Disorders; Alpha-Mannosidosis (Type II and III); Alstrom Syndrome; Angelman; Syndrome; Ataxia-Telangiectasia; Neuronal Ceroid-Lipofuscinoses; Beta-Thalassemia; Bilateral Optic Atrophy and (Infantile) 3 Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; 4 Friedreich Ataxia [FRDA]; Fryns Syndrome; Fucosidosis; Fukuyama Congenital Muscular Dystrophy; Galactosialido-sis; Gaucher Disease; Organic Acidemias; Hemophagocytic Lymphohistiocytosis; Hutchinson-Gilford Progeria Syndrome; Mucolipidosis II; Infantile Free Sialic Acid Storage 4 Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIST-Associated Lissen-5 cephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accu¬mulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodys-plasia Punctata Type 1; Roberts Syndrome; Sandhoff Disease; Schindler Disease Type 1; Adenosine Deaminase Deficiency; Smith-Lemli-Opitz Syndrome; Spinal Muscular Atrophy; Infantile-Onset Spinocerebellar Ataxia; Hex-osaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; and Xeroderma Pigmentosum.

In other embodiments, genetic disorders in animals targeted by editing systems disclosed herein can include, but are not limited to, Hip Dysplasia, Urinary Bladder conditions, epilepsy, cardiac disorders, Degenerative Myelopathy, Brachycephalic Syndrome, Glycogen Branching Enzyme Deficiency (GBED), Hereditary Equine Regional Dermal Asthenia (HERDA), Hyperkalemic Periodic Paralysis Disease (HYPP), Malignant Hyperthermia (MH), Polysaccharide Storage Myopathy—Type 1 (PSSM1), junctional epdiermolysis bullosa, cerebellar abiotrophy, lavender foal syndrome, fatal familial insomnia, or other animal-related genetic disorder.

As will be apparent, it is envisaged that the present system can be used to target any polynucleotide sequence of interest. Some examples of conditions or diseases that might be use fully treated using the present system are included in the Tables above and examples of genes currently associated with those conditions are also provided there. However, the genes exemplified are not exhaustive.

It is contemplated herein that compositions containing the engineered chimeric nucleic acid guided nucleases SEQ ID NO: 1 to 9 and/or the encoded polypeptide thereof. In certain embodiments, kits contemplated herein can be of use in methods of targeted gene editing. Kits contemplated herein can include at least one container and other reagents combined or in separate containers. Other compositions can be included in the kit such as a composition containing a gRNA or other required components.

In some embodiments, the engineered chimeric nucleic acid guided nuclease protein is codon optimized for expression in the eukaryotic cell.

Additional objects, advantages, and novel features of this disclosure will become apparent to those skilled in the art upon review of the following examples in light of this disclosure. The following examples are not intended to be limiting.

### EXAMPLES

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes can be made in the embodiments which are disclosed and still obtain a like or similar result without departing from the spirit and scope of the disclosure.

### Example 1

In one exemplary method, several different wild-type Cas12 as were used to generate chimeras of the instantly claimed inventions including chimeric Cas12a constructions having a nucleic acid sequence represented by SEQ ID NO:1 to SEQ ID NO:9 or polypeptide encoded by one or more of the nucleic acid represented by SEQ ID NO:1 to SEQ ID NO:9. In certain methods, many different Cas12a nucleases (e.g. nine different Cas12a nucleases) were used as templates for constructing chimeric constructs disclosed herein. The Cas12a nucleases were cleaved 5′ of these recognition sites in certain exemplary methods to construct designer non-naturally occurring chimeric Cas12a constructs with conserved genome editing capabilities.

In other methods, a control Cas12a was used to assess Cas12a genome editing capabilities of the engineered chimeric nucleic acid guided nucleases. The control was used as a comparison template where one and in some cases two cleavages were made in the control sequence. For example, a Cas12a chimeric construct was introduced into a plasmid having lambda red proteins. Cas12a nuclease contains a temperature sensitive inducible promoter. The lambda red proteins of the plasmid used in these recombineering techniques have an arabinose inducible promoter. Then, the engineered chimeric nucleic acid guided nucleases were introduced into a plasmid library in a bacterial culture (e.g., E. coli strain MG1655). Following this process, a second plasmid (e.g., a gRNA plasmid) was introduced to the bacterial culture. This second plasmid targets the galK gene (e.g. knocking out this galK gene) on the E. coli genome. It was demonstrated that the designer chimeric constructs in the tested bacterial cultures created two phenotypes when the strain contained a chimera having genome editing capabilities: 1) the E. coli is capable of growing on the 2-DOG media; and 2) the E. coli colony is white in color on MacConkey agar. It was demonstrated that these chimeric constructs in the tested bacterial cultures created two phenotypes when the strain contained a chimera not having genome editing capabilities: 1) the E. coli is unable to grow on the 2-DOG media and 2) the E. coli colony is red in color on the MacConkey agar. Therefore, these easily distinguishable phenotypes were used to demonstrate E. coli having editing or not having editing capabilities, for screening and selecting for genome-editing/functional chimera Cas12a constructs.

In certain methods, these 2-DOG selection methods were used to readily identify genome-editing/functional chimera Cas12a constructs. With these methods, a gal-off color screening method (on the MacConkey agar) was used wherein editing efficiency of chimera Cas12a construct was calculated.

### Example 2

In other exemplary methods, kanamycin-containing plasmid constructs containing PAM_testing cassettes libraries were created for assessing genome editing specificity and efficiency. For these libraries, each plasmid contained the same spacer but different PAM sites for Cas12a. The designer chimeric Cas12a constructs were introduced to test genome-editing capabilities of the constructs when in the presence of the gRNA targeting having the same spacer as the PAM_testing cassettes library. In these experiments, if the E. coli cells cannot grow on a kanamycin-containing media, then the PAM on the kanamycin plasmid is a functional PAM, recognized by the designer chimeric construct. Alternatively, if the E. coli cells can grow on the kanamycin media, then the PAM on the kanamycin plasmid is a non-functional PAM and the designer chimeric construct is incapable of performing Cas12a genome editing.

In certain methods, chimeric constructs created by strategies disclosed herein were selected based on criteria referenced above where the chimeric construct created grew on 2-DOG media but was white in color on MacConkey agar. These designer chimeric nucleases were selected and further analyzed for improved editing, for example, reduced off-targeting rates and PAM recognition criteria.

### ADDITIONAL DESCRIPTIVE EMBODIMENTS AND EXAMPLES

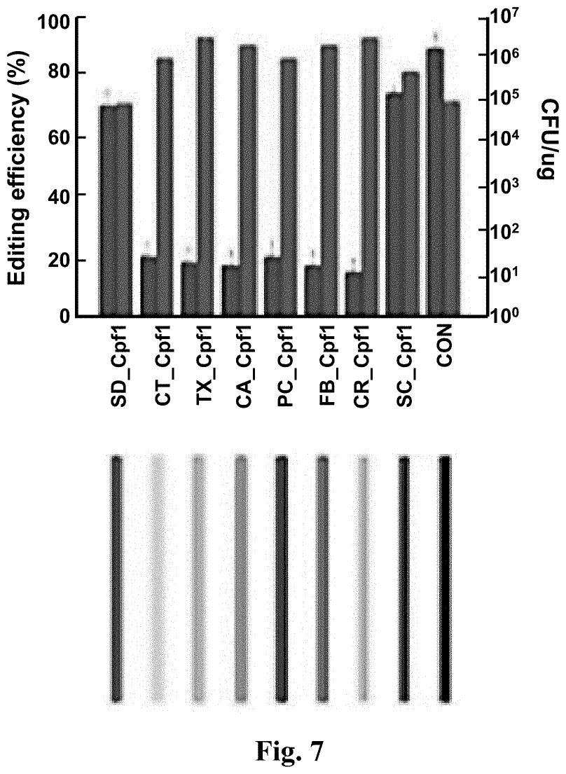

FIG. 7 illustrates editing efficiency of certain constructs disclosed herein.

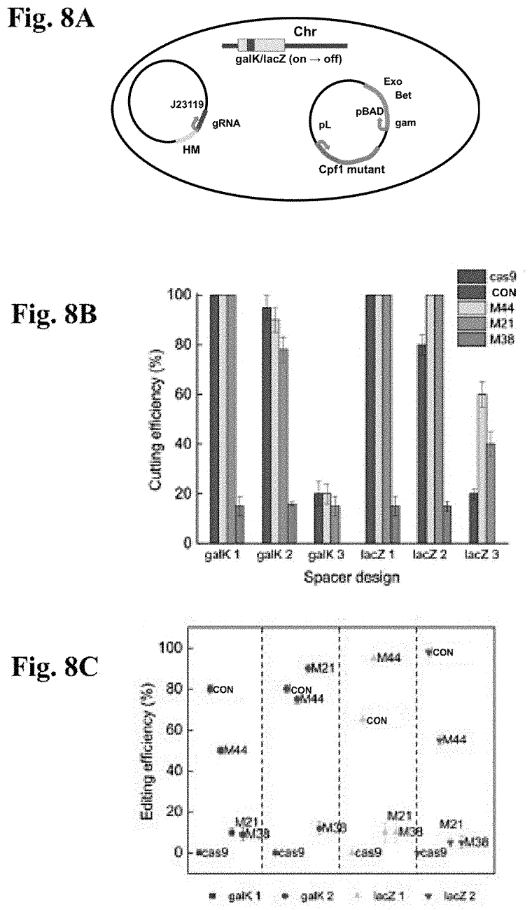

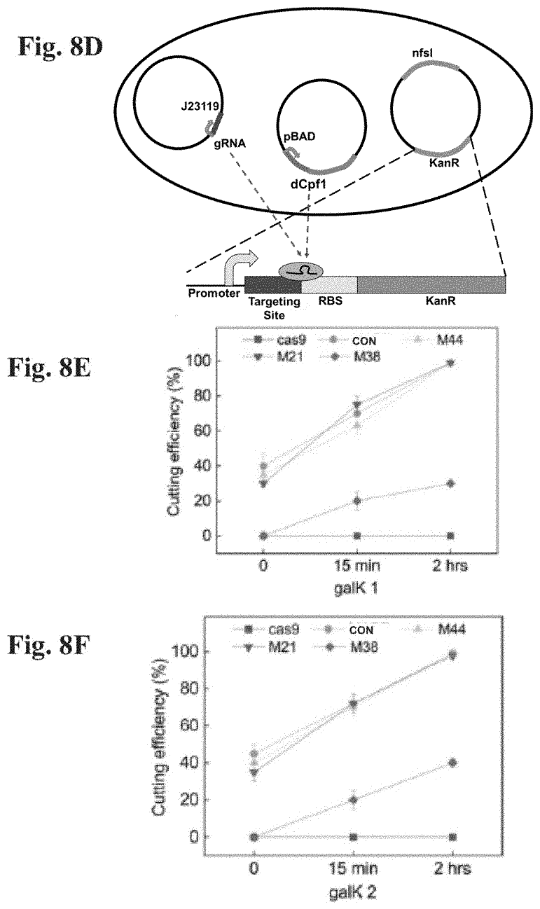

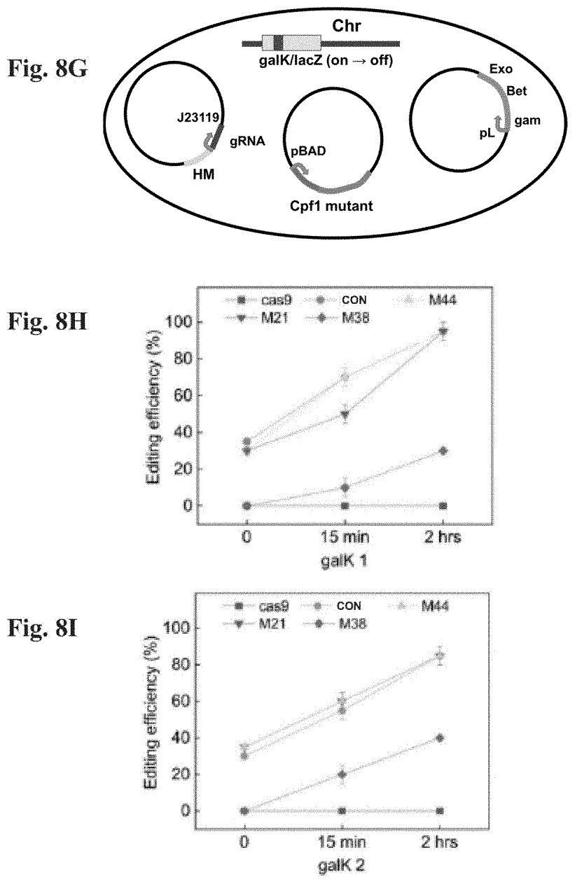

FIGS. 8A-8I: Genome editing test with different gRNAs for chimera library variants in bacteria (e.g. E. coli) (8A) Editing (cutting) efficiency test using gRNA targeting galK or lacZ genes. In certain exemplary methods two plasmid system constructs were created for genome editing: one plasmid expresses a Cas protein as well as lambda red proteins (exo, bet, and gam)⁶⁶; a second plasmid expresses a single crRNA (with J23119 promoter) targeting the galK or lacZ gene and a homology arm (HM) containing a gene-inactivating mutation. For cutting, there were no lambda red proteins or homology arm in the system. (8B) illustrates a histogram plot of cutting efficiency of chimeric Cas12a like proteins using 6 different gRNA plasmids. In this example, gRNA plasmids galK1, galK2, and galK3 targeted different positions in the galK gene. Further, gRNA plasmids lacZ1, lacZ2, and lacZ3 targeted different positions in the lacZ gene. In 8C, editing efficiency of chimera library variants with different gRNAs was examined. In these examples, the gRNAs used in the test were galK1, galK2, lacZ1, and lacZ2. Editing efficiency can be determined by color screening for quick analysis, for example red/white for GalK or blue/white for LacZ. A subset of colonies were sequenced to verify that the edit took place and to assess editing. In 8D, dCas12a (or Cas12a with reduced activity) was evaluated in a protein binding assay. In this exemplary method, three plasmid systems were designed: one plasmid expresses dCas12a (or Cas12a with reduced activity) using an arabinose inducible promoter (pBAD); a second plasmid expresses a single crRNA (with J23119 promoter) targeting the kanR gene; and a third plasmid expresses the kanamycin resistance protein (encoded by kanR gene) using a constitutive promoter containing a fully complementary (on-target) crRNA binding site as well as a nitroreductase (encoded by nfsI gene) which makes the cells sensitive to metronidazole. (8E and 8F) Cutting efficiency of chimeric Cas12a like nucleases with different arabinose induction times using different gRNA were analyzed. (8E) galK_1 and (8F) galK_2. 8G represents a schematic of the system used for testing various Cas12a-like chimera nucleases and controls. In certain methods, an arabinose inducible system for chimeric Cas12a-like proteins was used. In this example, three novel plasmid systems were created for testing genome editing: one plasmid expresses a Cas12a-like protein using an arabinose inducible promoter; a second plasmid expresses lambda red proteins (exo, bet, and gam) using a temperature-inducible promoter (pL); and a third plasmid expresses a single crRNA (with J23119 promoter) targeting the galK gene with homology arm (HM) containing a ga/K-inactivating mutation as a template for recombineering. (8H and 8I) Editing efficiency of chimeric Cas12a like nucleases with different arabinose induction times using different gRNA were analyzed and are represented by 8H: galK_1 and 8I: galK_2.

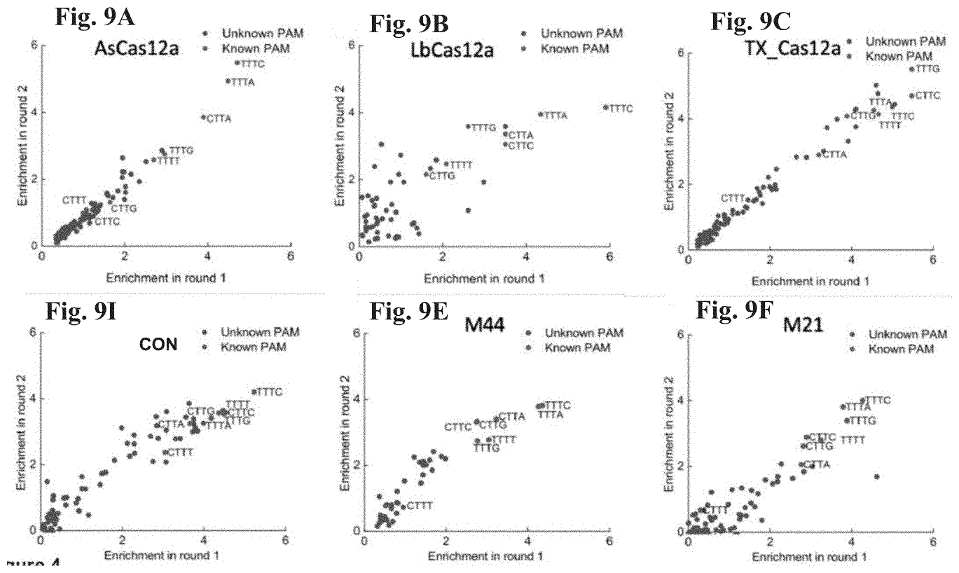

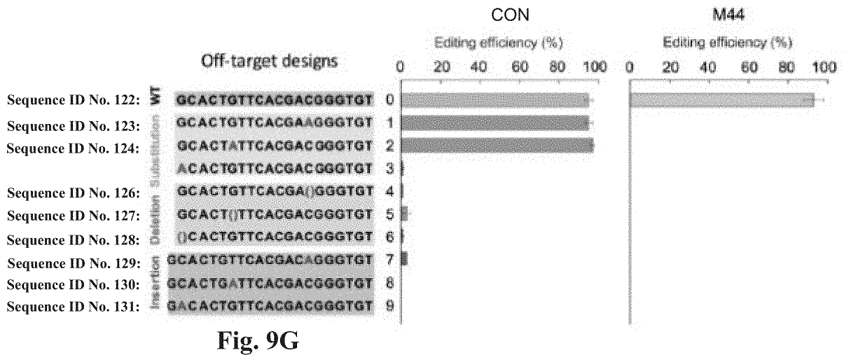

FIGS. 9A-9F represents specificity detection of chimeric Cas12a-type variants and enrichment scoring of each PAM site using different guide RNAs. (9A-9F) Round 1 is illustrated of enrichment scores for two rounds of PAM scans. The enrichment score is the frequency change (log 2) of each PAM using different gRNA plasmids (on-targeting and non-targeting gRNAs). (9A) AsCas12a (9B) LbCas12a (9C) TX_Cas12a (9D) Control (9E) M44 (9F) M21.

FIG. 9G illustrates an off-target assay for chimeric Cas12a-type variants. 9G represents an individual off-target assay. 9 different off-target spacers were designed as illustrated to test editing efficiency and target recognition, of which 3 were substitutions, 3 were deletions, and 3 were insertions. (data not shown) Genome-wide off-target analysis was done using one method referenced as the CIRCLE-seq method. gRNA targeting the galK1 site and gRNA targeting the lacZ2 site were assessed (data not shown). Positions with mismatches to the target sequences, i.e. off-target sites, are highlighted in color. CIRCLE-seq read counts are shown to the right of the on- and off-target sequences and represent a measure of cleavage efficiency at a given site. The on/off-target reads shown in the figure were higher than 10.

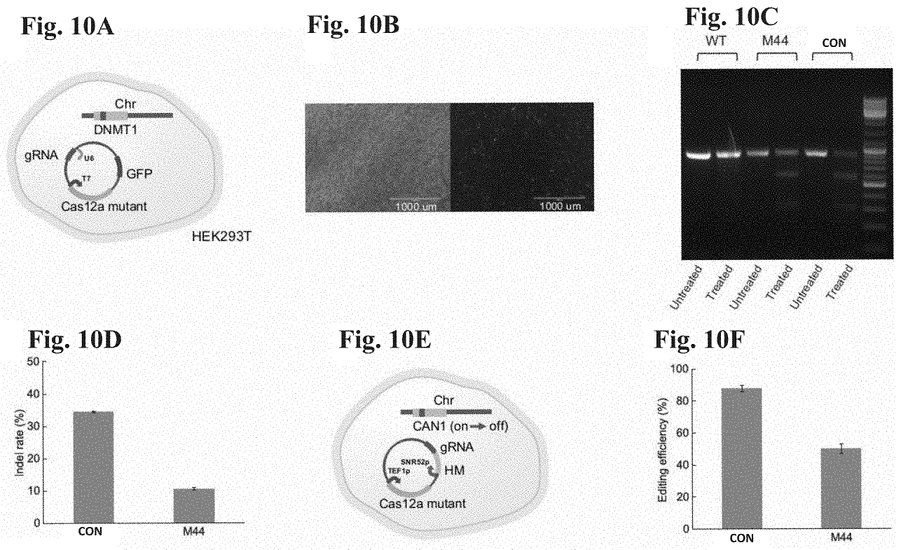

FIGS. 10A-10F In certain exemplary methods, chimeric Cas12a-like nucleases disclosed herein are capable of genome editing in eukaryotic cells. In one method, genome editing in mammalian cells (e.g. HEK293T) were analyzed using chimeric Cas12a-like variants disclosed in certain embodiments herein. A plasmid expressing the M44 (or control) nuclease (with T7 promoter), a single crRNA (with U6 promoter), and GFP were constructed (10A). FIG. 10B is a photographic representation of the mammalian cells after transfection. The mammalian cells were transfected with the plasmid containing the chimeric Cas12a (e.g. M44) nuclease and GFP. Micrographs were taken under cool white light (left) or fluorescent light (right). The T7E1 assay was performed as known in the art on cells expressing GFP and isolated by fluorescence activated cell sorting. In this example, ‘Untreated’ as labeled means the PCR products without T7 endonuclease treatment; while ‘Treated’ means the PCR products with T7 endonuclease treatment (10C). 10D is a graphic representation of an indel rate of control versus the chimeric nuclease, M44. This calculation was made using the formula illustrated in the methods section. 10E represents assessment of genome editing in yeast (S. cerevisiae BY4741) using chimeric Cas12a-type variants as another example of the diversity of organism applicability. In this example, a plasmid was constructed containing the M44 (or control) nuclease (with TEFlp promoter), a single crRNA (SNR52p promoter) targeting the CAM gene and a homology arm (HM) containing a CAN1-inactivating mutation as a template for recombineering. Only colonies with an inactivated CAN1 gene can grow on a +can plate. 10F is a graphic illustration of editing efficiency of control and the tested chimera Cas12a-like nuclease, M44. The editing efficiency was calculated by determining the ratio of colonies on plates+/−can. Editing was also confirmed by sequencing 20 colonies from +can plates.

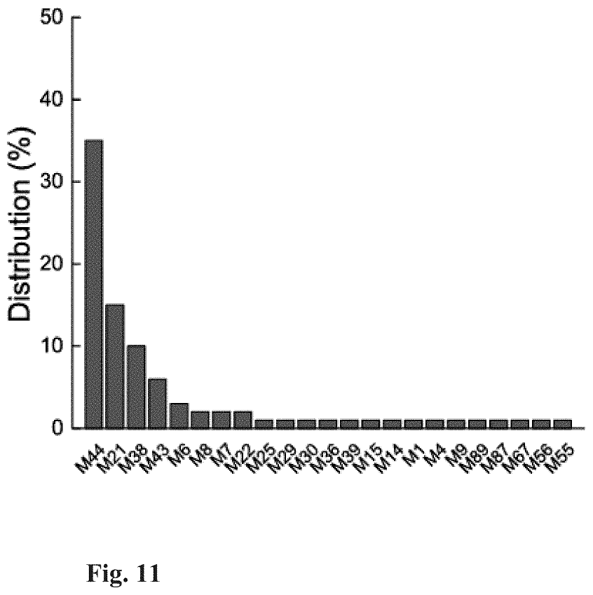

FIG. 11 is an exemplary graph illustrating distribution of functional chimera Cas12a-like nucleases identified using a selection assay (e.g. 2-DOG) of certain embodiments disclosed herein.

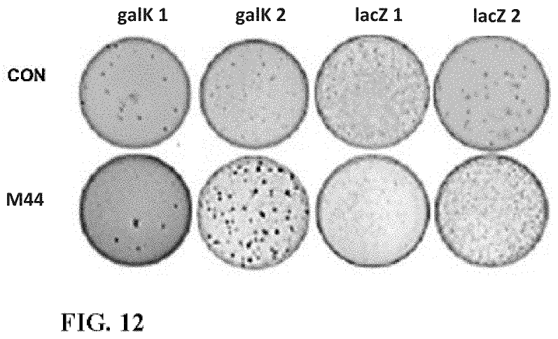

FIG. 12 illustrates a color screening of control versus a chimera Cas12a-like nuclease (e.g. M44) with different gRNAs. The edited cells in the galK/lacZ color screening should be shown as white color. The unedited cells in the galK/lacZ color screening should be shown as red color.

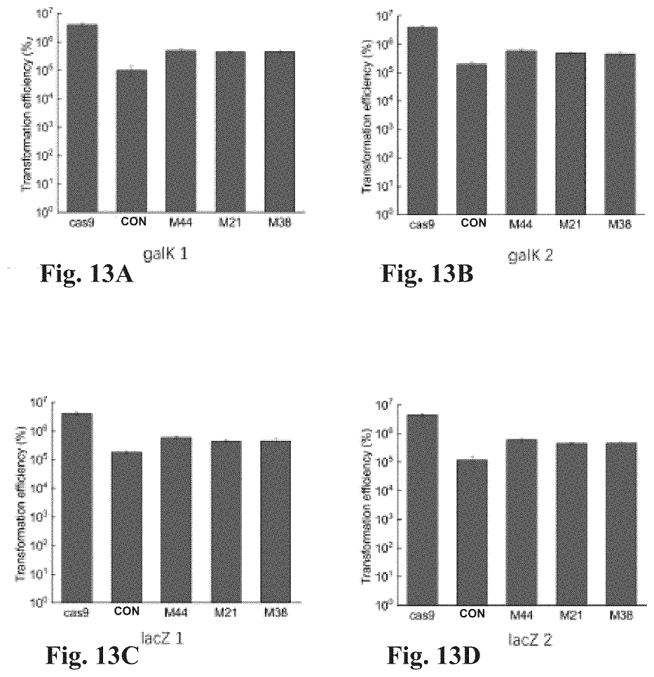

FIGS. 13A-13D illustrate exemplary histogram plots that represent transformation efficiency of different Cas12a-like chimera variants using different gRNA. The gRNA used in the test were (13A) galK1 (13B) galK2 (13C) lacZ1 and (13D) lacZ2. Transformation efficiency is defined as the number of colony forming units (cfu) per μg of gRNA plasmid.

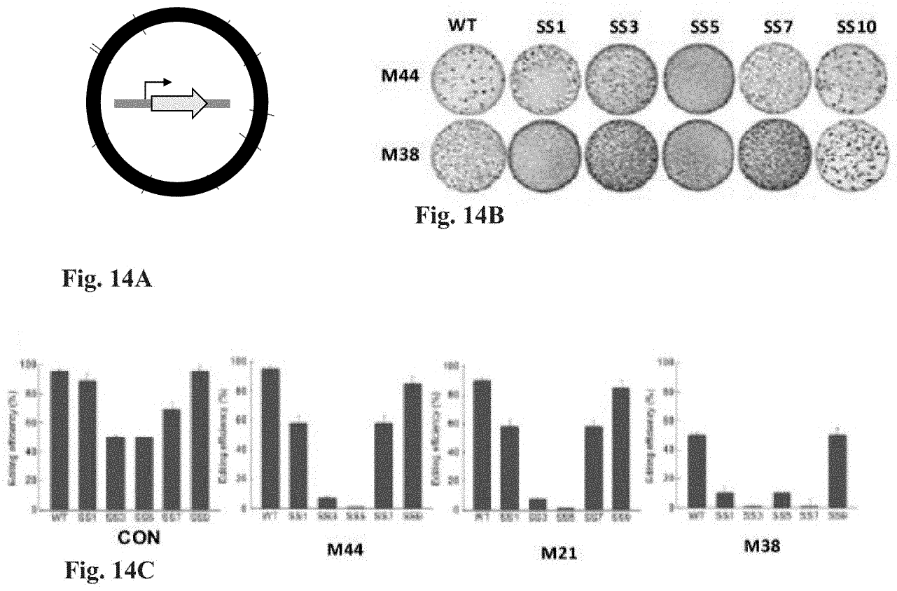

FIGS. 14A-14C illustrate genome editing tests in the different genomic positions for chimera Cas12a-like library variants. 14A illustrates a schematic of targeted genomic position. galK gene was integrated individually in the different genomic position (SS1, SS3, SSS, SS7, and SS9) of MG1655AgalK. 14B illustrates representative plates for colorimetric screening of GalK activity with chimera nuclease variants M44 and M38 in different genomic position. 14C illustrates editing efficiency of chimera library variants in different genomic positions.

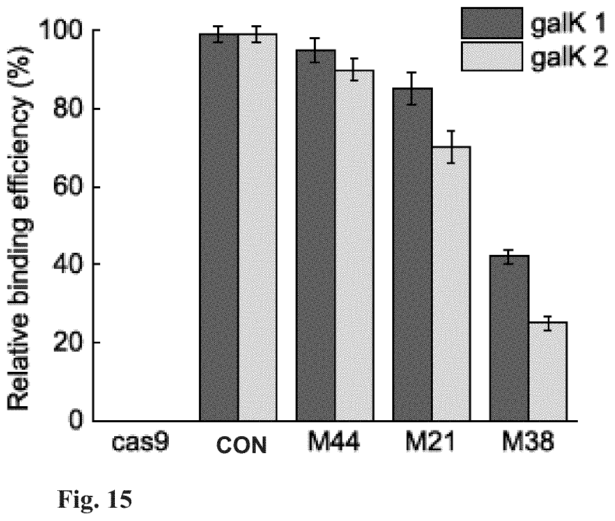

FIG. 15 represents a histogram plot of binding efficiency of dCas12a using different guide RNAs (e.g. galK_1 and galK_2). The binding efficiency was calculated by the following formula.

$$DNA binding efficiency = ( 1 - \frac{Cells in the LB agar plate with kanamycin}{Cells in the LB agar plate without kanamycin} ) \times 100 \%$$

In certain methods, PAM scan methods were designed to assess on and off-targeting rates. Reporter plasmids were constructed containing KanR gene encoding kanamycin resistance and the functional protospacer with NNNN PAM library. The chimera Cas12a-like proteins were transformed and one of two gRNA plasmids were also transformed individually into the E. coli MG1655. One gRNA design is targeted on the KanR gene, and another gRNA plasmid is non-targeting control. These two gRNA plasmids were equivalent amount for the transformation. Cells grown on kanamycin media were collected using different gRNA plasmids, and amplified the region of the PAM library from the reported plasmid for the high throughput sequencing. The enrichment score of PAM and accompanying sequence logo for one of two library replicates revealed the PAM specificity among different chimera Cas12a like proteins. A first round PAM scan tests different variants. (b) AsCas12a (c) LbCas12a (d) TX_Cas12a (e) MAD7 (f) M44 (g) M21 (h) M38 and then plotted where the X- and Y-axis were normalized reads frequency (data not shown).

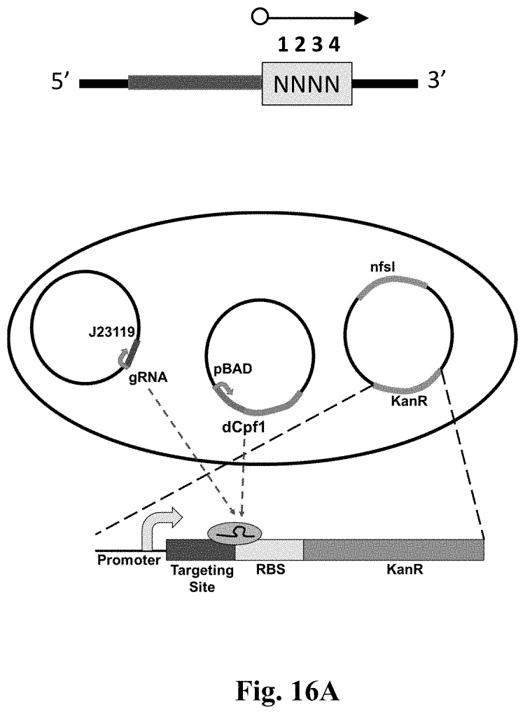

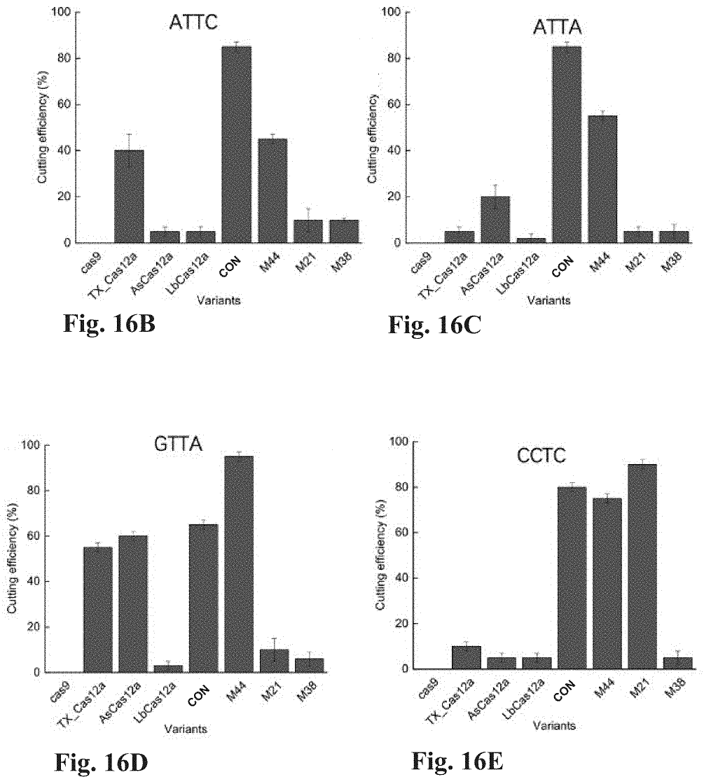

FIGS. 16A-16E illustrate in certain experiments, (A) a schematic illustration of an exemplary plasmid construct and an enlarged view of a specified region of an exemplary KanR region and in (B)-(E), cutting efficiency is assessed by individual verification of unknown PAMs using different nucleases including chimera Cas12a-like nucleases (B)ATTC (C) ATTA (D) GTTA and (E) CCTC.

### Materials and Methods

In certain methods chimeric constructs were created by strategies disclosed herein using at least two Cas12a nuclease molecules to create a chimeric Cas12a nuclease. For example, certain chimeric constructs created by methods disclosed herein are referred to as CU_CH1, CU_CH2, CU_CH3, CU_CH4, CU_CH5, CU_CH6, CU_CH7, CU_CH8, and CU_CH9, where each construct was generated using cross-over technologies to create a chimera derived from peptide fragments of two or more different Cas12a nucleases. In certain methods, off-targeting efficiency rates were evaluated for each chimera Cas12a compared to a control Cas12a to demonstrate improved off-targeting rates. Constructs disclosed and claimed herein include, but are not limited to, CU_CH1: 1 to 927 bp from PC CAS12A, 928 to 3876 bp from a positive control derived from a Cas12a of Eubacterium rectale; CU_CH2: 1 to 912 bp from SC_CAS12A, 913 to 3861 bp from a positive control derived from a Cas12a of Eubacterium rectale; CU_CH3: 1 to 861 bp from FB_CAS12A, 862 to 3810 bp from a positive control derived from a Cas12a of Eubacterium rectal; CU_CH4:1 to 504 bp from TX_CAS12A, 505 to 3819 bp from a positive control derived from a Cas12a of Eubacterium rectale; CU_CH5: 1 to 900 bp from TX_CAS12A with mutation G218A, 901 to 3849 bp from a positive control derived from a Cas12a of Eubacterium rectale; CU_CH6: 1 to 900 bp from TX_CAS12A, 901 to 3174 bp from a positive control derived from a Cas12a of Eubacterium rectale; CU_CH7: 1 to 840 bp from, 841 to 3789 bp from a positive control derived from a Cas12a of Eubacterium rectale; CU_CH8 (M43): 1 to 846 bp from a Cas12a, 847 to 3795 bp from a positive control derived from a Cas12a of Eubacterium rectale; and CU_CH9: 1 to 900 bp from TX_CAS12A, 901 to 3849 bp from a positive control derived from a Cas12a of Eubacterium rectale and combinations thereof.

### Nuclease-Mediated Cell Killing Assay

A two plasmid system was constructed for genome editing, which expresses a Cas12a like protein and a single crRNA (with J23119 promoter) targeting the galK or lacZ gene. For each experiment, equal amounts were transformed of non-targeting and on-targeting (e.g. galK1) gRNA plasmids. The cutting efficiency was calculated as following:

$$Cutting efficiency = ( 1 - \frac{a}{b} ) \times 100 \%$$

The same amount of culture was plated in two LB agar plates with chloramphenicol and carbenicillin. ‘a’ denotes the number of colonies that can grow on the plate with on-targeting gRNA plasmid, and ‘b’ is the number of colonies that can grow on the plate with non-targeting gRNA plasmid.

### Cas12a PAM Screen

PAM plasmid libraries were constructed using synthesized oligonucleotides (IDT) containing the designed NNNN PAM library. The dsDNA product was assembled into a linearized plasmid (containing kanR gene) using Gibson cloning (New England Biolabs). The PAM library was transformed into MG1655 with the plasmid expressing chimeric Cas12a like proteins using the electroporation method. We then transformed two equivalent gRNA plasmids individually into the E. coli MG1655. One gRNA design is targeted on the library sites, and another gRNA plasmid is non-targeting control. We collected the cells grown on kanamycin media using different gRNA plasmids, and amplified the region of the PAM library from the reported plasmid for the high throughput sequencing. The enrichment score of PAM and accompanying sequence logo for one of two library replicates were demonstrated in PAM screening revealed the PAM specificity were different between different chimeric Cas12a like proteins. The prepared cDNA libraries were sequenced on a MiSeq with a single-end 300 cycle kit (Illumina). Indels were mapped using a Python implementation of the Geneious 6.0.3 Read Mapper.

$${E}_{i} = \frac{log ( {Y}_{i} )}{log ( {X}_{i} )}$$

Eᵢ denotes the enrichment score. Xᵢ is the frequency of PAM i using on-targeting gRNA plasmid in the deep sequencing measurements. Yᵢ is the frequency of PAM i using non-targeting gRNA plasmid in the deep sequencing measurements.

### Yeast Transformation

High-efficiency yeast transformation was conducted using the LiAc/SS carrier DNA/PEG method.

### PEI Transfection

HEK293T were cultured in 6-well dish with 60% confluency. After cells attached on the surface of the dish, for each well, two 1.5 mL centrifuge tubes were loaded with 250 μL serum-free and phenol red-free DMEM. One of the tubes was loaded with 3 μL of polyehtyleimine (PEI, concentration: lmg/mL), and the other one tube was loaded with 1 μg of plasmid. After addition, tubes were mixed and placed for 4 min. After placing, tubes loaded with PEI were mixed to tubes with specific plasmid drop-wisely. Tubes were placed for 20 minutes after mixing and mixtures were added into wells drop-wisely.

### Fluorescence-Activated Cell Sorting (FACS)

HEK293T was incubated with 1 mL (0.5%) trypsin at 37° C. for 5 minutes followed by pelleting and resuspension in DMEM with 5% fetal bovine serum (FBS). Resuspended cells were filtered with CellTrics® 50 μm filter to discard debris. Cell sorting was performed using BD FACSAriaTM Fusion equipped with OBIS 488 nm laser (SN: 177745) at 98.3 mW of power. Forward scatter area (FSC-A), side scatter area (SSC-A) and side scatter width (SSC-W) were collected through a filter. The GFP signal was collected in the 488 nm channel through a 530/30-A band pass filter. The first gate was drawn in the SSC-A/FSC-A plot to include cells with universal size, and the second gate was drawn in the SSC-A/SSC-W plot to include single cells. The third gate was drawn in the FSC-A/488 B 530/30-A channel to sort cells with GFP signal.

### T7E1 Assay

Genomic DNA was extracted using the QuickExtract DNA Extraction Solution (Epicenter) following the manufacturer's protocol. The genomic region flanking the CRISPR target site for each gene was PCR amplified, and products were purified using QiaQuick Spin Column (QIAGEN) following the manufacturer's protocol. 200-500 ng total of the purified PCR products were mixed with 1 μl 10×Taq DNA Polymerase PCR buffer (Enzymatics) and ultrapure water to a final volume of 10 μl and were subjected to a re-annealing process to enable heteroduplex formation: 95° C. for 10 min, 95° C. to 85° C. ramping at −2° C./s, 85° C. to 25° C. at −0.25° C./s, and 25° C. hold for 1 min. After re-annealing, products were treated with SURVEYOR nuclease and SURVEYOR enhancer S (Integrated DNA Technologies) following the manufacturer's recommended protocol and analyzed on 4%-20% Novex TBE polyacrylamide gels (Life Technologies). Gels were stained with SYBR Gold DNA stain (Life Technologies) for 10 min and imaged with a Gel Doc gel imaging system (Bio-rad). Quantification was based on relative band intensities. Indel percentage was determined by the formula, 100×(1−sqrt(1−(b+c)/(a+b+c))), where a is the integrated intensity of the undigested PCR product, and b and c are the integrated intensities of each cleavage product.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. An engineered chimeric nucleic acid guided nuclease construct comprising, a construct represented by a nucleic acid sequence having 80% or more homology to a nucleic acid sequence represented by at least one of SEQ ID NO:1 to SEQ ID NO: 9.

2-4. (canceled)

5. The engineered chimeric nucleic acid guided nuclease construct according to claim 1, wherein the construct contains one or more mutations to increase genome editing efficiency.

6. The engineered chimeric nucleic acid guided nuclease construct according to claim 5, wherein the one or more mutations comprise one or more single nucleotide polymorphism(s) (SNP).

7. (canceled)

8. The engineered chimeric nucleic acid guided nuclease construct according to claim 1, wherein the construct has at least one of reduced off-targeting rates for genome editing compared to a control Cas12a-type nucleic acid guided nuclease, increased targeting specificity for genome editing compared to a control Cas12a-type nucleic acid guided nuclease and altered protospacer adjacent motif (PAM) specificity compared to a control Cas12a-type PAM specificity.

9-10. (canceled)

11. The engineered chimeric nucleic acid guided nuclease construct according to claim 1, wherein the construct recognizes a protospacer adjacent motif (PAM) recognized by a control Cas12a-type nuclease having improved off-targeting rates compared to the control Cas12a-type nuclease.

12. (canceled)

13. An engineered chimeric nucleic acid guided nuclease construct comprising, a construct represented by a sequence having 85% or more homology to an amino acid sequence encoded by the polypeptide sequence represented by SEQ ID NO: 28 to SEQ ID NO:36.

14-16. (canceled)

17. The engineered chimeric nucleic acid guided nuclease construct according to claim 13, wherein the construct contains one or more mutations to increase genome editing efficiency.

18. The engineered chimeric nucleic acid guided nuclease construct according to claim 17, wherein the one or more mutations comprise one or more single nucleotide polymorphism(s) (SNP).

19. The engineered chimeric nucleic acid guided nuclease construct according to claim 13, wherein the construct has at least one of increased editing efficiency, reduced off-targeting rates for genome editing, increased targeting specificity for genome editing compared to a control Cas12a-type nucleic acid guided nuclease.

20-21. (canceled)

22. The engineered chimeric nucleic acid guided nuclease construct according to claim 13, wherein the construct has an altered protospacer adjacent motif (PAM) specificity compared to a control Cas12a-type PAM specificity.

23. (canceled)

24. A method for modifying expression of at least one gene product comprising: introducing into a prokaryotic or eukaryotic cell containing and expressing a DNA molecule having a target sequence and encoding the gene product, an engineered chimeric nucleic acid guided nuclease system comprising one or more vectors comprising:
a) a first regulatory element operable in a prokaryotic or eukaryotic cell operably linked to at least one nucleotide sequence encoding a guide RNA system that hybridizes with the target sequence, and
b) a second regulatory element operable in a prokaryotic or eukaryotic cell operably linked to an engineered chimeric nucleic acid guided nuclease construct represented by an engineered chimeric nucleic acid guided nuclease according to claim 1 encoding an engineered chimeric nucleic acid guided nuclease construct polypeptide,
wherein the elements of (a) and (b) are located on same or different vectors of the system, whereby the guide RNA targets the target sequence and the engineered chimeric nucleic acid guided nuclease protein nicks the DNA molecule, whereby expression of the at least one gene product is altered.

25. The method according to claim 24, wherein the method further comprises an insertion of one or more nucleic acids into the target sequence.

26. (canceled)

27. The method according to claim 24, wherein the engineered chimeric nucleic acid guided nuclease protein is codon optimized for expression in the eukaryotic cell.

28. (canceled)

29. The method according to claim 24, wherein cell is a prokaryotic cell.

30-33. (canceled)

34. The method according to claim 24, wherein the one or more vectors are viral vectors.

35-36. (canceled)

37. A vector comprising: an engineered chimeric nucleic acid guided nuclease construct according to claim 1.

38-39. (canceled)

40. A polypeptide encoded by any one of the engineered chimeric nucleic acid guided nuclease constructs according to claim 1.

41. A kit comprising:
one or more containers; and
one or more engineered chimeric nucleic acid guided nuclease construct according to claim 1.

42. The kit according to claim 41, further comprising at a composition comprising a guide RNA.

43. A pharmaceutical composition comprising one or more engineered chimeric nucleic acid guided nuclease construct(s) according to claim 1; and a pharmaceutically acceptable excipient or buffer.
